(19) United States
(12) Patent Application Publication
(10) Pub. No.: US 2007/0087406 A1
(43) Pub. Date: Apr. 19, 2007

(54) Isoforms of receptor for advanced glycation end products (RAGE) and methods of identifying and using same

(76) Inventors: Pei Jin, Palo Alto, CA (US); H. Michael Shepard, San Francisco, CA (US)

Correspondence Address: FISH & RICHARDSON, PC, P.O. BOX 1022, MINNEAPOLIS, MN, 55440-1022, US

(21) Appl. No.: 11/429,090

(22) Filed: May 4, 2006

Related U.S. Application Data
(60) Provisional application No. 60/678,076, filed on May 4, 2005.
(60) Provisional application No. 60/736,134, filed on Nov. 10, 2005.

(51) Int. Cl. A61K 38/17; C07K 14/705; C07H 21/04; C12P 21/06

(52) U.S. Cl. 435/69.1; 435/320.1; 435/325; 530/350; 536/23.5; 514/12

(57) ABSTRACT

Isoforms of RAGE and pharmaceutical compositions containing RAGE isoforms are provided. Methods for identifying and preparing RAGE isoforms are provided. Also provided are methods of treatment with the RAGE isoforms.

### RELATED APPLICATIONS

Benefit of priority is claimed to U.S. provisional application Ser. No. 60/678,076, to Pei Jin and H. Michael Shepard, filed May 4, 2005, entitled “ISOFORMS OF RECEPTOR FOR ADVANCED GLYCATION END PRODUCTS (RAGE) AND METHODS OF IDENTIFYING AND USING SAME,” and to U.S. provisional application Ser. No. 60/736,134, to Pei Jin, H. Michael Shepard, Cornelia Gorman, and Juan Zhang, filed Nov. 10, 2005, entitled “METHODS FOR PRODUCTION OF RECEPTOR AND LIGAND ISOFORMS.” The subject matter of each of these applications is incorporated by reference in its entirety.

This application is related to International PCT Application No. (Attorney Docket No. 17118-040W01/2821PC), filed May 4, 2006, entitled “ISOFORMS OF RECEPTOR FOR ADVANCED GLYCATION END PRODUCTS (RAGE) AND METHODS OF IDENTIFYING AND USING SAME” to Receptor Biologix, Pei Jin and H. Michael Shepard, which also claims priority to U.S. Provisional Application Ser. No. 60/678,076 and to U.S. Provisional Application Ser. No. 60/736,134.

This application also is related to U.S. application Ser. No. 11/129,740 to Pei Jin and H. Michael Shepard, entitled “CELL SURFACE RECEPTOR ISOFORMS AND METHODS OF IDENTIFYING AND USING SAME,” filed May 13, 2005, and to corresponding published International PCT application No WO 05/113596, published Dec. 1, 2005, which claim benefit to U.S. Provisional Application Ser. No. 60/666,825 to Pei Jin and H. Michael Shepard, filed Mar. 30, 2005; to U.S. Provisional Application Ser. No. 60/571,289, filed May 14, 2004, entitled “CELL SURFACE RECEPTOR ISOFORMS AND METHODS OF IDENTIFYING AND USING SAME,” to Pei Jin; and to U.S. Provisional Application Ser. No. 60/580,990, filed Jun. 18, 2004, entitled “CELL SURFACE RECEPTOR ISOFORMS AND METHODS OF IDENTIFYING AND USING SAME,” to Pei Jin. This application also is related to U.S. application Ser. No. 10/846,113, filed May 14, 2004, and to corresponding published International PCT application No. WO 05/016966, published Feb. 24, 2005.

The subject matter of each of the above-referenced related applications, international applications, provisional applications and published applications is incorporated by reference in its entirety.

### FIELD OF THE INVENTION

Isoforms of RAGE and pharmaceutical compositions containing isoforms of RAGE receptor are provided. Methods for identifying and preparing isoforms of RAGE receptors are provided. Also provided are methods of treatment with RAGE receptor isoforms.

### BACKGROUND

Molecules, including small molecules, proteins, lipids and other biological molecules can be altered during cell metabolism and accumulate in cells and tissues over time. In some cases, accumulations of altered molecules can be causative of pathological conditions and disease. In other cases, a disease or condition can result in altered molecule metabolism and lead to the accumulations of particular molecules in altered form and/or amount.

One example is the accumulation of proteins and lipids as glycalated products. The products, referred to as advanced glycation end products (AGEs), are the result of nonenzymatic glycation and oxidation of proteins and lipids in the presence of aldose sugars. Initial early products are formed as reversible Schiff bases and Amadori products. Molecular rearrangements thereof result in irreversible modifications to form AGEs. AGEs accumulate during the normal aging process in humans and AGE accumulation can be accelerated in particular diseases and conditions.

The accumulation of AGEs impact cell and tissue metabolism and signal transduction through their interactions with cellular binding proteins. One such binding protein is the receptor for advanced glycation end products (RAGE). RAGE interaction with AGEs is implicated in induction of cellular oxidant stress responses, including the RAS-MAP kinase pathway and NF-κB activation.

RAGE also binds to other molecules, including small molecules and proteins. S100A12 (also known as EN-RAGE, p6 and calgranulin C) is a calcium binding protein that can act as a ligand for RAGE. RAGE also can interact with β-sheet fibrilar materials including amyloid β-peptides, Aβ, amylin, serum amyloid A and prion-derived peptides. Amphoterin, a heparin-binding neurite outgrowth promoting protein also is a ligand for RAGE. Each of these ligand interactions can affect signal transduction pathways. Diseases and disorders can involve disregulation of and/or changes in the modulation of signal transduction pathways Binding of these ligands to RAGE leads to cellular activation mediated by receptor-dependent signaling to thereby mediate or participate in a variety of diseases and disorders, such as diabetic complications, amyloidoses, inflammatory/immune disorders and tumors.

A goal of drug development is to restore more normal regulation in the signal transduction pathway by targeting such pathways. Because of the involvement of RAGE receptors in a variety of signal transduction pathways that can affect diseases and pathological conditions, RAGE receptors are targets for therapeutic treatments and intervention. Accordingly, among the objects herein, it is an object to provide such therapeutics, methods for identifying or discovering candidate therapeutics, and use of the therapeutics for treatment of disease and disorders.

### SUMMARY

Provided herein are therapeutics that target RAGE receptors and activities. In particular, provided are RAGE isoforms. The RAGE isoforms can modulate the activity of RAGE by interacting with RAGE as a ligand and/or by interacting with RAGE ligands and/or by other mechanisms. Methods of treating RAGE-related disorders and antigiogenic-related disorders are provided.

Hence, provided herein are Receptor for Advanced Glycation Endproducts (RAGE) isoforms. The RAGE isoforms include isoforms that have a V-type Ig-like domain and are modified to have a deletion and/or insertion of one or more amino acids of the second C-type Ig-like domain and a deletion and/or insertion of one or amino acids of the transmembrane domain. Included are RAGE isoforms that exhibit a reduced or abolished membrane localization, particularly soluble isoforms. The isoforms include those that modulate the activity of a RAGE.

RAGE isoforms provided herein include those that contain a sequence of amino acids that has at least 70%, 75%, 80%, 85%, 90%, 95%, 96%, 97%, 98%, 99% or more sequence identity with a sequence of amino acids set forth in SEQ ID NO 10, or 75% 80%, 85%, 90%, 95%, 96%, 97%, 98%, 99% or more sequence identity with a sequence of amino acids set forth in SEQ ID NO. 11, or 86%, 88% 90%, 95%, 96%, 97%, 98%, 99% or more sequence identity with a sequence of amino acids set forth in SEQ ID NO. 12, or 90%, 91%, 92%, 93%, 94%, 95%, 96%, 97%, 98%, 99% or more sequence identity with a sequence of amino acids set forth in SEQ ID NO. 13, or 93%, 94%, 95%, 96%, 97%, 98%, 99% or more sequence identity with a sequence of amino acids set forth in SEQ ID NO. 14 or the recited sequence identity with an allelic or species variant of the isoform set forth in any of SEQ ID Nos. 10-14. Among the RAGE isoforms provided herein are those that include at least one Ig-like domain of RAGE and have a deletion of all of or part of the transmembrane domain so that they are not membrane localize. RAGE isoforms provided herein also include those that lack a signal sequence compared to RAGE. Also provided are RAGE isoforms that contain a signal sequence.

Provided herein are RAGE isoforms polypeptides that include amino acid residues having the sequences of amino acids set forth in any SEQ ID NOS: 10-14 and an allelic and species variants thereof. Allelic variants include, for example, those that contain a sequence of amino acids with one or more amino acid variation as set forth in SEQ ID NO. 4. Also provided are RAGE isoforms that contain the same number of amino acids as set forth in any of SEQ ID NOS: 10-14.

Provided herein are RAGE isoforms that modulate the function or activity of the RAGE receptor. A modulated activity of the RAGE receptor includes, for example, any selected from among ligand binding, competition with RAGE for ligand binding, ligand endocytosis, regulation of gene expression, signal transduction, interaction with a signal transduction molecule, membrane association and membrane localization.

Among the isoforms of RAGE provided herein are those that contain an intron-encoded sequence of amino acids from the gene encoding the RAGE receptor (referred to as intron fusion proteins). The intron-encoded portion can be at either terminus or internally located in the polypeptide Provided, for example, are RAGE isoform polypeptides that contain at least one domain of the RAGE receptor operatively linked to at least one amino acid encoded by an intron of a gene encoding the RAGE receptor or those in which the intron-encoded portion is a stop codon resulting in a truncation at the exon-intron junction.

Provided herein are RAGE isoforms encoded by a sequence of nucleotides set forth in any of SEQ ID NOS: 5-9 and allelic and species variants thereof. Exemplary of the allelic variants are those encoded by a sequence of nucleotides set forth in SEQ ID NO. 3. Also provided are RAGE isoform that contain amino acids encoded by all or part of an intron, including those in which the intron portion contains only a stop codon such that the nucleic acid molecule encodes an open reading frame that spans an exon intron junction and the open reading frame terminates at the stop codon in the intron. Typically the intron encodes one or more amino acids of the encoded RAGE isoforms described herein. In another embodiment, the stop codon is the first codon of the intron.

Provided herein are pharmaceutical compositions including any of the RAGE isoforms provided herein. The pharmaceutical composition can contain an amount of the isoform effective for modulating an activity of a cell surface receptor. In a particular embodiment, the cell surface receptor is RAGE. The modulated activity of the RAGE receptor, for example, is selected from among ligand binding, competition with RAGE for ligand binding, ligand endocytosis, regulation of gene expression, signal transduction, interaction with a signal transduction molecule, membrane association and membrane localization. The modulated activity of the RAGE receptor can be an inhibition of any activity or an enhancement of an activity. In general it is desired to inhibit the activity of RAGE to thereby inhibit any associated pathways an consequent diseases and disorders. Also provided herein, is a composition where the isoform of the composition complexes with RAGE.

Provided herein are nucleic acid molecules encoding the RAGE isoforms provided herein. Among these are nucleic acid molecules having a sequence of nucleic acids set forth in SEQ ID NOS. 5-9 and allelic and species variants thereof. Also provided herein are plasmids vectors containing the nucleic acid molecules. Vectors include mammalian viral vectors. Vectors can be those that remain episomal or integrates into the chromosome of a cell into which they are introduced. Vectors also include artificial chromosomes and other replicating elements. Also provided are cells, prokaryotic and eukaryotic, containing a vector as described herein. Vectors also include artificial chromosomes and other replicating elements. Also provided are pharmaceutical compositions containing the nucleic acid molecules as well as the plasmids and vectors and/or cells. Such compositions can be used in ex vivo and in vivo methods for delivery of genes and gene products to an organism.

Provided herein are methods of treating a disease or condition by administering any of the pharmaceutical compositions. Diseases treated include any in which RAGE and ligands therefor play a role, such as inflammatory and immune disorders. Exemplary diseases include, but are not limited to, diabetes, diabetes related conditions, cancers, inflammatory diseases, angiogenesis-related conditions, cell proliferation-related conditions, immune disorders, kidney disease, ocular disease, endometriosis, periodontal disease and neurodegenerative disease. Additionally, the disease or condition includes, but is not limited to, rheumatoid arthritis, osteoarthritic arthritis, multiple sclerosis, Alzheimer's disease and other neurodegenerative diseases and diseases of protein aggregation, Creutzfeldt-Jakob disease, Huntington's disease, posterior intraocular inflammation, uveitic disorders, ocular surface inflammatory disorders, macular degeneration, neovascular disease, proliferative vitreoretinopathy, atherosclerosis, type I diabetes, and chronic kidney disease. In a particular embodiment, the disease is an angiogenesis-related disease.

Exemplary of diseases treated are diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease. The RAGE isoforms can be used to inhibit tumor invasion or metastasis of a tumor.

In one embodiment, the diabetes-associated condition includes periodontal disease, autoimmune disease, vascular disease, tubulointerstitial disease, atherosclerosis and vascular disease associated with wound healing. In another embodiment, the cancer disease or condition includes carcinoma, lymphoma, blastoma, sarcoma, leukemia, lymphoid malignancies, squamous cell cancer, lung cancer including small-cell lung cancer, non-small cell lung cancer, adenocarcinoma of the lung and squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial/uterine carcinoma, salivary gland carcinoma, renal cancer, prostate cancer, vulval cancer, thyroid cancer, hepatic carcinoma, anal carcinoma, penile carcinoma, and head and neck cancer.

Provided herein is a conjugates that contain a RAGE isoform. The conjugates include a RAGE isoforms linked directly or via a linker to another molecule, such as a biomolecule or macromolecule, such as serum albumin, a drug, an other receptor isoforms or portion thereof,

Provided herein are chimeric polypeptides (or conjugates) that contain all or at least one domain of a RAGE isoform and all or at least one domain of a different RAGE isoform or of another cell surface receptor isoform. In one embodiment, the cell surface receptor isoform is an intron fusion protein. In another embodiment, the polypeptide contains all or at least one domain of a RAGE isoform and an intron-encoded portion of a cell surface receptor isoform. Provided herein are polypeptides containing a domain of RAGE linked directly or indirectly to serum albumin. In one embodiment, the RAGE isoform is an intron fusion protein and the domain is the intron portion. Also provided are chimeric conjugates that contain two or more isoforms described herein, including the RAGE isoforms and other receptor isoforms. The components of the chimeras and conjugates can be linked via peptide bonds, other covalent linkages, such as hydrogen bonding, van der waals forces and other such interactions, such as those responsible for antigen/antibody interactions, ligand bonding and other such interactions. Linkage can be direct or indirect via one or more linkers.

Provided herein is a combination that includes one or more of the RAGE isoforms as described herein and one or more other cell surface receptor and/or a therapeutic drug. In one embodiment, the isoforms and/or drugs in the combination are in separate compositions or in a single composition.

Provided herein are methods of treatment that include administering a chimeric polypeptide that contains all or at least one domain of a RAGE isoform and all or at least one domain of a different RAGE isoform or of another cell surface receptor isoform. In one embodiment, each component is administered separately, simultaneously, intermittently, in a single composition or combinations thereof.

Provided herein are pharmaceutical compostions that contain a nucleic acid molecule including a nucleic acid encoding a RAGE isoform provided herein.

Provided herein are pharmaceutical compositions that contain a nucleic acid molecule including a nucleic acid molecule encoding a RAGE isoform. Also provided area pharmaceutical compositions that contain nucleic acid molecules encoding RAGE isoforms that contains an intron and an exon (an intron fusion protein).

Also provided are therapeutic methods that include administering a pharmaceutical composition provided herein that contains nucleic acid encoding a RAGE isoform or portion thereof. The composition can be introduced into a cell that has been removed from a host animal and reintroduced into the same animal or an animal compatible or treated to be compatible with the cells. In another embodiment, the composition is introduced into an animal. In a particular embodiment, the animal is a human.

### BRIEF DESCRIPTION OF THE FIGURES

FIG. 1 depicts angiogenic and endothelial cell maintenance pathways. The figure depicts the targets for AGEs, which act via interaction with RAGE. Hence, target points for modulation of these pathways by RAGE isoforms are indicated.

### DETAILED DESCRIPTION

Outline

A. DEFINITIONS

B. Receptor for Advanced Glycation Endproducts
1. RAGE

C. RAGE Receptor Isoforms
1. Identification and production of RAGE isoforms
   a. Alternative Splicing and Generation of RAGE Isoforms
      i. Isoforms generated by intron modification
      ii. Isoforms generated by exon modifications
   b. In silico generated RAGE isoforms
2. RAGE Isoform Polypeptide Structure
3. RAGE isoform biological activities
   a. Negatively acting and inhibitory isoforms

D. Methods for identifying and generating RAGE isoforms

E. Exemplary RAGE isoforms
1. Allelic Variants of Isoforms

F. Methods of Producing Nucleic Acid Encoding RAGE Isoforms and Methods of Producing

RAGE Polypeptides
1. Synthetic genes and polypeptides
2. Methods of cloning and isolating RAGE isoforms
3. Expression Systems
   a. Prokaryotic expression
   b. Yeast
   c. Insect Cells
   d. Mammalian cells
   e. Plants

G. Isoform Conjugates
1. Isoform Fusions
   a. RAGE Isoform Fusions for Improved Production of RAGE Isoform Polypeptides
      i. Tissue Plasminogen Activator
      ii. tPA-RAGE Isoform Fusions
   b. Chimeric and synthetic RAGE isoform polypeptides including homo- and heteromultimeric polypeptides
   c. Methods of Generating and Cloning RAGE Fusions
2. Targeting Agent/Targeting Agent Conjugates
3. Peptidomimetic isoforms

H. Assays to assess or monitor RAGE isoform activities
1. Ligand Binding Assays and RAGE binding assays
2. Complexation
3. Gene Expression Assays
4. Cell Proliferation Assays
5. ERK Phosphorylation Assays
6. Cell Migration Assay
7. Neurite Outgrowth
8. Animal Models
   a. Diabetic vasculopathy
   b. Diabetic atherosclerosis
   c. Diabetic inflammatory bone loss
   d. Autoimmune diabetes

I. Preparation, Formulation and Administration of RAGE isoforms and RAGE isoform compositions

J. In Vivo Expression of RAGE isoforms and Gene Therapy
1. Delivery of nucleic acids
   a. Vectors-episomal and integrating
   b. Artificial chromosomes and other non-viral vector delivery methods
   c. Liposomes and other encapsulated forms and administration of cells containing the nucleic acids
2. In Vitro and Ex Vivo delivery
3. Systemic, local and topical delivery

K. RAGE and Angiogenesis
1. Angiogenesis and disease
2. The angiogenic process
3. Cell Surface receptors in Angiogenesis
4. Cell Surface receptors in tumors
5. RAGE and RAGE ligands in Angiogenesis
6. RAGE isoforms and angiogenesis

L. Exemplary Treatments with RAGE isoforms
1. Age-related macular degeneration
2. Diabetes related diseases
   a. Vascular Disease
   b. Periodontal Disease
   c. Endometriosis
3. Autoimmune Disease
4. Neurodegenerative Disease
5. Cardiovascular Disease
6. Kidney Disease
7. Arthritis
8. Cancer
9. Combination Therapies
10. Evaluation of RAGE isoform activities

M. EXAMPLES

A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a cell surface receptor is a protein that is expressed on the surface of a cell and typically includes a transmembrane domain or other moiety that anchors it to the surface of a cell. As a receptor it binds to ligands that mediate or participate in an activity of the cell surface receptor, such as signal transduction or ligand internalization. Cell surface receptors include, but are not limited to, single transmembrane receptors and G-protein coupled receptors. Receptor tyrosine kinases, such as growth factor receptors, also are among such cell surface receptors.

As used herein, Advanced glycation end products (AGE) are adducts formed by the non-enzymatic glycation or oxidation of macromolecules. AGE forms, for example, during aging and formation is accelerated under pathophysiologic states such as diabetes, Alzheimer's disease, renal failure, immune/inflammatory disorders and other diseases and disorders.

As used herein, RAGE refers to Receptor for Advanced Glycation Endoproducts (RAGE) that is named for its ability to bind AGE. RAGE is a multiligand receptor belonging to the immunoglobulin (Ig) superfamily. RAGE binds to other products, including amyloid β-peptide, S100/calgranulin family proteins, high mobility group B1 (HMGB1, also know as amphoterin) and leukocyte integrins. As an example, a human RAGE gene encodes a 404 amino acid residue (aa) type I transmembrane glycoprotein with a 22 aa signal peptide, a 319 aa extracellular domain containing an Ig-like V-type domain and two Ig-like C-type domains, a 21 aa transmembrane domain and a 40 aa cytoplasmic domain (see SEQ ID No: 2). The V-type domain and the cytoplasmic domain are important for ligand binding and for intracellular signaling, respectively. The RAGE gene is composed of 11 exons interrupted by 10 introns. An exemplary genomic sequence of RAGE is set forth as SEQ ID NO:325. Alternative splice variants of RAGE exist. For example, two alternative splice variants, lacking the V-type domain or the cytoplasmic tail, are known. Sequences of exemplary RAGE isoforms, including alternative splice variants of RAGE, are set forth in SEQ ID NOS: 292-305. RAGE includes allelic variants of RAGE, such as any one of the allelic variants of a RAGE polypeptide or nucleic acid, such as set forth in SEQ ID NOS: 3 and 4, respectively. RAGE is also found in different species, and thus includes species variants.

RAGE is highly expressed in the embryonic central nervous system. In adult tissues, RAGE is expressed at low levels in multiple tissues including endothelial and smooth muscle cells, mononuclear phagocytes, pericytes, microglia, neurons, cardiac myocytes and hepatocytes. The expression of RAGE is upregulated upon ligand interaction. Depending on the cellular context and interacting ligand, RAGE activation can trigger differential signaling pathways that affect divergent pathways of gene expression. RAGE activation modulates varied essential cellular responses (including inflammation, immunity, proliferation, cellular adhesion and migration) that contribute to cellular dysfunction associated with chronic diseases such as diabetes, cancer, amyloidoses and immune or inflammatory disorders and other proliferative and degenerative diseases, including neurodegenerative diseases and endometriosis. RAGE receptors are implicated in induction of cellular oxidant stress responses, including via the RAS-MAP kinase pathway and NF-κB activation.

As used herein, a domain refers to a portion (a sequence of three or more, generally 5 or 7 or more amino acids) of a polypeptide that is a structurally and/or functionally distinguishable or definable. For example, a domain includes those that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domain. For example, a domain can be identified, defined or distinguished by homology of the sequence therein to related family members, such as homology and motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, RAGE typically includes three immunoglobulin-like domains, a membrane-spanning (transmembrane) domain and an intracellular domain. Those of skill in the art are familiar with such domains and can identify them by virtue of structural and/or functional homology with other such domains.

As used herein an Ig-like domain is a domain recognized as such by those of skill in the art and is a domain containing folds of beta strands forming a compact folded structure of two beta sheets stabilized by hydrophobic interactions and sandwiched together by an intra-chain disulfide bond. In one example, an Ig-like C-type domain contains seven beta strands arranged as four-strand plus three-strand so that four beta strands form one beta sheet and three beta strands form the second beta sheet. For example, RAGE contains two Ig-like C-type domain: the first Ig-like C-type domain corresponds to amino acids 124-221 of a RAGE polypeptide having an amino acid sequence set forth in SEQ ID NO:2, and the second Ig-like C-type domain corresponds to amino acids 227-317 of a RAGE polypeptide having an amino acid sequence set forth in SEQ ID NO:2. In another example, an Ig-like V-type domain contains nine beta strands arranged as four beta strands plus five beta strands (Janeway C. A. et al. (eds): Immunobiology—the immune system in health and disease, 5th edn. New York, Garland Publishing, 2001). For example, RAGE contains one V-type Ig-like domain corresponding to amino acids 23-116 of a RAGE polypeptide having a sequence of amino acids set forth in SEQ ID NO:2.

As used herein, an extracellular domain is the portion of the cell surface receptor that occurs on the surface of the receptor and include the ligand binding site(s). For example, the extracellular domain of a RAGE polypeptide corresponds to amino acids 1-342 of a RAGE polypeptide having a sequence of amino acids set forth in SEQ ID NO:2.

As used herein, a transmembrane domain spans the plasma membrane anchoring the receptor and generally includes hydrophobic residues. For example, a transmembrane domain corresponds to amino acids 342-363 of a RAGE polypeptide having a sequence of amino acids set forth in SEQ ID NO:2.

As used herein, a cytoplasmic domain is a domain that participates in signal transduction. For example, a cytoplasmic domain corresponds to amino acids 364-404 of a RAGE polypeptide having a sequence of amino acids set forth in SEQ ID NO:2.

As used herein, an isoform of RAGE (also referred to herein as a RAGE isoform), refers to a receptor that has an altered polypeptide structure compared to a full-length wildtype (predominant) form of the corresponding RAGE, such as for example, due to differences in the nucleic acid sequence and encoded polypeptide of the isoform compared to the corresponding protein. Generally a RAGE isoform provided herein lacks a domain or portion thereof (or includes insertions or both) sufficient to alter an activity, such as an enzymatic activity, or the structure compared to that of the cognate full-length receptor.

The RAGE isoforms generally lack all or a sufficient portion of the transmembrane domain of a RAGE (and also the cytoplasmic domain) so that the RAGE isoform is not membrane-anchored. In addition, the isoform lacks one or more other domains or portion thereof. Included are isoforms that contain insertions that result in an alteration of an activity of the receptor or that add an activity. In addition, among the RAGE isoforms provided herein are those that are intron-fusion proteins in that they include at least one, typically 2 or more amino acid residues, typically, although not necessarily, at the C-terminal end of the protein, that are encoded by an intron in the gene encoding the corresponding receptor. In some instances, the encoded amino acid can be a stop codon. By virtue of the differences in structure, one or more functions (an activity) also can be altered, eliminated, and/or added. For example, the cytoplasmic domain of RAGE is required for NF-κB-dependent transcription. Elimination thereof, eliminates this activity in a RAGE isoform.

Generally, when an activity is altered in an isoform, it is altered by at least 0.1, 0.5, 1, 2, 3, 4, 5, or 10 fold compared to a wildtype and/or predominant form of the receptor. Typically, an activity is altered 2, 5, 10, 20, 50, 100 or 1000 fold or more. Alteration of activity includes an enhancement or a reduction of activity. In one embodiment, an alteration of an activity is a reduction in an activity; the reduction can be at least 0.1 0.5 1, 2, 3, 4, 5, or 10 fold compared to a wildtype and/or predominant form of the receptor. Typically, an activity is reduced 5, 10, 20, 50, 100 or 1000 fold or more.

An isoform can include a receptor that is shortened or lengthened (with respect to the total length of amino acid sequence compared to a predominant and/or wildtype form of the receptor) or otherwise altered, including a deletion, insertion, amino acid replacement and/or combinations thereof compared to the amino acid sequence of a predominant and/or wildtype form of the receptor. Additions can include an additional domain, such as that encoded by an intron or a portion thereof in the gene encoded in the wildtype. The portion can be 1, 2, 3, 5, 10, 15, 20, 25, 30, 35, 40, 50, 60 or more amino acids. Generally the isoforms provided herein lack all or a sufficient portion of the transmembrane domain to preclude membrane anchoring. Also, the isoforms generally lack another domain and/or include an intron-encoded region.

Thus, provided are RAGE isoforms that typically lack all or part of the transmembrane domain and at least one other domain and/or include insertions, including all or portions of intron-encoded regions. The isoforms also generally are capable of modulating the activity of a RAGE.

The RAGE isoforms provided herein are from any species, including mammals, such as primates, particularly humans, and domesticated animals, including dogs, cats, and others, such as rodents and avian species. For purposes herein, a human RAGE isoform is an isoform that has a cognate human receptor that is encoded by a gene from a human tissue or human cell source.

A RAGE isoform can be produced by any method known in the art including isolation of isoforms expressed in cells, tissues and organisms, and by recombinant methods and by use of in silico and synthetic methods. Isoforms of cell surface receptors, including isoforms of RAGE, can be encoded by alternatively spliced RNAs transcribed from a RAGE gene. Such isoforms include exon deletion, exon extension, exon truncation and intron retention alternatively spliced RNAs.

As used herein, reference herein to modulating the activity of a RAGE means that a RAGE isoform interacts in some manner with the RAGE and an activity of the RAGE, such as ligand binding or other signal-transduction-related activity is altered.

As used herein, an exon refers to a sequence of nucleotides that is transcribed into RNA and is represented in a mature form of RNA, such as mRNA (messenger RNA), after splicing and other RNA processing. An mRNA contains one or more exons operatively linked. Exons can encode polypeptides or a portion of a polypeptide. Exons also can contain non-translated sequences for example, translational regulatory sequences. Exon sequences often are conserved and exhibit homology among gene family members.

As used herein, an intron refers to a sequence of nucleotides that is transcribed into RNA and is then typically removed from the RNA by splicing to create a mature form of an RNA, for example, an mRNA. Typically, introns are not incorporated into mature RNAs, nor are introns sequences or a portion thereof typically translated and incorporated into a polypeptide. Splice signal sequences, such as splice donors and acceptors, are used by the splicing machinery of a cell to remove introns from RNA. It is noteworthy that an intron in one splice variant can be an exon (i.e., present in the spliced transcript) in another variant. Hence, spliced mRNA encoding an intron fusion protein can include an exon(s) and introns.

As used herein, splicing refers to a process of RNA maturation where introns in the mRNA are removed and exons are operatively linked to create a messenger RNA (mRNA).

As used herein, alternative splicing refers to the process of producing multiple mRNAs from a gene. Alternate splicing can include operatively linking less than all the exons of a gene, and/or operatively linking one or more alternate exons that are not present in all transcripts derived from a gene.

As used herein, a gene refers to a sequence of nucleotides transcribed into RNA (introns and exons), including nucleotide sequence that encodes at least one polypeptide. A gene includes sequences of nucleotides that regulate transcription and processing of RNA. A gene also includes regulatory sequences of nucleotides such as promoters and enhancers, and translation regulation sequences. Genes also can include exons and introns.

As used herein, a cognate gene with reference to an encoded polypeptide provided herein refers to the gene sequence that encodes a predominant polypeptide and is the same gene as the particular isoform. For purposes herein a cognate gene can include a natural gene or a gene that is synthesized such as by using recombinant DNA techniques. Generally, the cognate gene also is a predominant form in a particular cell or tissue.

As used herein, a cognate polypeptide or receptor with reference to the isoforms provided herein refers to the receptor that is encoded by the same gene as the particular isoform. Generally, the cognate receptor also is a predominant form in a particular cell or tissue. For example, herstatin is encoded by a splice variant of the pre-mRNA which encodes p185-HER2 (erbb2 receptor). Thus, p185-HER2 is the cognate receptor for herstatin. For purposes herein, the cognate receptor is a RAGE receptor, generally the full-length or predominant form of RAGE.

As used herein, a wildtype form, for example, a wildtype form of a polypeptide, refers to a polypeptide that is encoded by a gene. Typically a wildtype form refers to a gene (or RNA or protein derived therefrom) without mutations or other modifications that alter function or structure; wildtype forms include allelic variation among and between species. For purposes herein, the wildtype form of RAGE is set forth in SEQ ID NO:2, and encoded by a sequence of nucleotides set forth in SEQ ID NO:1. The wildtype RAGE includes allelic or species variation, such as for example any one or more of the allelic variants set forth in SEQ ID NO: 3 and 4.

As used herein, a predominant form, for example, a predominant form of a polypeptide, refers to a polypeptide that is the major polypeptide produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form.”

As used herein, a splice site refers to one or more nucleotides within the gene that participate in the removal of an intron and/or the joining of an exon. Splice sites include splice acceptor sites and splice donor sites.

As used herein, exon deletion refers to an event of alternative RNA splicing that produces a nucleic acid molecule that lacks at least one exon compared to an RNA encoding a wildtype or predominant form of a polypeptide.

As used herein, exon insertion, also referred as exon retention, refers to an event of alternative RNA splicing that produces a nucleic acid molecule that contains at least one exon not typically present in an RNA encoding a wildtype or predominant form of a polypeptide.

As used herein, exon extension refers to an event of alternative RNA splicing that produces a nucleic acid molecule that contains at least one exon that is greater in length (number of nucleotides contained in the exon) than the corresponding exon in an RNA encoding a wildtype or predominant form of a polypeptide. In some cases, as described further herein, an mRNA produced by exon extension encodes an intron fusion protein.

As used herein, exon truncation refers to an event of alternative RNA splicing that produces a nucleic acid molecule that contains a truncation or shortening of one or more exons such that the one or more exons are shorter in length (number of nucleotides) compared to a corresponding exon in an RNA encoding a wildtype or predominant form of a polypeptide.

As used herein intron retention refers to an event of alternative RNA splicing that produces a nucleic acid molecule that contains an intron or a portion thereof operatively linked to one or more exons. In some cases, as described further herein, an mRNA produced by intron retention encodes an intron fusion protein.

As used herein, an intron fusion protein refers to an isoform encoded by a nucleic acid molecule that includes at least one codon (including stop codons) from one or more introns resulting either in truncation of a polypeptide isoform at the end of an exon operatively linked to the intron-encoded portion, or in an addition of one, 2, 3, 4, 5, 8, 10, 15, 20, 25, 30, 35, 40, 45, 50, 55, 60, 65, 70, 75 and more amino acids encoded by an intron. Generally, an intron fusion protein is encoded by nucleic acids that contain one or more codons (with reference to the predominant or wildtype form of a protein), including stop codons, operatively linked to exon codons. The intron portion can be a stop codon, resulting in an intron fusion protein that ends at the exon intron junctions. The activity of an intron fusion protein typically is different from the predominant form of a polypeptide, generally by virtue of truncations, deletions and/or insertion due to the presence of the intron(s) encoded amino acid residues. Typically, such truncations or deletions results in an isoform that lacks one or more domain(s) or portion of one or more domain(s) resulting in an alteration of an activity of a receptor. The activity can be altered by the intron fusion protein directly, such as by interaction with the receptor, or indirectly by interacting with a receptor ligand or co-factor or other modulator of receptor activity. Intron fusion proteins can occur in cells and tissues and can be encoded by an alternatively spliced RNA. In addition, intron fusion proteins can be encoded by RNA molecules identified in silico by identifying potential splice sites and then produced by recombinant methods or they can be prepared synthetically. Typically, an intron fusion protein is shortened compared to a RAGE by the presence of one or more stop codons in an intron fusion protein-encoding RNA that are not present in the corresponding sequence of an RNA encoding a wildtype or predominant form of a corresponding RAGE polypeptide. Addition of amino acids and/or a stop codons can result in an intron fusion protein that differs in size and sequence from a wildtype or predominant form of a polypeptide.

As used herein, a polypeptide lacking all or a portion of a domain refers a polypeptide that has a deletion of one or more amino acids or all of the amino acids of a domain compared to a cognate polypeptide. Amino acids deleted in a polypeptide lacking all or part of a domain need not be contiguous amino acids within the domain of the cognate polypeptide. Polypeptides that lack all or a part of a domain can include the loss or reduction of an activity of the polypeptide compared to the biological activity of a cognate polypeptide, or loss of a structure in the polypeptide.

For example, if a cognate receptor has a transmembrane domain between amino acids 400-420, then a receptor isoform polypeptide lacking all or a part of the transmembrane domain can have a deletion of 1, 2, 3, 4, 5, 6, 7, 8, 9, 10, 11, 12, 13, 14, 15, 16, 17, 18, 19, 20 or more amino acids between amino acids corresponding to amino acid positions 400-420 of the cognate receptor. Generally the isoforms provided herein lack all or a sufficient portion of the transmembrane domain to be secreted such that they are not anchored in the membrane.

As used herein, a polypeptide comprising a domain refers to a polypeptide that contains a complete domain with reference to the corresponding domain of a cognate receptor. A complete domain is determined with reference to the definition of that particular domain within a cognate polypeptide. For example, a receptor isoform comprising a domain refers to an isoform that contains a domain corresponding to the complete domain as found in the cognate receptor. If a cognate receptor, for example, contains a transmembrane domain of 21 amino acids between amino acid positions 400-420, then a receptor isoform that comprises such transmembrane domain, contains a 21 amino acid domain that has substantial identity with the 21 amino acid domain of the cognate receptor. Substantial identity refers to a domain that can contain allelic variation and conservative substitutions as compared to the domain of the cognate receptor. Domains that are substantially identical do not have deletions, non-conservative substitutions or insertions of amino acids compared to the domain of the cognate receptor.

As used herein, an allelic variant or allelic variation references to a polypeptide encoded by a gene that differs from a reference form of a gene (i.e. is encoded by an allele). Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide.

As used herein, species variants refer to variants of the same polypeptide between and among species. Generally, interspecies variants have at least about 60%, 70%, 80%, 85%, 90%, or 95% identity or greater with a wildtype and/or predominant form from another species, including 96%, 97%, 98%, 99% or greater identity with a wildtype and/or predominant form of a polypeptide.

As used herein, modification in reference to modification of a sequence of amino acids of a polypeptide or a sequence of nucleotides in a nucleic acid molecule and includes deletions, insertions, and replacements of amino acids and nucleotides, respectively.

As used herein, an open reading frame refers to a sequence of nucleotides or ribonucleotides in a nucleic acid molecule that encodes a functional polypeptide or a portion thereof, typically at least about fifty amino acids. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, truncation or shortening with reference to the shortening of a nucleic acid molecule or protein, refers to a sequence of nucleotides or ribonucleotides in a nucleic acid molecule or a sequence of amino acid residues in a polypeptide that is less than full-length compared to a wildtype or predominant form of the protein or nucleic acid molecule.

As used herein, a reference gene refers to a gene that can be used to map introns and exons within a gene. A reference gene can be genomic DNA or portion thereof, that can be compared with, for example, an expressed gene sequence, to map introns and exons in the gene. A reference gene also can be a gene encoding a wildtype or predominant form of a polypeptide.

As used herein, a family or related family of proteins or genes refers to a group of proteins or genes, respectively that have homology and/or structural similarity and/or functional similarity with each other.

As used herein, a premature stop codon is a stop codon occurring in the open reading frame of a nucleic acid molecule before the stop codon used to produce or create a full-length form of a protein, such as a wildtype or predominant form of a polypeptide. The occurrence of a premature stop codon can be the result of, for example, alternative splicing and mutation.

As used herein, an expressed gene sequence refers to any sequence of nucleotides transcribed or predicted to be transcribed from a gene. Expressed gene sequences include, but are not limited to, cDNAs, ESTs, and in silico predictions of expressed sequences, for example, based on splice site predictions and in silico generation of spliced sequences.

As used herein, an expressed sequence tag (EST) is a sequence of nucleotides generated from an expressed gene sequence. ESTs are generated by using a population of mRNA to produce cDNA. The cDNA molecules can be produced for example, by priming from the polyA tail present on mRNAs. cDNA molecules also can be produced by random priming using one or more oligonucleotides which prime cDNA synthesis internally in mRNAs. The generated cDNA molecules are sequenced and the sequences are typically stored in a database. An example of an EST database is dbEST found online at ncbi.nlm.nih.gov/dbEST. Each EST sequence is typically assigned a unique identifier and information such as the nucleotide sequence, length, tissue type where expressed, and other associated data is associated with the identifier.

As used herein, a kinase is a protein that is able to phosphorylate a molecule, typically a biomolecule, including macromolecules and small molecules. For example, the molecule can be a small molecule, or a protein. Phosphorylation includes auto-phosphorylation. Some kinases have constitutive kinase activity. Other kinases require activation. For example, many kinases that participate in signal transduction are phosphorylated. Phosphorylation activates their kinase activity on another biomolecule in a pathway. Some kinases are modulated by a change in protein structure and/or interaction with another molecule. For example, complexation of a protein or binding of a molecule to a kinase can activate or inhibit kinase activity.

As used herein, designated refers to the selection of a molecule or portion thereof as a point of reference or comparison. For example, a domain can be selected as a designated domain for the purpose of constructing polypeptides that are modified within the selected domain. In another example, an intron can be selected as a designated intron for the purpose of identifying RNA transcripts that include or exclude the selected intron.

As used herein, modulate and modulation refer to a change of an activity of a molecule, such as a protein. Exemplary activities include, but are not limited to, biological activities, such as signal transduction. Modulation can include an increase in the activity (i.e., up-regulation agonist activity) a decrease in activity (i.e., down-regulation or inhibition) or any other alteration in an activity (such as periodicity, frequency, duration, kinetics. Modulation can be context dependent and typically modulation is compared to a designated state, for example, the wildtype protein, the protein in a constitutive state, or the protein as expressed in a designated cell type or condition.

As used herein, inhibit and inhibition refer to a reduction in an activity relative to the uninhibited activity.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related. A kit is a packaged combination that optionally includes instructions for use of the combination or elements thereof.

As used herein, a pharmaceutical effect refers to an effect observed upon administration of an agent intended for treatment of a disease or disorder or for amelioration of the symptoms thereof.

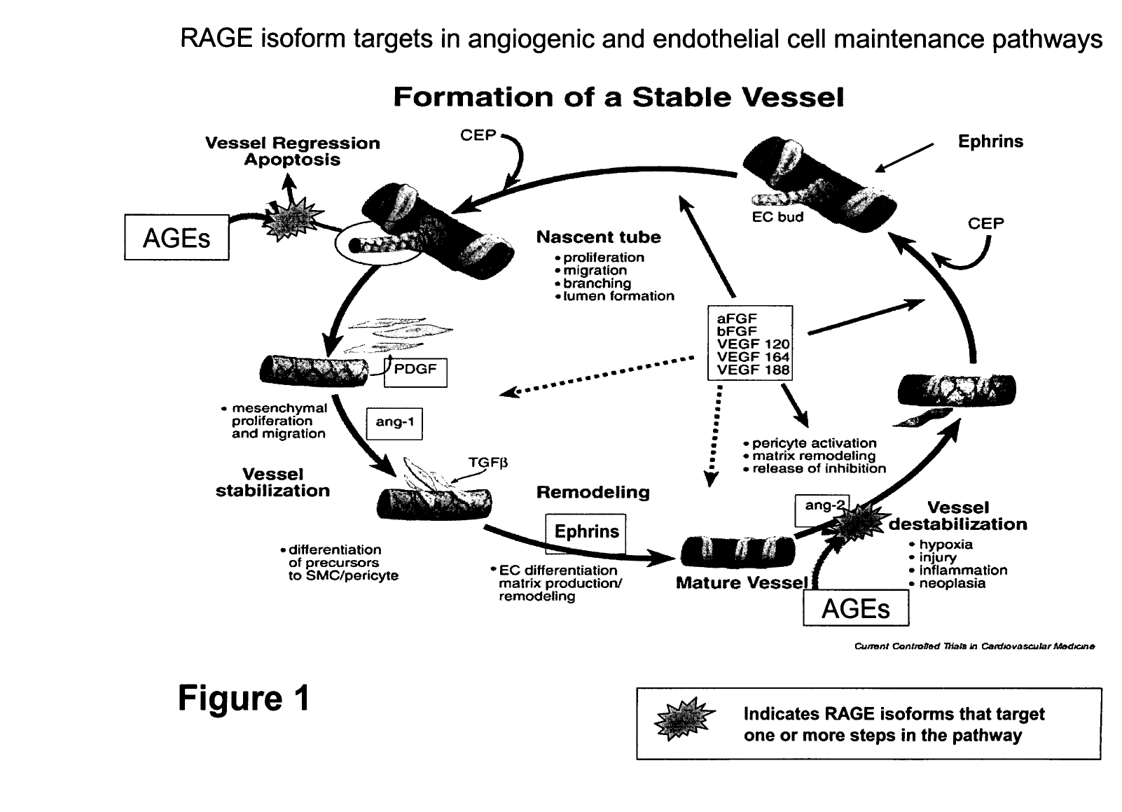

As used herein, angiogenesis refers to the formation of new blood vessels from existing ones; neovascularization refers to the formation of new vessels. Physiologic angiongenesis is tightly regulated and is essential to reproduction and embryonic development. During post natal and adult life, angiogenesis occurs in wound repair and in exercised muscle and is generally restricted to days or weeks. In contrast, pathologic angiogenesis (or aberrant angiogenesis) can be persistent for months or years supporting the growth of solid tumors and leukemias, for example. It provides a conduit for the entry of inflammatory cells into sites of chronic inflammation (e.g., Crohn's disease and chronic cystitis). It is the most common cause of blindness; it destroys cartilage in rheumatoid arthritis and contributes to the growth and hemorrhage of atherosclerotic plaques. It leads to intraperitoneal bleeding in endometriosis. Tumor growth is angiogenesis-dependent. Tumors recruit their own blood supply by releasing factors that stimulate angiogenesis. Such factors include, VEGF, FGF, PDGF, TGF-β, Tek, EPHA2, AGE and others (see, e.g., FIG. 1). AGE-RAGE interactions can elicit angiogenesis through transcriptional activation of the VEGF gene via NF-κB and AP-1 factors. VEGF is overproduced in a large number of human cancers, including breast, lung, colorectal.

As used herein, angiogenic diseases (or angiogenesis-related diseases) are diseases in which the balance of angiogenesis is altered or the timing thereof is altered. Angiogenic diseases include those in which an alteration of angiogenesis, such as undesirable vascularization, occurs. Such diseases include, but are not limited to cell proliferative disorders, including cancers, diabetic retinopathies and other diabetic complications, inflammatory diseases, endometriosis and other diseases in which excessive vascularization is part of the disease process, including those noted above. As noted, the AGE-RAGE interaction elicits angiogenesis through transcriptional activation of the vascular endothelial growth factor (VEGF) gene via NF-κB and AP-1 factors. Hence any disorder involving VEGF interactions with VEGFR is included.

As used herein, RAGE-related diseases are any in which RAGE is implicated in some aspect of the etiology, pathology or development thereof. Diseases, include, but are not limited to inflammatory and immune diseases, such as, diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease, Alzheimers disease and other neurodegenerative diseases, tumors and cancers.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animals, including a mammal, such as a human being.

As used herein, a patient refers to a human subject.

As used herein, an activity refers to a function or functioning or changes in or interactions of a biomolecule, such as polypeptide. Exemplary, but not limiting of such activities are: complexation, dimerization, multimerization, receptor-associated kinase activity or other enzymatic or catalytic activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. an activity can be assessed by assays described herein and by any suitable assays known to those of skill in the art, including, but not limited to in vitro assays, including cell-based assays, in vivo assays, including assays in animal models for particular diseases.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein-protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules of the same type, such as two molecules of a receptor. Dimerization includes homodimerization where two identical molecules interact. Dimerization also includes heterodimerization of two different molecules, such as two subunits of a receptor and dimerization of two different receptor molecules. Typically, dimerization involves two molecules that interact with each other through interaction of a dimerization domain contained in each molecule.

As used herein, in silico refers to research and experiments performed using a computer. In silico methods include, but are not limited to, molecular modeling studies, biomolecular docking experiments, and virtual representations of molecular structures and/or processes, such as molecular interactions.

As used herein, biological sample refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or to sample that is processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived thereform. Also included are soil and water samples and other environmental samples, viruses, bacteria, fungi algae, protozoa and components thereof.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, a biomolecule is any compound found in nature, or derivatives thereof. Exemplary biomolecules include but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Polynucleotides can include nucleotide analogs, include, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, synthetic, with reference to, for example, a synthetic nucleic acid molecule or a synthetic gene refers to a nucleic acid molecule that is produced by recombinant methods and/or by chemical synthesis methods.

As used herein, production by recombinant means by using recombinant DNA methods means the use of the well-known methods of molecular biology for expressing proteins encoded by cloned DNA.

As used herein, “isolated,” with reference to molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated.” Likewise, a molecule that has been purified, partially or substantially, from a native source or recombinant host cell, or produced by synthetic methods, is considered “isolated.” Depending on the intended application, an isolated molecule can be present in any form, such as in an animal, cell or extract thereof; dehydrated, in vapor, solution or suspension; or immobilized on a solid support.

As used herein, the term “vector” refers to a nucleic acid molecule capable of transporting another nucleic acid to which it has been linked. One type of vector is an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors.” In general, expression vectors often are in the form of “plasmids,” which are generally circular double stranded DNA loops that, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, “transgenic animal” refers to any animal, generally a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule can be stably integrated within a chromosome, i.e., replicate as part of the chromosome, or it can be extrachromosomally replicating DNA. In the typical transgenic animals, the transgene causes cells to express a recombinant form of a protein.

As used herein, a reporter gene construct is a nucleic acid molecule that includes a nucleic acid encoding a reporter operatively linked to a transcriptional control sequences. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene,” can be expressed as a fusion protein with a protein of interest or under to the control of a promoter of interest.

As used herein, the phrase “operatively linked” in reference to nucleic acid sequences generally means the nucleic acid molecules or segments thereof are covalently joined into one piece of nucleic acid such as DNA or RNA, whether in single or double stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control or regulatory sequences on one segment control permit expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, the term “operatively linked” in reference to polypeptide sequences, for example, when used in the context of the phrase “at least one domain of a cell surface receptor operatively linked to at least one amino acid encoded by an intron of a gene encoding a cell surface receptor,” means that the amino acids of a domain from a cell surface receptor are covalently joined to amino acids encoded by an intron from a cell surface receptor gene. Hence, a polypeptide that contains at least one domain of a cell surface receptor operatively linked to at least one amino acid encoded by an intron of a gene encoding a cell surface receptor can be an intron fusion protein. It contains one or more amino acids that are not found in a wildtype or predominant form of the receptor. These one or more amino acids are encoded by an intron sequence of the gene encoding the cell surface receptor. Nucleic acids encoding such polypeptides can be produced when an intron sequence is spliced or otherwise covalently joined in-frame to an exon sequence that encodes a domain of a cell surface receptor. Translation of the nucleic acid molecule produced a polypeptide where the amino acid(s) of the intron sequence are covalently joined to a domain of the cell surface receptor.

As used herein, the phrase “generated from a nucleic acid” in reference to the generating of a polypeptide, such as an isoform and intron fusion protein, includes the literal generation of a polypeptide molecule and the generation of an amino acid sequence of a polypeptide from translation of the nucleic acid sequence into a sequence of amino acids.

As used herein, conjugate refers to the joining, pairing, or association of two or molecules. For example, two or more polypeptides (or fragments, domains, or active portions thereof) that are the same or different can be joined together, or a polypeptide (or fragment, domain, or active portion thereof) can be joined with a synthetic or chemical molecule or other moiety. The association of two or more molecules can be through direct linkage, such as joining of the nucleic acid sequence encoding one polypeptide with the nucleic acid sequence encoding another polypeptide, or can be indirect such us by noncovalent or covalent coupling of one molecule with another. For example, conjugation of two or more molecules or polypeptides can be achieved by chemical linkage.

As used herein, a chimeric polypeptide refers to a polypeptide that includes the amino acid sequence of all or part of one polypeptide and an amino acid sequence of all or part of another different polypeptide. The amino acid sequence of the different polypeptides can be linked directly or indirectly. A chimeric polypeptide encoded by a single nucleic acid sequence also is termed a fusion protein.

As used herein, a fusion protein refers to a protein created through recombinant DNA techniques and is achieved by operatively linking all or part of the nucleic acid sequence of one gene with all or part of the nucleic acid sequence of another gene. In some cases, a fusion can encode a chimeric protein containing two or more proteins or peptides.

As used herein, multimerization domain refers to a sequence of amino acids that promote stable interaction of a polypeptide molecule with another polypeptide molecule containing the same or different multimerization domain. Generally, a polypeptide is joined directly or indirectly to the multimerization domain. Exemplary multimerization domains include the immunoglobulin constant region (Fc), leucine zippers, hydrophobic regions, hydrophilic regions, compatible protein-protein interaction domains such as, but not limited to an R subunit of PKA and an anchoring domain (AD), a free thiol which forms an intermolecular disulfide bond between the chimeric molecules, and a protuberance-into-cavity (i.e. hole) and a compensatory cavity of identical or similar size.

As used herein, production with reference to a polypeptide refers to expression and recovery of expressed protein (or recoverable or isolatable expressed protein). Factors that can influence the production of a protein include the expression system and host cell chosen, the cell culture conditions, the secretion of the protein by the host cell, and ability to detect a protein for purification purposes. Production of a protein can be monitored by assessing the secretion of a protein, such as for example, into cell culture medium.

As used herein, “improved production” refers to an increase in the production of a polypeptide compared to production of a control polypeptide. For example, production of an isoform fusion protein is compared to a corresponding isoform that is not a fusion protein or that contains a different fusion. For example, the production of an isoform containing a tPA pre/prosequence can be compared to an isoform containing its endogenous signal sequence. Generally, production of a protein can be improved more than, about or at least 1, 1.5, 2, 3, 4, 5, 6, 7, 8, 9, or 10 fold and more. Typically, production of a protein can be improved by 5, 10, 20, 30, 40, 50 fold or more compared to a corresponding isoform that is not an isoform fusion or does not contain the same fusion.

As used herein, secretion refers to the process by which a protein is transported into the external cellular environment or, in the case of gram-negative bacteria, into the periplasmic space. Generally, secretion occurs through a secretory pathway in a cell, for example, in eukaryotic cells this involves the endoplasmic reticulum and golgi apparatus.

As used herein, a “precursor sequence” or “precursor peptide” or “precursor polypeptide” refers to a sequence of amino acids, that is processed, and that occurs at a terminus, typically at the amino terminus, of a polypeptide prior to processing or cleavage. The precursor sequence includes sequences of amino acids that affect secretion and/or trafficking of the linked polypeptide. The precursor sequence can include one or more functional portions. For example, it can include a presequence (a signal polypeptide) and/or a pro sequence. Processing of a polypeptide into a mature polypeptide results in the cleavage of a precursor sequence from a polypeptide. The precursor sequence, when it includes a presequence and a prosequence also can be referred to as a pre/prosequence.

As used herein, a “presequence”, “signal sequence”, “signal peptide”, “leader sequence” or “leader peptide” refers to a sequence of amino acids at the amino terminus of nascent polypeptides, which target proteins to the secretory pathway and are cleaved from the nascent chain once translocated in the endoplasmic reticulum membrane.

As used herein, a prosequence refers to a sequence encoding a propeptide which when it is linked to a polypeptide can exhibit diverse regulatory functions including, but not limited to, contributing to the correct folding and formation of disulfide bonds of a mature polypeptide, contributing to the activation of a polypeptide upon cleavage of the pro-peptide, and/or contributing as recognition sites. Generally, a pro-sequence is cleaved off within the cell before secretion, although it can also be cleaved extracellularly by exoproteases. In some examples, a pro-sequence is autocatalytically cleaved while in other examples another polypeptide protease cleaves a pro-sequence.

As used herein, homologous with reference to a molecule, such as a nucleic acid molecule or polypeptide, from different species that correspond to each other and that are identical or very similar to each other (i.e., are homologs).

As used herein, heterologous with reference to a molecule, such as a nucleic acid or polypeptide, that is unique in activity or sequence. A heterologous molecule can be derived from a separate genetic source or species. For purposes herein, a heterologous molecule is a protein or polypeptide, regardless of origin, other than a CSR isoform, such as for example a RAGE isoform, or allelic variants thereof. Thus, molecules heterologous to a CSR isoform include any molecule containing a sequence that is not derived from, endogenous to, or homologous to the sequence of a CSR isoform. Examples of heterologous molecules of interest herein include secretion signals from a different polypeptide of the same or different species, a tag such as a fusion tag or label, or all or part of any other molecule that is not homologous to and whose sequence is not the same as that of a CSR isoform. A heterologous molecule can be fused to a nucleic acid or polypeptide sequence of interest for the generation of a fusion or chimeric molecule.

As used herein, a heterologous secretion signal refers to a signal sequence from a polypeptide, from the same or different species, that is different in sequence from the signal sequence of a CSR isoform. A heterologous secretion signal can be used in a host cell from which it is derived or it can be used host cells that differ from the cells from which the signal sequence is derived.

As used herein, an endogenous precursor sequence or endogenous signal sequence refers to the naturally occurring signal sequence associated with all or part of a polypeptide. For example, for the exemplary RAGE polypeptide set forth in SEQ ID NO:2, the signal sequence corresponds to amino acids 1-22. The C-terminal boundary of a signal peptide may vary, however, typically by no more than about 5 amino acids on either side of the signal peptide C-terminal boundary. Algorithms are available and known to one of skill in the art to identify signal sequences and predict their cleavage site (see e.g., Chou et al., (2001), Proteins 42:136; McGeoch et al., (1985) Virus Res. 3:271; von Heijne et al., (1986) Nucleic Acids Res. 14:4683).

As used herein, tissue plasminogen activator (tPA) refers to an extrinsic (tissue-type) plasminogen activator having fibrinolytic activity and typically having a structure with five domains (finger, growth factor, kringle-1, kringle-2, and protease domains). Mammalian t-PA includes t-PA from any animals, including humans. Other species include, but are not limited, to rabbit, rat, porcine, non human primate, equine, murine, dog, cat, bovine and ovine tPA. Nucleic acid encoding tPA including the precursor polypeptide(s) from human and non-human species is known in the art.

As used herein, a tPA precursor sequence refers to a sequence of amino residues that includes the presequence and prosequence from tPA (i.e., is a pre/prosequence, see e.g., U.S. Pat. No. 6,693,181 and U.S. Pat. No. 4,766,075). This polypeptide is naturally associated with tPA and acts to direct the secretion of a tPA from a cell. An exemplary precursor sequence for tPA is set forth in SEQ ID NO:327 and encoded by a nucleic acid sequence set forth in SEQ ID NO:326. The precursor sequence includes the signal sequence (amino acids 1-23) and a prosequence (amino acids 24-35). The prosequence includes two protease cleavage site: one after residue 32 and another after residue 35. Exemplary species variants of precursor sequences are forth in any one of SEQ ID NOS: 332-339; exemplary nucleotide and amino acid allelic variants are set forth in SEQ ID NOS:330 or 331.

As used herein, all or a portion of a tPA precursor sequence refers to any contiguous portion of amino acids of a tPA precursor sequence sufficient to direct processing and/or secretion of tPA from a cell. All or a portion of a precursor sequence can include all or a portion of a wildtype or predominant tPA precursor sequence such as set forth in SEQ ID NO:327 and encoded by SEQ ID NO:326, allelic variants thereof set forth in SEQ ID NO: 331, or species variants set forth in SEQ ID NOS:332-339. For example, for the exemplary tPA precursor sequence set forth in SEQ ID NO:327, a portion of a tPA precursor sequence can include amino acids 1-23, or amino acids 24-35, 24-32, or amino acids 33-35, or any other contiguous sequence of amino acids 1-35 set forth in SEQ ID NO:327.

As used herein, active portion a polypeptide, such as with reference to an active portion of an isoform, refers to a portion of polypeptide that has an activity.

As used herein, purification of a protein refers to is the process of isolating a protein, such as from a homogenate, which can contain cell and tissue components, including DNA, cell membrane and other proteins. Proteins can be purified in any of a variety of ways known to those of skill in the art, such as for example, according to their isoelectric points by running them through a pH graded gel or an ion exchange column, according to their size or molecular weight via size exclusion chromatography or by SDS-PAGE (sodium dodecyl sulfate-polyacrylamide gel electrophoresis) analysis, or according to their hydrophobicity. Other purification techniques include, but are not limited to, precipitation or affinity chromatography, including immuno-affinity chromatography, and others and methods that include combination of any of these methods. Furthermore, purification can be facilitated by including a tag on the molecule, such as a his tag for affinity purification or a detectable marker for identification.

As used herein, detection includes methods that permits visualization (by eye or equipment) of a protein. A protein can be visualized using an antibody specific to the protein. Detection of a protein can also be facilitated by fusion of a protein with a tag including an epitope tag or label.

As used herein, a “tag” refers to a sequence of amino acids, typically added to the N- or C-terminus of a polypeptide. The inclusion of tags fused to a polypeptide can facilitate polypeptide purification and/or detection.

As used herein, an epitope tag includes a sequence of amino acids that has enough residues to provide an epitope against which an antibody can be made, yet short enough so that it does not interfere with an activity of the polypeptide to which it is fused. Suitable tag polypeptides generally have at least 6 amino acid residues and usually between about 8 and 50 amino acid residues.

As used herein, a label refers to a detectable compound or composition which is conjugated directly or indirectly to an isoform so as to generate a labeled isoform. The label can be detectable by itself (e.g., radioisotope labels or fluorescent labels) or, in the case of an enzymatic label, can catalyze chemical alteration of a substrate compound composition which is detectable. Non-limiting examples of labels included fluorogenic moieties, green fluorescent protein, or luciferase.

As used herein, a fusion tagged polypeptide refers to a chimeric polypeptide containing an isoform polypeptide fused to a tag polypeptide.

As used herein, expression refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNA). For purposes herein, a protein that is expressed can be retained inside the cells, such as in the cytoplasm, or can be secreted from the cell.

As used herein, a fusion construct refers to a nucleic acid sequence containing coding sequence from one nucleic acid molecule and the coding sequence from nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, typically few than 10, 9, 8, 7, 6 amino acids. The protein product encoded by a fusion construct is referred to as a fusion polypeptide.

As used herein, an isoform fusion protein or an isoform fusion polypeptide refers to a polypeptide encoded by nucleic acid molecule that contain a coding sequence from an isoform, with or without an intron sequence, and a coding sequence that encodes another polypeptide, such as a precursor sequence or an epitope tag. The nucleic acids are operatively linked such that when the isoform fusion construct is transcribed and translated, an isoform fusion polypeptide is produced in which the isoform polypeptide is joined directly or via a linker to another peptide. An isoform polypeptide, typically is linked at the N-, or C-terminus, or both, to one or more other polypeptides peptides.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, the “amino acids,” which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. .§§. 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH2 or to a carboxyl-terminal group such as COOH.

In a peptide or protein, suitable conservative substitutions of amino acids are known to those of skill in this art and generally can be made without altering an activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions may be made in accordance with those set forth in TABLE 2 as follows:

TABLE 2

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, a peptidomimetic is a compound that mimics the conformation and certain stereochemical features of the biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics can be prepared from biologically active compounds by replacing certain groups or bonds that contribute to the undesirable properties with bioisosteres. Bioisosteres are known to those of skill in the art. For example the methylene bioisostere CH2S has been used as an amide replacement in enkephalin analogs (see, e.g., Spatola (1983) pp. 267-357 in Chemistry and Biochemistry of Amino Acids, Peptides, and Proteins, Weinstein, Ed. volume 7, Marcel Dekker, New York). Morphine, which can be administered orally, is a compound that is a peptidomimetic of the peptide endorphin. For purposes herein, cyclic peptides are included among peptidomimetics.

As used herein, “similarity” between two proteins or nucleic acids refers to the relatedness between the amino acid sequences of the proteins or the nucleotide sequences of the nucleic acids. Similarity can be based on the degree of identity and/or homology of sequences and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)).

As used herein, sequence identity compared along the full length of each SEQ ID to the full length of a RAGE isoform refers to the percentage of identity of an amino acid sequence of a RAGE isoform polypeptide along its full-length to a reference polypeptide, designated by a specified SEQ ID, along its full length. For example, if a polypeptide A has 100 amino acids and polypeptide B has 95 amino acids, identical to amino acids 1-95 of polypeptide A, then polypeptide B has 95% identity when sequence identity is compared along the full length of a polypeptide A compared to full length of polypeptide B. Typically, where a RAGE isoform polypeptide or a reference polypeptide is a mature polypeptide lacking a signal sequence, sequence identity is compared along the full length of the polypeptides, excluding the signal sequence portion. For example, if a RAGE isoform lacks a signal peptide but a reference polypeptide contains a signal peptide, comparison along the full length of both polypeptides for determination of sequence identity excludes the signal sequence portion of the reference polypeptide. For example, SEQ ID NO:10 contains a signal peptide corresponding to amino acids 1-22. Thus, when comparing sequence identity of a full length of a RAGE isoform to the full length of a polypeptide set forth in SEQ ID NO:10, amino acids 1-22 of SEQ ID NO:10 are excluded from the analysis. Additionally, where a RAGE isoform or reference polypeptide is a precursor polypeptide containing a signal sequence, sequence identity is compared along the full length of both polypeptides including the signal sequence portion. As discussed below, and known to those of skill in the art, various programs and methods for assessing identity are known to those of skill in the art. For example, a global alignment, such as using the Needleman-Wunsch global alignment algorithm, can be used to find the optimum alignment and identity of two sequences when considering the entire length. High levels of identity, such as 90% or 95% identity, readily can be determined without software.

As used herein, by homologous (with respect to nucleic acid and/or amino acid sequences) means about greater than or equal to 25% sequence homology, typically greater than or equal to 25%, 40%, 60%, 70%, 80%, 85%, 90% or 95% 90% or 95% sequence homology; the precise percentage can be specified if necessary. For purposes herein the terms “homology” and “identity” often are used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two nucleic acid molecules have nucleotide sequences that are at least 60%, 70%, 80%, 85%, 90%, 95%, 96%, 97%, 98% or 99% “identical” or “homologous” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequences. Identity at a level of 90% or more is indicative of the fact that, assuming for exemplification purposes a test and reference polypeptide length of 100 amino acids are compared. No more than 10% (i.e., 10 out of 100) amino acids in the test polypeptide differs from that of the reference polypeptide. Similar comparisons can be made between test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, a polypeptide comprising a specified percentage of amino acids set forth in a reference polypeptide refers to the proportion of contiguous identical amino acids shared between a polypeptide and a reference polypeptide. For example, a RAGE isoform that comprises 70% of the amino acids set forth in a reference polypeptide having a sequence of amino acids set forth in SEQ ID NO:10 means that the reference polypeptide contains at least 103 contiguous amino acids set forth in the amino acid sequence of SEQ ID NO:10.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. Certain nucleic acid molecules can serve as a “probe” and as a “primer.” A primer, however, as a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

B. Receptor for Advanced Glycation Endproducts (RAGE)

Provided herein are isoforms of Receptor for Advanced Glycation Endproducts (RAGE) and methods of preparing RAGE isoforms. The RAGE isoforms differ from the cognate receptors in that there are insertions and/or deletions so the resulting RAGE receptor isoforms exhibit a difference in one or more activities or functions or in structure compared to the cognate receptor. Activities of functions include, but are not limited to, localization, ligand interactions and signal transduction. The RAGE isoforms typically are secreted, not membrane bound, and are selected to modulate the activities of RAGE.

1. RAGE

RAGE is a cell-surface receptor that is a member of the immunoglobulin family. RAGEs interact with a variety of macromolecular ligands. For example, glycated adducts of macromolecules, such as glycated proteins and lipids produced by non-enzymatic glycation interact with RAGEs. These glycated adducts, also known as advanced glycation endproducts (AGEs) accumulate in cells and tissues during the normal aging process. Enhanced and/or accelerated accumulation of AGEs occurs in sites of inflammation, in renal failure, under hyperglycemic conditions and conditions of systemic or local oxidative stress. Accumulation can occur in tissues such as vascular tissues. For example AGEs accumulate as AGE-β2-microglobulin in subjects and patients and subjects with dialysis-related amyloidosis and in vasculature and tissues of diabetes patients and subjects

RAGE can bind to additional ligands including S100/calgranulins, β-sheet fibrils, amyloid β peptide, Aβ, amylin, serum amyloid A, prion-derived peptides and amphoterin. S100/calgranulins are cytokine-like pro-inflammatory molecules. S100 proteins (S100P) participate in calcium dependent regulation and other signal transduction pathways. S100P forms S100A12 and S100B are extracellular and can bind to RAGE. S100Ps are expressed in a restricted pattern that includes expression in placental and esophageal epithelial cells. S100Ps also are expressed in cancer cells, including breast cancer, colon cancer, prostate cancer, and pancreatic adenocarcinoma. Amphoterin is a polypeptide, approximately 30 kDa, that is expressed in the nervous system. It also is expressed in transformed cells such as c6 glioma cells, HL-60 promyelocytes, U937 promomonocyes, HT1080 fibrosarcoma cells and B16 melanoma cells (Hori et al. (1995) J. Bio. Chem. 270:25752-61).

The RAGE gene (SEQ ID NO:325) is composed of 11 exons interrupted by 10 introns. In the exemplary genomic sequence of RAGE provided herein as SEQ ID NO:325, exon 1 includes nucleotides 601-754, including the 5′-untranslated region. The start codon begins at nucleotide position 703. Intron 1 includes nucleotides 755-937; exon 2 includes nucleotides 938-1044; intron 2 includes nucleotides 1145-1174; exon 3 includes nucleotides 1175-1370; intron 3 includes nucleotides 1371-1536; exon 4 includes nucleotides 1537-1601; intron 4 includes nucleotides 1602-1723; exon 5 includes nucleotides 1724-1811; intron 5 includes nucleotides 1812-1901; exon 6 includes nucleotides 1902-2084; intron 6 includes nucleotides 2085-2226; exon 7 includes nucleotides 2227-2357; intron 7 includes nucleotides 2358-2536; exon 8 includes nucleotides 2537-2678; intron 8 includes nucleotides 2679-3292; exon 9 includes nucleotides 3293-3319; intron 9 includes nucleotides 3320-3447; exon 10 includes nucleotides 3448-3574; intron 10 includes nucleotides 3575-3685; and exon 11 includes nucleotides 3686-3957. The stop codon in exon 11 begins at nucleotide position 3749, and the remainder of exon 11 includes the 3′-untranslated region. Following RNA splicing and the removal of the introns, the primary transcript of RAGE contains exons 1-11 and encodes a polypeptide of 404 amino acids (SEQ ID NO:2).

The RAGE polypeptide contains a number of domains. It has a signal peptide located at the N-terminus. For example, in the exemplary full-length RAGE polypeptide set forth herein as SEQ ID NO:2 and encoded by SEQ ID NO:1, the signal peptide is located at amino acids 1-22. RAGE contains a transmembrane domain. In the exemplary full-length RAGE polypeptide set forth herein as SEQ ID NO:2, the transmembrane domain is between amino acids 343 and 363. RAGE also contains three immunoglobulin-like (Ig-like) domains on the N-terminal side from the transmembrane domain. In the exemplary full-length RAGE polypeptide set forth herein as SEQ ID NO:2, the Ig-like domains are located at amino acids 23-116, 124-221 and 227-317. The first of the Ig-like domains (amino acids 23-116 of SEQ ID NO:2) is a variable-type (V-type) Ig-like domain, whereas the other two Ig-like domains are characterized as similar to constant regions (C-type). The V-type Ig-like domain can mediate interaction with ligands, such as AGEs (Kislinger et al. (1999(J. Biol. Chem. 274: 31740-49). The C-terminus of the RAGE protein is intracellular. In the exemplary full-length RAGE polypeptide set forth herein as SEQ ID NO:2, the C-terminus encompasses amino acids 364-404. The C-terminus participates in RAGE-mediated signal transduction (Ding et al. (2005) Neuroscience letters 373:67-72).

RAGE also can be post-translationally modified. For example, RAGE contains cysteines that can participate in disulfide bonding. In the exemplary full-length RAGE polypeptide set forth herein as SEQ ID NO:2, cysteines at positions C₃₈, C₉₉, C₁₄₄, C₂₀₈, C₂₅₉ and C₃₀₁ can participate in disulfide bonding. Potential disulfide bonds include C₃₈-C₉₉, C₁₄₄-C₂₀₈ and C₂₅₉-C₃₀₁. RAGE contains N-glycosylation sites. In the exemplary full-length RAGE polypeptide set forth herein as SEQ ID NO:2, N-glycosylation sites are N₂₅ and N₈₁.

RAGE participates in a variety of biological activities, directly and indirectly. For example, RAGE is localized to the cell membrane. It contains a transmembrane domain. Removal of this domain can result in a soluble receptor that is secreted into the intercellular space. Ligand binding is another function of RAGE. The receptor can bind ligands such as AGEs and remove AGEs. For example, binding of RAGE to AGEs can result in endocytosis or transcytosis of the ligand. RAGE also can bind ligand when the receptor is complexed with another AGE binding protein, the lactoferrin-like AGE binding protein (LF-L). Binding as a complex can stabilize ligand interactions. RAGE, in a soluble form, also can bind to heparin. Binding to heparin can mediate binding of the receptor to the extracellular matrix (ECM) through interactions with heparin sulfate on cell membranes and the ECM.

RAGE also participates in signal transduction pathways. Participation in such pathways can modulate particular cellular responses, including inducing, augmenting, suppressing and preventing such responses. Examples of cellular responses modulated by RAGE include, but are not limited to, induction of neurite outgrowth, cytoskeletal reorganization, cellular oxidant stress induction, NF-κB modulation, triggering and modulation of pro-inflammatory responses, activation of the RAS-MAP kinase pathway, induction of cytokines, induction of growth factors such as VEGF, TNFα, and PDGF, induction of type IV collagen expression, induction of VCAM-1, ERK1/2 phosphorylation, EC migration, modulation of Rac, cdc42, Rho family of proteins and modulation of GTPases. RAGE also can participate in self-regulation and regulation of endogenous RAGE such as by modulating the expression from a RAGE promoter.

C. RAGE Receptor Isoforms

Provided herein are RAGE receptor isoforms and methods of preparing RAGE receptor isoforms. The RAGE receptor isoforms differ from the cognate receptor in that the polypeptide contains insertions and/or deletions of amino acids and the resulting RAGE receptor isoforms exhibit a difference in one or more biological activities or functions or structure compared to the cognate receptor. Such changes to a RAGE receptor polypeptide sequence can include disruption or elimination of all of or a portion of one or more domains of RAGE. For example, the changes that RAGE isoforms exhibit compared to a RAGE include, but are not limited to elimination and/or disruption of all or part of a signal peptide, an immunoglobulin-like domain, a cytosolic domain, and/or a transmembrane domain. In one example, the RAGE isoforms provided herein differ from the full-length RAGE cognate receptor in that the nucleic acids encoding the isoforms retain part or all of any one or more of the ten introns. The RAGE receptor isoforms provided herein can be used for modulating the activity of a cell surface receptor, particularly a RAGE. They also can be used as targeting agents for delivery of molecules, such as drugs or toxins or nucleic acids, to targeted cells or tissues in vivo or in vitro.

Pharmaceutical compositions containing one or more different RAGE isoforms are provided. The pharmaceutical compositions can be used to treat diseases that include inflammatory diseases, immune diseases, cancers, and other diseases that manifest aberrant angiogenesis or neovascularization. Cancers include breast, lung, colon, gastric cancers, pancreatic cancers and others. Inflammatory diseases, include, for example, diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease, Alzheimers disease and other neurodegenerative diseases, and other diseases known to those of skill in the art in which a RAGE, VEGF and other immune response and inflammatory responses are implicated, involved or in which they participate.

Also provided are methods of treatment of diseases and conditions by administering the pharmaceutical compositions or delivering a RAGE isoform, such by administering a vector that encodes the isoform. Administration can be effected in vivo or ex vivo.

Methods are provided herein for expressing, isolating and formulating RAGE isoforms, including producing RAGE isoforms and nucleic acid molecules encoding RAGE isoforms. Also provided are combinations of RAGE isoforms and other cell surface receptor isoforms including, but not limited to herstatins and other ERBB isoforms, isoforms of FGFRs and others.

1. Identification and Production of RAGE Isoforms

As noted, RAGE isoforms are polypeptides that lack a domain or portion of a domain or have a disruption of a domain compared with a wildtype or predominant form of RAGE and can be altered in an activity compared to the cognate receptor. RAGE isoforms represent variants of a RAGE gene that can be generated by alternate splicing or by recombinant or synthetic (e.g., in silico and/or chemical synthesis) methods.

Typically, a RAGE isoform produced from an alternatively spliced RNA is not a predominant form of a polypeptide produced encoded by a gene. In some instances, a RAGE isoform can be a tissue-specific or developmental stage-specific polypeptide or disease specific (i.e., can be expressed at a difference level from tissue-to-tissue or stage-to-stage or in a disease state compared to a non-diseased state or only may be expressed in the tissue, at the stage or during the disease process or progress). Alternatively spliced RNA form that can encode RAGE isoforms include, but are not limited to, exon deletion, exon retention, exon extension, exon truncation, and intron retention alternatively spliced RNAs.

(a) Alternative Splicing and Generation of RAGE Isoforms

Genes in eukaryotes include intron and exons that are transcribed by RNA polymerase into RNA products generally referred to as pre-mRNA. Pre-mRNAs are typically intermediate products that are further processed through RNA splicing and processing to generate a final messenger RNA (mRNA). Typically, a final mRNA contains exons sequences and is obtained by splicing out the introns. Boundaries of introns and exons are marked by splice junctions, sequences of nucleotides that are used by the splicing machinery of the cell as signals and substrates for removing introns and joining together exon sequences. Exons are operatively linked together to form a mature RNA molecule. Typically, one or more exons in an mRNA contains an open reading frame encoding a polypeptide. In many cases, an open reading frame can be generated by operatively linking two or more exons; for example, a coding sequence can span exon junctions and an open reading frame is maintained across the junctions.

RNA also can undergo alternative splicing to produce a variety of different mRNA transcripts from a single gene. Alternatively spliced mRNAs can contain different numbers of and/or arrangements of exons. For example, a gene that has 10 exons can generate a variety of alternatively spliced mRNAs. Some mRNAs can contain all 10 exons, some with only 9, 8, 7, 6, 5 etc. In addition, products for example, with 9 of the 10 exons, can be among a variety of mRNAs, each with a different exon missing. Alternatively spliced mRNAs can contain additional exons, not typically present in an RNA encoding a predominant or wild type form. Addition and deletion of exons includes addition and deletion, respectively of a 5′ exon, 3′exon and an exon internal in an RNA. Alternatively spliced RNAs also include addition of an intron or a portion of an intron operatively linked to or within an RNA. For example, an intron normally removed by splicing in an RNA encoding a wildtype or predominant form can be present in an alternatively spliced RNA. An intron or intron portion can be operatively linked within an RNA, such as between two exons. An intron or intron portion can be operatively linked at one end of an RNA, such as at the 3′ end of a transcript. In some examples, the presence of intron sequence within an RNA terminates transcription based on poly-adenylation sequences within an intron.

Alternative RNA splicing patterns can vary depending upon the cell and tissue type. Alternative RNA splicing also can be regulated by developmental stage of an organism, cell or tissue type. For example, RNA splicing enzymes and polypeptides that regulate RNA splicing can be present at different concentrations in particular cell and tissue types and at particular stages of development. In some cases, a particular enzyme or regulatory polypeptide can be absent from a particular cell or tissue type or at particular stage of development. These differences can produce different splicing patterns for an RNA within a cell or tissue type or stage, thus giving rise to different populations of mRNAs. Such complexity can generate a number of protein products appropriate for particular cell types or developmental stages.

Alternatively spliced mRNAs can generate a variety of different polypeptides, also referred to herein as isoforms. Such isoforms can include polypeptides with deletions, additions and shortenings. For example, a portion of an open reading normally encoded by an exon can be removed in an alternatively spliced mRNA, thus resulting in a shorter polypeptide. An isoform can have amino acids removed at the N or C terminus or the deletion can be internal. An isoform can be missing a domain or a portion of a domain as a result of a deleted exon. Alternatively spliced mRNAs also can generate polypeptides with additional sequences. For example, a stop codon can be contained in an exon; when this exon is not included in an mRNA, the stop codon is not present and the open reading frame continues into the sequences contained in downstream exons. In such example, additional open reading frame sequences add additional amino acid sequence to a polypeptide and can include addition of a new domain or a portion thereof.

(i) Isoforms Generated by Intron Modification

Among the RAGE isoforms that can be generated by alternate RNA splicing patterns are isoforms generated through intron modification. In one example, a RAGE isoform is generated by alternative splicing such that one or more introns are retained compared to an mRNA transcript encoding a wildtype or predominant form of RAGE. The retention of one or more intron sequences can generate transcripts encoding RAGE isoforms that are shortened compared to a wildtype or predominant form of RAGE. A retained intron sequence can introduce a stop codon in the transcript and thus prematurely terminate the encoded polypeptide. A retained intron sequence also can introduce additional amino acids into a RAGE polypeptide, such as the insertion of one or more codons into a transcript such that one or more amino acids are inserted into a domain of RAGE. Intron retention includes the inclusion of a full or partial intron sequence into a transcript encoding a RAGE isoform. The retained intron sequence can introduce nucleotide sequence with codons in-frame to the surrounding exons or it can introduce a frame shift into the transcript. Exemplary nucleotide sequences of intron retention transcripts include SEQ ID NOS:5-9.

(ii) Isoforms Generated by Exon Modifications

RAGE isoforms can be generated by modification of an exon relative to a corresponding exon of an RNA transcript encoding a wildtype or predominant form of a RAGE polypeptide. Exon modifications include alternatively spliced RNA forms such as exon truncations, exon extensions, exon deletions and exon insertions. These alternatively spliced RNAs can encode RAGE isoforms which differ from a wildtype or predominant form of a RAGE polypeptide by including additional amino acids and/or by lacking amino acid sequences present in a wildtype or predominant form of a RAGE polypeptide.

Exon insertions are alternative spliced RNAs that contains at least one exon not typically present in an RNA encoding a wildtype or predominant form of a polypeptide. An inserted exon can operatively link additional amino acids encoded by the inserted exon to the other exons present in an RNA. An inserted exon also can contain one or more stop codons such that the RNA encoded polypeptide terminates as a result of such stop codons. If an exon containing such stop codons is inserted upstream of an exon that contains the stop codon used for polypeptide termination of a wildtype or predominant form of a polypeptide, a shortened polypeptide can be produced.

An inserted exon can maintain an open reading frame, such that when the exon is inserted, the RNA encodes an isoform containing an amino acid sequence of a wildtype or predominant form of a polypeptide with additional amino acids encoded by the inserted exon. An inserted exon can be inserted 5′, 3′ or internally in an RNA, such that additional amino acids encoded by the inserted exon are linked at the N terminus, C-terminus or internally, respectively in an isoform. An inserted exon also can change the reading frame of an RNA in which it is inserted, such that an isoform is produced that contains only a portion of the sequence of amino acids in a wildtype or predominant form of a polypeptide. Such isoforms can additionally contain amino acid sequence encoded by the inserted exon and also can terminate as a result of a stop codon contained in the inserted exon.

RAGE isoforms also can be produced from exon deletion events. An exon deletion refers to an event of alternative RNA splicing that produces a nucleic acid molecule that lacks at least one exon compared to an RNA encoding a wildtype or predominant form of a polypeptide. Deletion of an exon can produce a polypeptide of alternate size such as by removing sequences that encode amino acids as well as by changing the reading frame of an RNA encoding a polypeptide. An exon deletion can remove one or more amino acids from an encoded polypeptide; such amino acids can be N-terminal, C-terminal or internal to a polypeptide depending upon the location of the exon in an RNA sequence. Deletion of an exon in an RNA also can cause a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide. In one example, a shift of reading frame produces an isoform that is shortened compared to a wildtype or predominant form of a polypeptide. Such shortened isoforms also can contain sequences of amino acids not present in a wildtype or predominant form of a polypeptide.

RAGE isoforms also can be produced by exon extension in an RNA. Exon extension is an event of alternative RNA splicing that produces a nucleic acid molecule that contains at least one exon that is greater in length (number of nucleotides contained in the exon) than the corresponding exon in an RNA encoding a wildtype or predominant form of a polypeptide. Additional sequence contained in an exon extension can encode additional amino acids and/or can contain a stop codon that terminates a polypeptide. An exon insertion containing an in-frame stop codon can produce a shortened isoform, that terminates in the sequence of the exon extension. An exon insertion also can shift the reading frame of an RNA, resulting in an isoform containing one or more amino acids not present in a wildtype or predominant form of a polypeptide and/or an isoform that terminates at a sequence different from that of a wildtype or predominant form of a polypeptide. An exon extension can include sequences contained in an intron of an RNA encoding a wildtype or predominant form of a polypeptide.

RAGE isoforms also can be produced by exon truncation. Exon truncations are RNA molecules that contain a shortening of one or more exons such that the one or more exons are shorter in length (number of nucleotides) compared to a corresponding exon in an RNA encoding a wildtype or predominant form of a polypeptide. An RNA molecule with an exon truncation can produce a polypeptide that is shortened compared to a wildtype or predominant form of a polypeptide. An exon truncation also can result in a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide.

Alternatively spliced RNAs including exon modifications can produce RAGE isoforms that a lack a domain or a portion thereof and can produce RAGE isoforms that are reduced in or lack a biological activity. For example, exon modified RNAs can encode shortened RAGE polypeptides that lack a domain or portion thereof. Exon modified RNAs also can encode polypeptides where a domain is interrupted by inserted amino acids and/or by a shift in reading frame that interrupts a domain with one or more amino acids not present in a wildtype or predominant form of a polypeptide.

(b) In Silico Generated RAGE Isoforms

RAGE isoforms can be generated by in silico methods and synthetic and/or recombinant production to produce polypeptides that are modified compared to a wildtype or predominant form of a polypeptide. Typically, such RAGE isoforms have a modified sequence compared to a wildtype or predominant form. For example, RAGE isoforms are generated that are truncated. These truncated forms can have deletions internally, at the N-terminus, at the C-terminus or a combination thereof. RAGE isoforms also include lengthened forms that have additional amino acids internally, at the N-terminus, at the C-terminus or a combination thereof. For example, as is described further herein, by using available software programs, intron and exons, structures and encoded protein domains can be identified in a nucleic acid, such as a RAGE gene. Recombinant nucleic acid molecules encoding polypeptides can be synthesized that contain one or more exons and an intron or portion thereof. For example, recombinant molecules can contain one or more amino acids and/or a stop codon encoded by an intron, operatively linked to an exon, producing an isoform that has a modified intron-exon structure compared to a wildtype or predominant form of RAGE.

2. RAGE Isoform Polypeptide Structure

The exemplary RAGE gene (see e.g., SEQ ID NO:325) includes 11 exons that contain protein coding sequence interrupted by 10 introns. In a wildtype or predominant form of RAGE such as set forth in SEQ ID NO:2, encoded by the nucleotide sequence set forth as SEQ ID NO:1, eleven exons are joined by RNA splicing to form a transcript encoding a 404 amino acid polypeptide that includes a signal sequence, three Ig-like domains, a transmembrane domain and a cytosolic domain. RAGE isoforms such as those described herein, can be generated by alternate splicing such that the splicing pattern of the RAGE is altered compared to the transcript encoding a wildtype or predominant form of RAGE.

A RAGE isoform includes receptor isoforms that lacks a domain or portion of a domain or that has a disruption in a domain such as by the insertion of one or more amino acids compared to a wildtype or predominant form of a RAGE receptor polypeptide. RAGE isoforms can contain a new domain and/or a function compared to a wildtype and/or predominant form of the receptor. The deletion, disruption and or insertion in the polypeptide sequence of a RAGE isoform is sufficient to alter an activity compared to that of a RAGE or change the structure compared to the RAGE, such as by elimination of one or more domains or by addition of a domain or portion thereof, such as one encoded by an intron in the RAGE gene.

RAGE isoforms can lack one or more domains or part of one or more domains compared to the polypeptide sequence of a wildtype or predominant form of the receptor. For example, a RAGE isoform can lack the cytosolic domain or part of the cytosolic domain. Such isoforms can lack some or all of amino acids set forth as amino acids 364-404 of SEQ ID NO:2. Exemplary RAGE isoforms lacking a cytosolic domain include SEQ ID NOs: 10-14. A RAGE isoform can lack the transmembrane domain or part of the transmembrane domain. Such isoforms can lack some or all of amino acids set forth as amino acids 343-363 of SEQ ID NO:2 Exemplary RAGE isoforms lacking a transmembrane domain include SEQ ID NOs: 10-14. A RAGE isoform can lack all or part of an Ig-like domain. In one example, an isoform lacks all or part of a C-type Ig-like domain. Such isoforms can include isoforms that lack the second and/or third Ig-like domains of the RAGE receptor (the two C-type domains). A RAGE isoform can lack part of the second Ig-like domain, all of the second Ig-like domain, part of the third Ig-like domain or all of the third Ig-like domain or a combination thereof. Exemplary RAGE isoforms lacking part or all of a C-type Ig-like domain include SEQ ID NOs: 10, 11, 13 and 14. A RAGE isoform also can lack part of or all of a V-type Ig-like domain.

A RAGE isoform can include a disruption in a domain such as by the insertion of one or more amino acids compared to the polypeptide sequence of a wildtype or predominant form of RAGE. For example, a RAGE isoform can include an insertion of one or more amino acids in the signal peptide, in a V-type Ig-like domain, in one or both of the C-type Ig-like domains, in the transmembrane domain and/or in the cytosolic domain. An exemplary RAGE isoform that contains an insertion of amino acids in a C-type Ig-like domain is set forth as SEQ ID NO: 13.

RAGE isoforms also can include RAGE polypeptide sequences that include the addition of a domain or a partial domain into the sequence. For example, a RAGE isoform can include the addition of amino acids at the C-terminus of the protein, where such amino acid sequence is not found in the wildtype and/or predominant form of RAGE. In one example, the additional amino acids can be intron-encoded amino acids due to the presence of a retained intron with the nucleic acid sequence of an isoform. Exemplary RAGE isoforms that include additional amino acid sequences at the C-terminal end of the polypeptide sequence include SEQ ID NOs: 10, 12, 13 and 14.

RAGE polypeptides also contain amino acids that are not formally part of a domain but are found in between designated domains (referred to herein as linking regions). RAGE isoforms also can include insertion, deletion and/or disruption in one or more linking regions. Exemplary RAGE isoforms include SEQ ID NOS: 10-14.

3. RAGE Isoform Biological Activities

One or more biological activities can be altered in a RAGE isoform compared with a wildtype or predominant form of RAGE. Altered biological activities can include localization of the receptor, interaction with one or more ligands and/or altered signal transduction.

In one example, a RAGE isoform is altered in localization. For example, an isoform is lacking all of or part of the transmembrane domain of RAGE or has an insertion of one or more amino acids in the transmembrane domain of RAGE. Such isoforms can be altered in localization such that the isoform is not embedded in the membrane. For example, an isoform can be secreted extracellularly. It may be soluble and found for example intercellular spaces. Such isoforms also can be associated with the extracellular portion of the membrane or ECM such as through heparin sulfate binding.

In one example, a RAGE isoform is altered in ligand interaction. For example, an isoform is reduced in binding affinity for one or more ligands. In another example, an isoform is increased in affinity for one or more ligands. An isoform also can be altered in specificity of ligand binding. For example, an isoform can bind one ligand preferentially over other ligands, where such preferential binding is in comparison to the ligand specificity of a wildtype or predominant form of RAGE. A RAGE isoform can be altered in ligand endocytosis and/or transcytosis. A RAGE isoform also can be altered in its interaction with LF-L such that ligand binding is altered. Isoforms altered in ligand interaction can include isoforms that lack all or part of a V-type Ig-like domain or have a disruption of a V-type Ig-like domain. RAGE isoforms altered in ligand interaction also can include isoforms that have a conformational change compared to a wildtype or predominant form of RAGE.

RAGE isoforms can be altered in one or more facets of signal transduction. An isoform, compared with a wildtype or predominant form of RAGE can be altered in the modulation of one or more cellular responses, including inducing, augmenting, suppressing and preventing cellular responses to ligand, environmental conditions and other stimuli. Examples of cellular responses that can be altered in a RAGE isoform, include, but are not limited to, induction of neurite outgrowth, cytoskeletal reorganization, cellular oxidant stress induction, NF-κB modulation, triggering and modulation of pro-inflammatory responses, activation of the RAS-MAP kinase pathway, induction of cytokines, induction of growth factors such as VEGF, TNFα, and PDGF, induction of type IV collagen expression, induction of VCAM-1, ERK1/2 phosphorylation, EC migration, modulation of Rac, cdc42, Rho family of proteins, modulation of GTPases and modulating the expression from a RAGE promoter.

Generally, an activity is altered in an isoform at least 0.1, 0.5, 1, 2, 3, 4, 5, or 10 fold compared to a wildtype and/or predominant form of the receptor. Typically, an activity is altered 10, 20, 50, 100 or 1000 fold or more. For example, an isoform can be reduced in an activity compared to a wildtype and/or predominant form of the receptor. An isoform also can be increased in an activity compared to a wildtype and/or predominant form of a receptor. In assessing an activity of a RAGE isoform, the isoform can be compared with a wildtype and/or predominant form of RAGE. For example, a RAGE isoform can be altered in an activity compared to the RAGE polypeptide set forth as SEQ ID NO:2.

a. Negatively Acting and Inhibitory Isoforms

RAGE isoforms also can modulate an activity of another RAGE polypeptide. The modulated polypeptide can be a wildtype or predominant form of RAGE. A RAGE isoform also can modulate another RAGE isoform, such as a RAGE isoform expressed in a disease or condition. A RAGE isoform can interact directly or indirectly to modulate an activity a RAGE polypeptide. Such RAGE isoforms can act as negatively acting ligands by preventing or inhibiting one or more biological activities of a wildtype or predominant form of RAGE. A negatively acting ligand need not bind or affect the ligand binding domain of a receptor, nor affect ligand binding of the receptor.

A RAGE isoform also can indirectly modulate the activity of another RAGE form. In one example, a RAGE isoform competes with another RAGE form for ligand. Such isoforms can thus bind ligand and reduce the amount of ligand available to bind to other RAGE polypeptides. RAGE isoforms that bind and compete for one or more ligands of RAGE can include RAGE isoforms that do not participate in signal transduction or are reduced in their ability to participate in signal transduction compared to a cognate RAGE.

A negatively acting RAGE isoform that competes for ligand can include a ligand binding domain such as an Ig-like V-type domain of a cognate RAGE receptor. A negatively acting RAGE isoform can lack one or more domains, such that the isoform although bound to ligand does not modulate signal transduction. For example, such isoforms can lack an intracellular C-terminal domain. In one example, a RAGE isoforms lacks one or more amino acids of the C-terminal domain of the cognate receptor, for example, lacking one or more amino acids corresponding to amino acids 364-404 of the RAGE polypeptide set forth as SEQ ID NO:2. A dominant negative RAGE isoform also can lack all or part of a transmembrane domain. In one example, a RAGE isoforms lacks one or more amino acids corresponding to the transmembrane of the cognate receptor set forth as SEQ ID NO:2, such as one or more amino acids between amino acids 343-363 of SEQ ID NO:2. A negatively acting RAGE isoform can lack a part or all of one or more Ig-like type C domains. For example, a RAGE isoform lacks one or more amino acids or contains a disruption of the Ig-like C-type domain of the wildtype and/or predominant form of the RAGE receptor set forth as SEQ ID NO:2, corresponding to amino acids 124-221 and 227-317.

D. Methods for Identifying and Generating RAGE Isoforms

RAGE isoforms can be identified and produced by any of a variety of methods. For example, RAGE isoforms can be generated by analysis and identification of genes and expression products (RNAs) using cloning methods in combination with bioinformatics methods such as sequence alignments and domain mapping and selections.

Provided herein are methods for identifying and isolating RAGE isoforms that utilize cloning of expressed gene sequences and alignment with a gene sequence such as a genomic DNA sequence. Expressed sequences, such as cDNAs or regions of cDNAs, are isolated. Primers can be designed to amplify a cDNA or a region of a cDNA. In one example, primers are designed which overlap or flank the start codon of the open reading frame of a RAGE gene and primers are designed which overlap or flank the stop codon of the open reading frame. Primers can be used in PCR, such as in reverse transcriptase PCR (RT-PCR) with mRNA, to amplify nucleic acid molecules encoding open reading frames. Such nucleic acid molecules can be sequenced to identify those that encode an isoform. In one example, nucleic acid molecules of different sizes (e.g. molecular masses) from a predicted size (such as a size predicted for encoding a wildtype or predominant form) are chosen as candidate isoforms. Such nucleic acid molecules then can be analyzed, such by a method described herein, to further select isoform-encoding molecules having specified properties.

Computational analysis is performed using the obtained nucleic acid sequences to further select candidate isoforms. For example, cDNA sequences are aligned with a genomic sequence of a selected candidate gene. Such alignments can be performed manually or by using bioinformatics programs such as SIM4, a computer program for analysis of splice variants. Sequences with canonical donor-acceptor splicing sites (e.g. GT-AG) are selected. Molecules can be chosen which represent alternatively spliced products such as exon deletion, exon retention, exon extension and intron retention can be selected.

Sequence analysis of isolated nucleic acid molecules also can be used to further select isoforms that retain or lack a domain and/or a function compared to a wildtype or predominant form. For example, isoforms encoded by isolated nucleic acid molecules can be analyzed using bioinformatics programs such as described herein to identify protein domains. Isoforms then can be selected which retain or lack a domain or a portion thereof.

In one embodiment, isoforms are selected that lack a transmembrane domain or portion thereof sufficient to reduce or abolish membrane localization. For example, isoforms are selected that lack one or more amino acids of the transmembrane domain or have a disruption of the transmembrane domain such as an insertion of one or more amino acids. Such isoforms also can lack a cytosolic domain at the C-terminus of the receptor or have an altered C-terminal sequence compared to a wildtype or predominant form of RAGE. Isoforms also can be selected that lack a transmembrane domain or portion thereof and have one or more amino acids operatively linked in place of the missing domain or portion of a domain. Such isoforms can be the result of alternative splicing events such as exon extension, intron retention, exon deletion and exon insertion. In some cases, such alternatively spliced RNAs alter the reading frame of an RNA and/or operatively link sequences not found in an RNA encoding a wildtype or predominant form.

In another embodiment, isoforms lack at least one Ig-like domain or part of an Ig-like domain. For example, an isoform is selected that lacks a C-type Ig-like domain. Such isoforms can include those that lack one or more amino acids of the Ig-like domain closest to the C-terminus of RAGE. For example, RAGE isoforms can lack one or more of amino acids corresponding to amino acids 227-317 of SEQ ID NO:2. In one example, an isoform lacks a transmembrane domain and lacks all or part of the Ig-like domain closest to the C-terminus of RAGE. In another example, a RAGE isoform lacks all or part of both C-type Ig-like domains. Such isoforms also can lack a transmembrane domain. The isoforms can be the result of alternative splicing events such as exon extension, intron retention, exon deletion and exon insertion. In some case, such alternatively spliced RNAs alter the reading frame of an RNA and/or operatively link sequences not found in an RNA encoding a wildtype or predominant form. Such isoforms can include additional amino acid sequences not found in a wildtype or predominant form of RAGE. For example, additional amino acids can include intron-encoded amino acids. In one example, additional amino acid sequence is contained at the C-terminus of a RAGE isoform.

Nucleic acid molecules can be selected which encode A RAGE isoform and have an activity that differs from a wildtype or predominant form of RAGE. In one example, RAGE isoforms are selected that lack a transmembrane domain such that the isoforms are not membrane localized and are secreted from a cell. In another example, RAGE isoforms are selected that lack all or part of at least one Ig-like domain and that are altered in one or more biological activities including ligand interactions and signal transduction.

E. Exemplary RAGE Isoforms

Provided herein are exemplary RAGE isoforms that have an altered domain organization compared to a cognate RAGE due to the retention of an intron-encoded sequence in the nucleic acid molecule that encodes the RAGE isoform. Provided herein are exemplary RAGE isoforms that lack one or more domains or parts of domains of RAGE.

RAGE isoforms provided herein are encoded by nucleic acid molecules that include all or a portion of any one or more introns of RAGE, operatively linked to an exon. The intron portion can include one codon, including a stop codon, which results in RAGE isoform that ends at the end of the exon, or can include more codons so that the RAGE isoform includes intron encoded residues.

In the exemplary genomic sequence of RAGE set forth in SEQ ID NO:325, such introns include intron 1 containing nucleotides 755-937, intron 2 containing nucleotides 1045-1174, intron 3 containing nucleotides 1371-1536, intron 4 containing nucleotides 1602-1723, intron 5 containing nucleotides 1812-1901, intron 6 containing nucleotides 2085-2226, intron 7 containing nucleotides 2358-2536, intron 8 containing nucleotides 2779-3292, intron 9 containing nucleotides 3320-3447, and intron 10 containing nucleotides 3575-3685. An intron-encoded portion of an isoform can exist N-terminally, C-terminally, or internally to an exon sequence(s) operatively linked to the intron. An isoform includes intron-encoded amino acids from any one or more of introns 1, 2, 3, 4, 5, 6, 7, 8, 9, or 10 internally within the isoform, or at the N- or C-terminus or the isoform is truncated at the end of an exon.

Among the RAGE isoforms provided herein is isoform A05 set forth as SEQ ID NO:10, and the encoding nucleic acid sequence set forth as SEQ ID NO: 5. Clone A05 contains 928 bases, including an intron portion encoding the C-terminal-portion of the RAGE polypeptide. The intron portion contains the first 92 nucleotides of intron 3. The intron 3 portion encodes twenty eight amino acids followed by a stop codon. In the clone this portion is operatively linked to an open reading frame of exons 1-3. The encoded RAGE isoform is truncated compared to the cognate RAGE and includes the twenty eight additional intron encoded amino acids at the C-terminus. A05 is a 146 amino acid polypeptide. It contains a signal sequence at the N-terminus at amino acids 1-22 and an Ig-like V-type domain following the signal sequence at amino acids 23-116. The A05 RAGE isoform lacks both C-type Ig like domains compared to a cognate RAGE such as set forth in SEQ ID NO:2. It also lacks a transmembrane domain and also the C-terminal amino acids found in the cognate receptor. Isoform A05 includes an additional (i.e. intron-encoded) 28 amino acids at the C-terminus of the polypeptide not present in the cognate RAGE set forth as SEQ ID NO: 2.

Provided herein is an exemplary RAGE isoform C02, set forth as SEQ ID NO:13, and encoded by a nucleic acid sequence set forth as SEQ ID NO:8. Clone C02 contains 994 bases, including two intron portions encoding portions of the RAGE isoform polypeptide. The first intron portion contains the first 48 nucleotides of intron 4. The intron 4 portion encodes sixteen amino acids that are not present in the cognate RAGE. Thus, the resultant polypeptide contains an insertion of sixteen amino acids within the Ig-like C1 domain. In the clone this portion is operatively linked between an open reading frame of exons 1-4 and exon 5. In addition, clone C02 also contains a second intron portion which contains the first 75 nucleotides of intron 6. The intron 6 portion encodes twenty amino acids followed by a stop codon. In the clone this portion is operatively linked to an open reading frame following exon 6. The encoded RAGE isoform is truncated compared to the cognate RAGE and includes the twenty additional intron encoded amino acids at the C-terminus. C02 contains 266 amino acids. This isoform includes an N-terminal signal sequence at amino acids 1-22, followed by a V-type Ig-like domain at amino acids 23-116 and one C-type Ig-like domain at amino acids 124-237. It lacks a second C-type Ig-like domain except for the first 4 amino acids (amino acids 243-246) corresponding to amino acids 227-230 of SEQ ID NO:2. In addition, the first C-type Ig-like domain included in C02 contains a disruption. An additional (i.e. intron encoded) 16 amino acids are inserted; these 16 amino acids are positions 142-157 of SEQ ID NO:13. The insertion point for these amino acids corresponds to amino acids 141-142 of SEQ ID NO:2. C02 isoform contains an additional (i.e. intron encoded) 20 amino acids at the C-terminus of the polypeptide, amino acids 247-266 not present in the cognate RAGE.

Another exemplary RAGE isoform encoded by clone C06 is provided. The encoding nucleic acid sequence is set forth in SEQ ID NO: 7, and encodes a polypeptide having a sequence of amino acids set forth in SEQ ID NO:12. Clone C06 contains 1165 bases, including an intron portion encoding the C-terminal-portion of the RAGE polypeptide. The intron portion contains the first 201 nucleotides of intron 8. The intron 8 portion encodes sixty six amino acids followed by a stop codon. In the clone this portion is operatively linked to an open reading frame of exons 1-8. The encoded RAGE isoform is truncated compared to the cognate RAGE and includes the sixty six additional intron encoded amino acids at the C-terminus. Isoform C06, set forth as SEQ ID NO:12, is a RAGE isoform that is 387 amino acids in length and contains an N-terminal signal sequence at amino acids 1-22, a V-type Ig-like domains at amino acids 23-116 and two C-type Ig-like domains (amino acids 124-221 and amino acids 227-317). This isoform lacks a transmembrane domain and the amino acids present at the C-terminus of cognate RAGE. Compared with a cognate RAGE set forth in SEQ ID NO:2, the C06 isoform contains a deletion of amino acids 322-404. C06 isoform contains an additional (i.e. intron encoded) 66 amino acids following the second C-type Ig-like domain (amino acids 322-387) that are not found in the cognate RAGE.

Another exemplary RAGE isoform, F06, is set forth as SEQ ID NO:11, and is encoded by a nucleic acid sequence set forth as SEQ ID NO:6. Clone F06 contains 941 bases, including an intron portion at the C-terminus containing the first 24 nucleotides of intron 5. The intron 5 portion encodes a stop codon that is operatively linked with an open reading frame of exons 1-5 of the encoded polypeptide thereby resulting in a RAGE isoform that is truncated compared to a cognate RAGE. The F06 isoform is 172 amino acids in length, including the signal sequence. The F06 isoform contains an N-terminal signal sequence at amino acids 1-22, and a V-type Ig-like domain at amino acids 23-116. It contains part of the first C-type Ig-like domain, amino acids 124-172, corresponding to amino acids 124-172 of SEQ ID NO:2. Additionally, it can contains an amino acid replacement of glutamic acid for valine at the position corresponding to amino acid 171 of SEQ ID NO:2. F06 isoform lacks a second C-type Ig-like domain, a transmembrane domain and the C-terminal cytosolic domain.

Also provided herein is an exemplary RAGE isoform C08 having a nucleic acid sequence set forth in SEQ ID NO:9 and encoding a 173 amino acid polypeptide set forth as SEQ ID NO:14. Clone C08 contains 1415 bases, including three intron portions encoding portions of the RAGE isoform polypeptide. The first intron portion includes the entire sequence of intron 4 operatively linked between the open reading frame of exons 1-4 and exon 5. The intron 4 portion encodes thirty two amino acids followed by a stop codon, resulting in a RAGE isoform that is truncated compared to a cognate RAGE. Thus, the remainder of the nucleotide sequence of clone C08 is non-coding, but contains retained intron 6 and intron 9 sequences. The 173 amino acid sequence of clone C08 contains an N-terminal signal sequence at amino acids 1-22 and a V-type Ig-like domain at amino acids 23-116. Isoform C08 contains part of the first C-type Ig-like domain corresponding to amino acids 124-141 of SEQ ID NO:2. C08 isoform lacks a second C-type Ig-like domain and a transmembrane domain. It also does not contain the C-terminal amino acids corresponding to amino acids 364-404 of SEQ ID NO:2. Isoform C08 has an additional 32 amino acids at its C-terminus, amino acids 142-174 that are not found in the cognate RAGE.

1. Allelic Variants of Isoforms

Allelic variants and species variants of RAGE isoforms can be generated or identified. Such variants differ in one or more amino acids from a particular RAGE isoform or cognate RAGE. Allelic variation occurs among members of a population and species variation occurs between species. For example, isoforms can be derived from different alleles of a gene; each allele can have one or more amino acid differences from the other. Such alleles can have conservative and/or non-conservative amino acid differences. Allelic variants also include isoforms produced or identified from different subjects, such as individual subjects or animal models or other animals. Amino acid changes can result in modulation of an isoform biological activity. In some cases, an amino acid difference can be “silent,” having no or virtually no detectable effect on a biological activity. Allelic variants of isoforms also can be generated by mutagenesis. Such mutagenesis can be random or directed. For example, allelic variant isoforms can be generated that alter amino acid sequences or a potential glycosylation site to effect a change in glycosylation of an isoform, including alternate glycosylation, increased or inhibition of glycosylation at a site in an isoform. Allelic variant isoforms can be at least 90% identical in sequence to an isoform. Generally, an allelic variant isoform from the same species is at least 95%, 96%, 97%, 98%, 99% identical to an isoform, typically an allelic variant is 98%, 99%, 99.5% identical to an isoform.

For example, RAGE isoforms, including RAGE isoforms provided herein, can include allelic variation in the RAGE polypeptide. Exemplary allelic variants of RAGE are set forth in Table 3. Exemplary allelic variants of a cognate RAGE nucleotide or amino acid sequence are denoted in SEQ ID NOS: 3 and 4. Thus, a RAGE isoform can include one or more amino acid differences present in an allelic variant of a cognate RAGE. For example, a RAGE isoform can have any one or more allelic variations corresponding to those denoted in SEQ ID NO: 3 or 4. RAGE isoforms also include species variants of a cognate RAGE.

TABLE 3

| | | Nucleotide | | |
|---|---|---|---|---|
| Polymorphism | SNP NO: | change | | Amino acid change |
| NT 30 | 1800684 | 30 | A/T | none |
| NT 182 | 2555465 | 182 | T/A | none |
| NT 268 | 2070600 | 268 | G/A | AA 82 G/S |
| NT 380 | 17846807 | 380 | A/G | none |
| NT 847 | 17846800 | 847 | G/T | none |
| NT 113 | 3176931 | 1130 | G/A | AA 369 R/Q |
| AA 365 | | | | AA 365 R/S |
| AA 369 | | | | AA 369 R/G |
| AA 77 | | | | AA 77 R/C |
| AA 305 | | | | AA 305 H/Q |
| AA 307 | | | | AA 307 S/C |

F. Methods of Producing Nucleic Acid Encoding RAGE Isoforms and Methods of Producing RAGE Polypeptides

Exemplary methods for generating RAGE isoform nucleic acid molecules and polypeptides are provided herein. Such methods include molecular biology techniques known to one of skill in the art. For example, such methods include in vitro synthesis methods for nucleic acid molecules such as PCR, synthetic gene construction and in vitro ligation of isolated and/or synthesized nucleic acid fragments. RAGE isoform nucleic acid molecules also can be isolated by cloning methods, including PCR of RNA and DNA isolated from cells and screening of nucleic acid molecule libraries by hybridization and/or expression screening methods.

RAGE isoform polypeptides can be generated from RAGE isoform nucleic acid molecules using in vitro and in vivo synthesis methods. RAGE isoforms can be expressed in any organism suitable to produce the required amounts and forms of isoform needed for administration and treatment. Expression hosts include prokaryotic and eukaryotic organisms such as E. coli, yeast, plants, insect cells, mammalian cells, including human cell lines and transgenic animals. RAGE isoforms also can be isolated from cells and organisms in which they are expressed, including cells and organisms in which isoforms are produced recombinantly and those in which isoforms are synthesized without recombinant means such as genomically-encoded isoforms produced by alternative splicing events.

1. Synthetic Genes and Polypeptides

RAGE isoform nucleic acid molecules and polypeptides can be synthesized by methods known to one of skill in the art using synthetic gene synthesis. In such methods, a polypeptide sequence of a RAGE isoform is “back-translated” to generate one or more nucleic acid molecules encoding an isoform. The back-translated nucleic acid molecule is then synthesized as one or more DNA fragments such as by using automated DNA synthesis technology. The fragments are then operatively linked to form a nucleic acid molecule encoding an isoform. Nucleic acid molecules also can be joined with additional nucleic acid molecules such as vectors, regulatory sequences for regulating transcription and translation and other polypeptide-encoding nucleic acid molecules. Isoform-encoding nucleic acid molecules also can be joined with labels such as for tracking, including radiolabels, and fluorescent moieties.

The process of backtranslation uses the genetic code to obtain a nucleotide gene sequence for any polypeptide of interest, such as a RAGE isoform. The genetic code is degenerate, 64 codons specify 20 amino acids and 3 stop codons. Such degeneracy permits flexibility in nucleic acid design and generation, allowing for example restriction sites to be added to facilitate the linking of nucleic acid fragments and the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences. Back-translation is performed by selecting codons that encode a polypeptide. Such processes can be performed manually using a table of the genetic code and a polypeptide sequence. Alternatively, computer programs, including publicly available software can be used to generate back-translated nucleic acid sequences.

To synthesize a back-translated nucleic acid molecule, any method available in the art for nucleic acid synthesis can be used. For example, individual oligonucleotides corresponding to fragments of a RAGE isoform-encoding sequence of nucleotides are synthesized by standard automated methods and mixed together in an annealing or hybridization reaction. Such oligonucleotides are synthesized such annealing results in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to a RAGE isoform-encoding nucleic acid molecule, including inker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an isoform-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to isoform-encoding nucleic acid molecules. Such regions include, but are not limited to, sequences to facilitate uptake of an isoform into specific target cells, or otherwise enhance the pharmacokinetics of the synthetic gene.

RAGE isoforms also can be synthesized using automated synthetic polypeptide synthesis. Cloned and/or in silico-generated polypeptide sequences can be synthesized in fragments and then chemically linked. Alternatively, isoforms can be synthesized as a single polypeptide. Such polypeptides then can be used in the assays and treatment administrations described herein.

2. Methods of Cloning and Isolating RAGE Isoforms

RAGE isoforms can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding an isoform, include for example, polymerase chain reaction (PCR) methods. A nucleic acid containing material can be used as a starting material from which an isoform-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify an isoform. For example, primers can be designed based on expressed sequences from which an isoform is generated. Primers can be designed based on back-translation of an isoform amino acid sequence. Nucleic acid molecules generated by amplification can be sequenced and confirmed to encode an isoform.

Nucleic acid molecules encoding isoforms also can be isolated using library screening. For example, a nucleic acid library representing expressed RNA transcripts as cDNAs can be screened by hybridization with nucleic acid molecules encoding RAGE isoforms or portions thereof. For example, an intron sequence or portion thereof from a RAGE gene can be used to screen for intron retention containing molecules based on hybridization to homologous sequences. Expression library screening can be used to isolate nucleic acid molecules encoding a RAGE isoform. For example, an expression library can be screened with antibodies that recognize a specific isoform or a portion of an isoform. Antibodies can be obtained and/or prepared which specifically bind a RAGE isoform or a region or peptide contained in an isoform. Antibodies which specifically bind an isoform can be used to screen an expression library containing nucleic acid molecules encoding an isoform.

Methods of preparing and isolating antibodies, including polyclonal and monoclonal antibodies and fragments therefrom are well known in the art. Methods of preparing and isolating recombinant and synthetic antibodies also are well known in the art. For example, such antibodies can be constructed using solid phase peptide synthesis or can be produced recombinantly, using nucleotide and amino acid sequence information of the antigen binding sites of antibodies that specifically bind a candidate polypeptide. Antibodies also can be obtained by screening combinatorial libraries containing of variable heavy chains and variable light chains, or of antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an isoform polypeptide, for example, to detect the expression of a RAGE isoform in a cell, tissue or extract.

3. Expression Systems

RAGE isoforms can be produced by any method known to those of skill in the art including in vivo and in vitro methods. RAGE isoforms can be expressed in any organism suitable to produce the required amounts and forms of RAGE isoforms needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of RAGE isoforms. The choice of expression vector is influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

RAGE isoforms also can be utilized or expressed as protein fusions. For example, an isoform fusion can be generated to add additional functionality to an isoform. Examples of isoform fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Expression

Prokaryotes, especially E. coli, provide a system for producing large amounts of proteins such as RAGE isoforms. Transformation of E. coli is simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Isoforms can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach is the expression of RAGE isoforms in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are well known yeast expression hosts that can be used for production of RAGE isoforms. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast often are soluble. Co-expression with chaperonins such as Bip and protein disulfide isomerase can improved expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as RAGE isoforms. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian can be used to express RAGE isoforms. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be to express RAGE isoforms. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase often are used to facilitate selection and maintenance of transformed cells. Transformed plant cells can be maintained in culture as cells, aggregates (callus tissue) or regenerated into whole plants. Transgenic plant cells also can include algae engineered to produce RAGE isoforms (see for example, Mayfield et al. (2003) PNAS 100:438-442). Because plants have different glycosylation patterns than mammalian cells, this can influence the choice of RAGE isoforms produced in these hosts.

G. Isoform Conjugates

A variety of synthetic conjugates of RAGE isoforms are provided. A RAGE conjugate includes all or part of a RAGE polypeptide or isoform, such as a domain, intron-encoded portion, or ligand-binding portion, joined or paired with another molecule. For example, a RAGE isoform can be joined to all or part of another polypeptide or isoform, such as a domain or ligand-binding portion of another polypeptide. In some examples, a RAGE isoform containing only the extracellular ligand binding domain of a RAGE polypeptide is joined to another polypeptide that also contains only the extracellular ligand binding domain. In other instances, a RAGE isoform, such as one generated by alternative splicing as provided herein is joined to all or part of another polypeptide or molecule. The joining of the molecules can be by linkage, such as by direct or indirect linkage. In some instances, a RAGE chimeric molecule is generated where a nucleotide sequence encoding all or part of a RAGE polypeptide or isoform is fused to another nucleotide sequence encoding the same or different protein. In other instances, the conjugate is the result of covalently coupling a RAGE polypeptide or isoform to another moiety, such as for example, to a targeting agent, a fluorescent moiety, a tag, a polyethylene glycol moiety, or any other moiety known to those of skill in the art.

In one example, RAGE isoforms are provided as fusion proteins linked directly or indirectly to a nucleic acid molecule encoding another polypeptide, such as a polypeptide that promotes secretion of an isoform. In some examples, a fusion protein can result in a chimeric polypeptide. For example, a chimera can include a polypeptide in which the extracellular domain portion and C-terminal portion, such as an intron encoded portion, are from different isoforms. In another example, a fusion protein containing, for example, a multimerization domain, can result in a homodimeric or heterodimeric molecule.

Also included among synthetic forms are conjugates in which a RAGE isoform, or intron-encoded portion thereof, is linked directly or via a linker to another agent, such as a targeting agent or target agent or to any other molecule that presents a RAGE isoform or intron-encoded portion of a RAGE isoform to cell surface receptor (CSR), such as to RAGE, so that an activity of the CSR is modulated. Also provided are “peptidomimetic” isoforms in which one or more bonds in the peptide backbone is (are) replaced by a bioisotere or other bond such that the resulting polypeptide peptidomimetic has improved properties, such as resistance to proteases, compared to the unmodified form.

RAGE isoform conjugates can be designed and produced with one or more modified properties. These properties include, but are not limited to, increased production including increased secretion or expression. For example, a RAGE isoform can be modified to exhibit improved secretion compared to an unmodified RAGE isoform. Other properties include increased protein stability, such as an increased protein half-life, increased thermal tolerance and/or resistance to one or more proteases. For example, a RAGE isoform can be modified to increase protein stability in vitro and/or in vivo. In vivo stability can include protein stability under particular administration conditions such as stability in blood, saliva, and/or digestive fluids.

RAGE isoforms also can be modified to exhibit modified properties without producing a conjugated polypeptide using any methods known in the art for modification of proteins. Such methods can include site-directed and random mutagenesis. Non-natural amino acids and/or non-natural covalent bonds between amino acids of the polypeptide can be introduced into a RAGE isoform to increase protein stability. In such modified RAGE isoforms, the biological function of the isoform can remain unchanged compared to the unmodified isoform. In some examples, a modified RAGE isoform also can be provided as a conjugate such as a fusion protein, chimeric protein, or other conjugate provided herein. Assays such as the assays for biological function provided herein and known in the art can be used to assess the biological function of a modified RAGE isoform.

Linkage of a synthetic RAGE isoform as a fusion protein or synthetic conjugate can be direct or indirect. In some examples, linkage can be facilitated by nucleic acid linkers such as restriction enzyme linkers, or other peptide linkers that promote the folding or stability of an encoded polypeptide. Linkage of a polypeptide conjugate also can be by chemical linkage or facilitated by heterobifunctional linkers, such as any known in the art or provided herein. Exemplary peptide linkers and heterobifunctional cross-linking reagents are provided below. For example, Exemplary linkers include, but are not limited to, (Gly4Ser)n, (Ser4Gly)n and (AlaAlaProAla)n (see, SEQ ID NO. 319) in which n is 1 to 6, such as 1, 2, 3 or 4, such as:

| | |
|---|---|
| (1) | Gly4Ser with NcoI ends SEQ ID NO. 320 |
| | CCATGGGCGG CGGCGGCTCT GCCATGG |
| (2) | (Gly4Ser)2 with NcoI ends SEQ ID NO. 321 |
| | CCATGGGCGG CGGCGGCTCT GGCGGCGGCG GCTCTGCCAT GG |
| (3) | (Ser4Gly)4 with NcoI ends SEQ ID NO. 322 |
| | CCATGGCCTC GTCGTCGTCG GGCTCGTCGT CGTCGGGCTC |
| | GTCGTCGTCG GGCTCGTCGT CGTCGGGCGC CATGG |
| (4) | (Ser4Gly)2 with NcoI ends SEQ ID NO. 323 |
| | CCATGGCCTC GTCGTCGTCG GGCTCGTCGT CGTCGGGCGC |
| | CATGG |
| (5) | (AlaAlaProAla)n, where n is 1 to 4, such as 2 |
| | or 3 (see, SEQ ID NO.:324) |

Numerous heterobifunctional cross-linking reagents that are used to form covalent bonds between amino groups and thiol groups and to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad. Sci. 84:308-312; Walden et al. (1986) J. Mol. Cell. Immunol. 2:191-197; Carlsson et al. (1978) Biochem. J. 173: 723-737; Mahan et al. 91987) Anal. Biochem. 162:163-170; Wawryznaczak et al. (1992) Br. J. Cancer 66:361-366; Fattom et al. (1992) Infection & Immun. 60:584-589). These reagents may be used to form covalent bonds between the N-terminal portion and C-terminus intron-encoded portion or between each of those portions and a linker. These reagents include, but are not limited to: N-succinimidyl-3-(2-pyridyldithio)propionate (SPDP; disulfide linker); sulfosuccinimidyl 6-[3-(2-pyridyldithio)propion [symbol] amido]hexanoate (sulfo-LC-SPDP); succinimidyloxycarbonyl-α-methyl benzyl thiosulfate (SMBT, hindered disulfate linker); succinimidyl 6-[3-(2-pyridyldithio)propionami [symbol] do] [symbol] hexanoate (LC-SPDP); sulfosuccinimidyl 4-(N-maleimidomethyl)cyclohexane-1-carboxylate (sulfo-SMCC); succinimi [symbol] dyl 3-(2-pyridyldithio)butyrate (SPDB; hindered disulfide bond linker); sulfosuccinimidyl 2-(7-azido-4-methylcoumarin-3-acetamide)ethyl-1,3′-dithiopropionate (SAED); sulfosuccinimidyl 7-azido-4-methylcoumarin-3-acetate (SAMCA); sulfosuccinimidyl-6-[alpha-methyl-alpha-(2-pyridyldithio)toluamido]-hexanoate (sulfo-LC-SMPT); 1,4-di-[3′-(2′-pyridyldithio)propion-amido]butane (DPDPB); 4-succinimidyloxycarbonyl-α-methyl-α-(2-pyridylthio)toluene (SMPT, hindered disulfate linker); sulfosuccinimidyl-6-[α-methyl-α-(2-pyrimiyldi-thio)toluamido]hexanoate (sulfo-LC-SMPT); m-maleimidobenzoyl-N-hydroxy-succinimide ester (MBS); m-maleimidobenzoyl-N-hydroxysulfo-succinimide ester (sulfo-MBS); N-succinimidyl(4-iodoacetyl)aminobenzoate (SIAB; thioether linker); sulfosuccinimidyl-(4-iodoacetyl)amino benzoate (sulfo-SIAB); succinimidyl-4-(p-maleimi-dophenyl)butyrate (SMPB); sulfosuccinimidyl-4-(p-maleimido-phenyl)buty-rate (sulfo-SMPB); azidobenzoyl hydrazide (ABH). These linkers, for example, can be used in combination with peptide linkers, such as those that increase flexibility or solubility or that provide for or eliminate steric hindrance. Any other linkers known to those of skill in the art for linking a polypeptide molecule to another molecule can be employed. General properties are such that the resulting molecule is biocompatible (for administration to animals, including humans) and such that the resulting molecule modulates the activity of a cell surface molecule, such as a RAGE receptor, or other cell surface molecule or receptor.

Pharmaceutical compositions can be prepared that contain RAGE isoform conjugates and treatment effected by administering a therapeutically effective amount of a conjugate, for example, in a physiologically acceptable excipient. RAGE isoform conjugates also can be used in in vivo therapy methods such as by delivering a vector containing a nucleic acid encoding a RAGE isoform conjugate as a fusion protein.

1. Isoform Fusions

Exemplary of RAGE conjugates are RAGE isoform fusions, which include linkage of a nucleic acid sequence of RAGE with another nucleic acid sequence. Nucleic acid molecules that can be joined to a RAGE isoform, include but are not limited to, promoter sequences designed to facilitate intracellular protein expression, secretion sequences designed to facilitate protein secretion, regulatory sequences for regulating transcription and translation, molecules that regulate the serum stability of an encoded polypeptide such as portions of CD45 or an Fc portion of an immunoglobulin, and other polypeptide-encoding nucleic acid molecules such as those encoding a targeted agent or targeting agent, or those encoding all or part of another ligand or cell surface receptor intron fusion protein. The fusion sequence can be a component of an expression vector, or it can be part of an isoform nucleic acid sequence that is inserted into an expression vector. The fusion can result in a chimeric protein encoded by two or more genes, or the fusion can result in a protein sequence encoding only an RAGE isoform polypeptide, such as if the fused sequence is a signal sequence that is cleaved off following secretion of the polypeptide into the secretory pathway. In one example, a nucleic acid fused to all or part of a RAGE isoform can include any nucleic acid sequence that improves the production of an isoform such as a promoter sequence, epitope or fusion tag, or a secretion signal. In another example, a RAGE isoform fusion can include fusion with a targeted agent or targeting agent to produce a RAGE isoform conjugate such as described below. Additionally, a nucleic acid encoding all or part of a RAGE isoform can be joined to a nucleic acid encoding another ligand or cell surface receptor intron fusion isoform, or intron portion thereof, thereby generating a chimeric intron fusion protein. Exemplary RAGE chimeras are described below.

Encoded RAGE isoform fusion proteins can contain additional amino acids which do not adversely affect the activity of a purified isoform protein. For example, additional amino acids can be included in the fusion protein as a linker sequence which separate the encoded isoform protein from the encoded fusion sequence in order to provide, for example, a favored steric configuration in the fusion protein. The number of such additional amino acids which can serve as separators can vary, and generally do not exceed 60 amino acids. In another example, a fusion protein can contain amino acid residues encoded by a restriction enzyme linker sequence. In an additional example, an isoform fusion protein can contain selective cleavage sites at the junction or junctions between the fusion of a RAGE isoform with another molecule. For example, such selective cleavage sites may comprise one or more amino acid residues which provide a site susceptible to selective enzymatic, proteolytic, chemical, or other cleavage. In one example, the additional amino acids can be a recognition site for cleavage by a site-specific protease. The fusion protein can be further processed to cleave the fused polypeptide therefrom; for example, if the isoform protein is fused to an epitope tag but is required without additional amino acids such as for therapeutic purposes.

a. RAGE Isoform Fusions for Improved Production of RAGE Isoform Polypeptides

Provided herein are nucleic acid sequences encoding RAGE fusion polypeptides for the improved production of a RAGE isoform. A nucleic acid of a RAGE isoform, such as set forth in any one of SEQ ID NOS: 5-9 can be fused to a homologous or heterologous precursor sequence that substitutes for and/or provides for a functional secretory sequence. In one example, an isoform, such as an intron fusion protein isoform, containing a native endogenous precursor signal sequence of a cognate RAGE can have its precursor sequence replaced with a heterologous or homologous precursor sequence, such as a precursor sequence of tissue plasminogen activator or any other signal sequence known to one of skill in the art, to improve the secretion and production of a RAGE isoform polypeptide. The precursor sequence is most effectively utilized by locating it at the N-terminus of a recombinant protein to be secreted from the host cell. A nucleic acid precursor sequence can be operatively joined to a nucleic acid containing the coding region of a RAGE isoform in such a manner that the precursor sequence coding region is upstream of (that is, 5′ of) and in the same reading frame with the isoform coding region to provide an isoform fusion. The isoform fusion can be expressed in a host cell to provide a fusion polypeptide comprising the precursor sequence joined, at its carboxy terminus, to a RAGE isoform at its amino terminus. The fusion polypeptide can be secreted from a host cell. Typically, a precursor sequence is cleaved from the fusion polypeptide during the secretion process, resulting in the accumulation of a secreted isoform in the external cellular environment or, in some cases, in the periplasmic space.

Optionally a RAGE isoform, including an intron fusion protein, fusion nucleic acid also can include operative linkage with another nucleic acid sequence or sequences, such as a sequence that encodes a fusion tag, that promotes the purification and/or detection of an isoform polypeptide. Non-limiting examples of fusion tags include a myc tag, Poly-His tag, GST tag, Flag tag, fluorescent or luminescent moiety such as GFP or luciferase, or any other epitope or fusion tag known to one of skill in the art. In other embodiments, a nucleic acid sequence of a RAGE isoform can contain an endogenous signal sequence and can include fusion with a nucleic acid sequence encoding a fusion tag or tags. Many precursor sequences, including signal sequences and prosequences, and/or fusion tag sequences have been identified and are known in the art, and are contemplated to be used in conjunction with an isoform nucleic acid molecule. A precursor sequence may be homologous or heterologous to an isoform gene or cDNA, or a precursor sequence can be chemically synthesized. In most cases, the secretion of an isoform polypeptide from a host cell via the presence of a signal peptide and/or propeptide will result in the removal of the signal peptide or propeptide from the secreted intron fusion protein polypeptide.

i. Tissue Plasminogen Activator

Tissue plasminogen activator (tPA) is a serine protease that regulates hemostasis by converting the zymogen plasminogen to its active form, plasmin. Like other serine proteases, tPA is synthesized and secreted as an inactive zymogen that is activated by proteolytic processing. Specifically, the mature partially active single chain zymogen form of tPA can be further processed into a two-chain fully active form by cleavage after Arg-310 of SEQ ID NO:329 catalyzed by plasmin, tissue kallikrein or factor Xa. tPA is secreted into the blood by endothelial cells in areas immediately surrounding blood clots, which are areas rich in fibrin. tPA regulates fibrinolysis due to its high catalytic activity for the conversion of plasminogen to plasmin, a regulator of fibrin clots. Plasmin also is a serine protease that becomes converted into a catalytically active, two-chain form upon cleavage of its zymogen form by tPA. Plasmin functions to degrade the fibrin network of blood clots by cutting the fibrin mesh at various places, leading to the production of circulating fragments that are cleared by other proteinases or by the kidney and liver.

The precursor polypeptide of tPA includes a pre-sequence and pro-sequence encoded by residues 1-35 of a full-length tPA sequence set forth in SEQ ID NO:329 and exemplified in SEQ ID NO:327. The precursor sequence of tPA contains a signal sequence including amino acids 1-23 and also contains two pro-sequences including amino acids 24-32 and 33-35 of an exemplary tPA sequences set forth in SEQ ID NO: 327 or 329. The signal sequence of tPA is cleaved co-translationally in the ER and a pro-sequence is removed in the Golgi apparatus by cleavage at a furin processing site following the sequence RFRR occurring at amino acids 29-32 of the exemplary sequences set forth in SEQ ID NO:327 or 329. Furin cleavage of a tPA pro-sequence retains a three amino acid pro-sequence and exopeptidase cleavage site GAR, set forth as amino acids 33-35 of an exemplary tPA sequence set forth in SEQ ID NO: 327 or 329, within a mature polypeptide tPA sequence. The cleavage of the retained pro-sequence site is mediated by a plasmin-like extracellular protease to obtain a mature tPA polypeptide beginning at Ser36 set forth in SEQ ID NO:327 or 329. Inclusion of a protease inhibitor, such as for example aprotinin, in the culture medium can prevent exopeptidases cleavage and thereby retain a GAR pro-sequence in the mature polypeptide of tPA (Berg et al., (1991) Biochem Biophys Res Comm, 179:1289).

Typically, tPA is secreted by the constitutive secretory pathway, although in some cells tPA is secreted in a regulated manner. For example, in endothelial cells regulated secretion of tPA is induced following endothelial cell activation, for example, by histamine, platelet-activating factor or purine nucleotides, and requires intraendothelial Ca2+ and cAMP signaling (Knop et al., (2002) Biochem Biophys Acta 1600:162). In other cells, such as for example neural cells, specific stimuli that can induce secretion of tPA include exercise, mental stress, electroconvulsive therapy, and surgery (Parmer et al., (1997) J Biol Chem 272:1976). The mechanism mediating the regulated secretion of tPA requires signals on the tPA polypeptide itself, whereas the signal sequence of tPA efficiently mediates constitutive secretion of tPA since a GFP molecule operatively linked only to the signal sequence of tPA is constitutively secreted in the absence of carbachol stimulation (Lochner et al., (1998) Mol Biol Cell, 9:2463). In the absence of a tPA signal sequence, a tPA/GFP hybrid protein is not secreted from cells.

An exemplary tPA precursor sequence including a pre/propeptide sequence of tPA is set forth in SEQ ID NO: 327, and is encoded by a nucleic acid sequence set forth in SEQ ID NO:326. The signal sequence of tPA includes amino acids 1-23 of SEQ ID NO:329 and the pro-sequence includes amino acids 24-35 of SEQ ID NO:329 whereby a furin-cleaved pro-sequence includes amino acids 24-32 and a plasmin-like exoprotease-cleaved pro-sequence includes amino acids 33-35. Allelic variants of a tPA pre/prosequence are also provided herein, such as those set forth in SEQ ID NOS:330 or 331. Further, isoform protein fusion of a pre/prosequence of mammalian and non-mammalian origin of tPA are contemplated and exemplary sequences are set forth in SEQ ID NOS:332-339.

ii. tPA-RAGE Isoform Fusions

Provided herein are nucleic acid sequences encoding tPA-RAGE isoform polypeptides, for the improved production of a RAGE intron fusion protein isoform. Such nucleic acid sequences contain all or part of a pre/prosequence of tPA and optionally a c-myc fusion tag for the improved production of a RAGE intron fusion protein polypeptide. Nucleic acid sequences encoding RAGE isoforms, including intron fusion protein isoforms of RAGE, or allelic variants thereof, such as any one of SEQ ID NOS: 5-9, encoding amino acids set forth in SEQ ID NOS:10-14, operatively linked to a tPA pre/prosequence are provided. A tPA pre/prosequence can include a tPA pre/prosequence set forth as SEQ ID NO:326 encoding amino acids set forth as 1-35 in SEQ ID NO:327. In some examples, a tPA pre/pro sequence can replace the endogenous precursor signal sequence of RAGE, such as for example amino acids corresponding to amino acids 1-22 of a cognate RAGE set forth in SEQ ID NO:2, and/or provide for an optimal precursor sequence for the secretion of an intron fusion protein polypeptide.

In other embodiments, a RAGE isoform or allelic variants thereof, set forth in any one of SEQ ID NOS: 5-9, encoding amino acids set forth in SEQ ID NOS:10-14, can be operatively linked to part of a tPA pre/prosequence including the nucleic acid sequence up to the furin cleavage site of a pre/prosequence of tPA (encoded amino acids 1-32 of an exemplary tPA pre-prosequence set forth in SEQ ID NO:326), thereby excluding nucleic acids encoding amino acids GAR (encoded amino acids 33-35 of an exemplary tPA pre-prosequence set forth in SEQ ID NO:327). Additionally, a nucleic acid sequence of a RAGE isoform or allelic variants thereof, such as set forth in any one of SEQ ID NOS: 5-9, encoding amino acids set forth in SEQ ID NOS:10-14, can include operative linkage with allelic variants of all or part of a tPA pre/prosequence, such as set forth in SEQ ID NOS: 330 or 331 or can include operative linkage with all or part of other tPA pre/prosequences of mammalian and non-mammalian origin, such as set forth in any one of SEQ ID NOS:332-339. RAGE intron fusion protein-tPA pre/pro fusion sequences provided herein can exhibit enhanced cellular expression and secretion of a RAGE isoform polypeptide for improved production.

In another embodiment, a nucleic acid sequence encoding a RAGE isoform or allelic variant thereof, such as any one of SEQ ID NOS: 5-9, encoding amino acids set forth in SEQ ID NOS:10-14, can include operative linkage with a presequence (signal sequence) only of a tPA pre/prosequence such as an exemplary signal sequence encoding amino acids 1-23 of an exemplary tPA pre/prosequence set forth as SEQ ID NO:327. RAGE intron fusion protein-tPA presequence fusions provided herein can exhibit enhanced cellular expression and secretion of a RAGE isoform polypeptide for improved production.

In an additional embodiment, a nucleic acid sequence encoding a RAGE isoform or allelic variant thereof, such as any one of SEQ ID NOS: 5-9, encoding amino acids set forth in SEQ ID NOS:10-14, that contains an endogenous signal sequence of a cognate RAGE can include a fusion with a tPA prosequence where insertion of a tPA prosequence is between the RAGE isoform endogenous signal sequence and the RAGE isoform coding sequence. In one example, a tPA prosequence includes a nucleic acid sequence encoding amino acids 24-32 of an exemplary tPA pre/prosequence set forth as SEQ ID NO:326. In another example, a tPA pro-sequence includes a nucleic acid sequence encoding amino acids 33-35 of an exemplary tPA pre/prosequence set forth as SEQ ID NO:326. In an additional example, a tPA prosequence includes a nucleic acid sequence encoding amino acids 24-35 of an exemplary tPA pre/prosequence set forth as SEQ ID NO:326. Other tPA prosequences can include amino acids 24-32, 33-35, or 24-35 of allelic variants of tPA pre/prosequences such as set forth in SEQ ID NOS:330 or 331. RAGE intron fusion protein-tPA prosequence fusions provided herein can exhibit enhanced cellular expression and secretion of a RAGE isoform polypeptide for improved production.

Additionally, a RAGE isoform, RAGE intron fusion protein-tPA pre/prosequence fusion, RAGE intron fusion protein-tPA presequence fusion, and/or a RAGE intron fusion protein-tPA prosequence fusion for the improved secretion of an intron fusion protein polypeptide can optionally also include one, two, three, or more fusion tags that facilitate the purification and/or detection of a RAGE isoform polypeptide. Generally, a coding sequence for a specific tag can be spliced in frame on the amino or carboxy ends, with or without a linker region, with a coding sequence of a nucleic acid molecule encoding a RAGE isoform polypeptide. When fusion is on an amino terminus of a sequence, a fusion tag can be placed between an endogenous or heterologous precursor sequence. In one embodiment a fusion tag, such as a c-myc tag, 8×His tag, or any other fusion tag known to one of skill in the art, can be placed between a RAGE isoform endogenous signal sequence and a RAGE coding sequence. In another embodiment, a fusion tag can be placed between a heterologous precursor sequence, such as a tPA pre/prosequence, presequence, or prosequence set forth in SEQ ID NO:326, and a RAGE isoform coding sequence. In other embodiments, a fusion tag can be placed directly on the carboxy terminus of a nucleic acid encoding a RAGE isoform fusion polypeptide sequence. In some instances, a RAGE isoform fusion can contain a linker between an endogenous or heterologous precursor sequence and a fusion tag. RAGE isoform fusions containing one or more fusion tag(s) provided herein, including RAGE intron fusion protein-tPA fusions, can facilitate easier detection and/or purification of a RAGE isoform polypeptide for improved production.

For example, for the exemplary RAGE isoform provided herein as SEQ ID NO: 13 amino acids 1-23 of the RAGE isoform, including the endogenous signal sequence containing amino acids 1-22, can be replaced by a tPA pre/prosequence, such as for example, the exemplary tPA pre/prosequence set forth as SEQ ID NO: 327 and encoded by a tPA pre/prosequence set forth as SEQ ID NO:326. For example, the nucleic acid sequence of an exemplary tPA-RAGE intron fusion protein fusion set forth in SEQ ID NO:340, encoding a polypeptide set forth in SEQ ID NO: 341, can include the nucleic acid sequence encoding amino acids 23-266 of the RAGE isoform set forth in SEQ ID NO: 13 operatively linked at the 5′ end to a sequence containing a tPA pre/prosequence (nucleotides 1-105 of SEQ ID NO:340) followed by a sequence containing an XhoI restriction enzyme linker site (nucleotides 136-141 of SEQ ID NO:340). Optionally, a sequence of an exemplary tPA-RAGE intron fusion protein fusion set forth in SEQ ID NO:340, encoding a polypeptide set forth in SEQ ID NO:341, also can include a myc epitope tag set forth as nucleotides 106-135 operatively fused between the tPA pre/prosequence and the Xho I linker site.

b. Chimeric and Synthetic RAGE Isoform Polypeptides Including Homo- and Heteromultimeric Polypeptides

Also provided are chimeric RAGE isoform polypeptides. A chimeric RAGE isoform is a protein encoded by all or part of two or more genes resulting in a polypeptide containing all or part of an encoded RAGE sequence operatively linked to another polypeptide. Generally, such chimeric polypeptides are oligomeric (multimeric) molecules. Generally, the oligomers are dimers or trimers. Dimeric and trimeric forms of RAGE isoforms can exhibit enhanced activity compared to the monomeric form and/or can exhibit one or more additional activities as compared to a RAGE isoform. In some instances multimers are formed between the same RAGE isoform, different RAGE isoforms, or a RAGE isoform and another polypeptide isoform. Generally such isoforms are soluble forms of a cognate receptor or ligand and thereby lack a transmembrane domain. In most instances, such isoforms contain all or a sufficient portion of the extracellular domain such that they retain their ability to bind to ligand. In some examples, the isoforms are intron fusion proteins. Separate encoded polypeptide chains can be joined by multimerization, such as for example, by interchain disulfide bonds formed between cysteine residues to form oligomers. Alternatively, the multimers can be expressed as fusion proteins, with or without a spacer amino acids between a RAGE isoform and another isoform moiety, using recombinant DNA techniques. In some examples, two, three, or more encoded isoform polypeptides, including a RAGE isoform polypeptide, can be joined via a polypeptide linker.

Generally, such heteromultimeric polypeptides will retain the ability to bind their respective ligand. For example, a heteromultimeric polypeptide including a RAGE isoform retains its ability to bind AGEs, for example, and its ability to bind a ligand of the partner multimeric polypeptide. Consequently, such heteromultimeric polypeptides can serve as antagonist to one or more than one cognate receptor.

In one example, a chimeric RAGE isoform contains all or part of a RAGE isoform, including an intron from a RAGE intron fusion polypeptide, operatively linked at the N-terminus to another polypeptide or other molecule such that the resulting molecule modulates the activity of a cell surface molecule, particularly a RAGE receptor or RTK receptor, including any involved in pathways that participate in the inflammatory response, angiogenesis, neovascularization and/or cell proliferation. Included among these synthetic “polypeptides” are chimeric intron fusion polypeptides in which all or part of a RAGE isoform is linked to all or part of an intron fusion protein, such as for example any one of the sequences of intron fusion proteins disclosed in U.S. patent application Ser. No. 10/846,113 or 11/129,740, incorporated by reference in their entirety, or set forth in any of the sequences and encoded amino acids set forth in any one of SEQ ID NOS: 28, 30, 32-50, 52, 54, 56, 58, 60, 62, 64, 66, 68, 70, 72, 74, 76, 78, 80, 82, 84, 86, 88, 90, 92, 105, 107, 109, 111, 113, 115, 17, 119, 121, 123, 125, 127, 129, 131, 133, 135, 137, 139, 141, 143, 145, 147, 149, 151, 153, 155, 157, 159, 161, 163, 306-318, and 344-348. For example, a chimeric RAGE includes a polypeptide in which all or part of the N-terminus from the extracellular domain of a RAGE isoform is linked to the intron of an intron fusion protein, such as intron 8 of a herstatin (see, e.g., SEQ ID Nos. 278-291, and encoded amino acids set forth in SEQ ID NOS: 253, 266-277, 342). Exemplary herstatins, or intron 8 portions thereof, are set forth in SEQ ID NOS: 252-291, 342, and 343). Table 4 below identifies the variations in the intron 8-encoded portion of a herstatin compared to a prominent intron 8 (SEQ ID NO:253), included at amino acids 341-419 of the prominent herstatin molecule set forth as SEQ ID NO:252. The sequence identifiers (SEQ ID NOS) for exemplary intron 8 and herstatin molecules, including variants of an intron 8 or herstatin, are in parentheses. Other herstatin variants include allelic variants, particularly those with variation in the extracellular domain portion.

TABLE 4
Herstatin Variants

| Intron 8 Variant | | Herstatin variant |
|---|---|---|
| Nucleotide | Amino Acid | Amino Acid |
| Prominent (278) | Prominent (253) | Prominent (252) |
| nt 4 = T (279) | aa 2 = Thr or Ser (266) | aa 342 = Thr or Ser (254) |
| nt 14 = C (280) | aa 5 = Leu or Pro (267) | aa 345 = Leu or Pro (255) |
| nt 17 = T (281) | aa 6 = Pro or Leu (268) | aa 346 = Pro or Leu (256) |
| nt 47 = A (282) | aa 16 = Leu or Gln (269) | aa 356 = Leu or Gln (257) |
| nt 52 = C (283) | aa 18 = Met or Leu (270) | aa 358 = Met or Leu (258) |
| nt 62 = C, T, A | aa 21 = Gly, Asp, Ala, | aa 361 = Gly, Asp, Ala, |
| (284) | Val (271) | Val (259) |
| nt 92 = T (291) | aa 31 = Arg or Ile (277) | aa 371 = Arg or Ile (265) |
| nt 106 = A (285) | aa 36 = Leu or Ile (272) | aa 376 = Leu or Ile (260) |
| nt 161 = G (286) | aa 54 = Pro or Arg (273) | aa 394 = Pro or Arg (261) |
| nt 191 = T (287) | aa 64 = Pro or Leu (274) | aa 404 = Pro or Leu (262) |
| nt 217 = C or A | aa 73 = Asp, His, or Asn | aa 413 = Asp, His or Asn |
| (288) | (275) | (263) |
| nt 49 = T (290) | aa 17 = Arg or Cys (276) | aa 357 = Arg or Cys (264) |
| nt 17 = T and nt | aa 6 = Leu and aa 73 = | aa 346 = Leu and aa 413 = |
| 217 = C or A | His or Asn (342) | His or Asn (343) |
| (289) | | |

Chimeric and synthetic RAGE isoform fusions also include fusion of nucleic acid encoding a RAGE isoform provided herein, with a nucleic acid encoding another RAGE isoform provided herein or known to skill in the art. For example, RAGE isoforms provided herein can be linked directly or indirectly to all or part of a RAGE isoform, such as for example, a RAGE isoform sequence encoding amino acids set forth in any one of SEQ ID NOS: 292-305.

The N- or C-terminus portion of a RAGE isoform can be linked directly to the N- or C-terminus (intron-encoded portion) of the synthetic intron fusion protein or to another polypeptide, or can be linked via a linker, such as a polypeptide linker. Linkage can be effected by recombinant expression of a fusion protein where there is no linker or where the linker is a polypeptide. For example, linkage can be effected by recombinant expression of a fusion protein where all or part of a nucleic acid encoding a RAGE isoform is operatively linked at the 5′ end to all or part of a nucleic acid encoding another intron fusion protein. Linkage can be in the presence of an encoded peptide linker such as any linker described herein or known in the art, or in the presence of a restriction enzyme linker.

In some instances, chemical synthesis also can be employed. For example, when the linker is not a polypeptide, linkage can be effected chemically. In such instances, a RAGE isoform encoded polypeptide also can be linked or conjugated to all or part of another polypeptide by chemical linkage such as by using a heterobifunctional cross-linking reagent or any other linkage that can be effected chemically such as is described above.

Any suitable linker can be selected so long as the resulting molecule interacts with a CSR and modulates, typically inhibits, its activity. Linkers can be selected to add a desirable property, such as to increase serum stability, solubility and/or intracellular concentration and to reduce steric hindrance caused by close proximity where one or more linkers is (are) inserted between the N-terminal portion and intron-encoded portion. The resulting molecule is designed or selected to retain the ability to modulate the activity of a CSR, particularly RTKs, including any involved in pathways that are involved in inflammatory responses, neovascularization, angiogenesis and cell proliferation.

Linkers include chemical linkers and peptide linkers, such as peptides that increase flexibility or solubility of the linked moieties. For example linkers can be inserted using heterobifunctional reagents, such as those described above, or, can be linked by linking DNA encoding polypeptide linker to the DNA encoding the N-terminal (and/or C-terminal portion) and expressing the resulting chimera. In addition, where no linker is present the N-terminus can be linked directly to the intron encoded portion. In some embodiments, the N-terminus portion can be replaced by a non-peptidic moiety that provides sufficient steric hindrance and bulk to permit the intron-encoded portion to interact with and modulate the activity of a receptor. As noted above, the N-terminus also can be selected to target the intron-encoded portion to selected CSRs or a selected CSR.

In some instances, heterodimers can be prepared by expression of chimeric molecules utilizing flexible linker loops. For example, a DNA construct encoding a chimeric protein is designed such that it expresses two isoforms, such as for example a RAGE isoform and another isoform polypeptide, fused together in tandem (“head to head”) by a flexible loop. This loop can be entirely artificial (e.g., polyglycine repeats interrupted by serine or threonine at certain intervals), or “borrowed” from naturally occurring proteins (e.g., the hinge region of hIgG). Molecules can be engineered in which the order of the isoforms fused is switched (e.g., RAGE isoform/loop/X isoform or X isoform/loop/RAGE isoform, where X is another polypeptide isoform that can be the same or different from the RAGE isoform). In addition, molecules can be engineered in which the length and composition of the loop is varied, to allow for selection of molecules with desired characteristics.

Also provided are homo- or heteromultimeric RAGE isoform polypeptides generated from separate chimeric fusion polypeptides. Such polypeptides include chimeric fusions, such as for example, fusion (directly or indirectly) of a nucleic acid encoding a RAGE isoform with a nucleic acid encoding a multimerization domain and a second polypeptide chimeric fusion of a nucleic acid encoding the same or different polypeptide isoform with a nucleic acid encoding a multimerization domain. Typically, the multimerization domain provides for the formation of a stable protein-protein interaction between a first polypeptide chimeric fusion and a second polypeptide chimeric fusion. The first and second chimeric fusion can be the same or different. A heteromultimer isoform fusion polypeptide includes two or more chimeric isoform polypeptides, or an active portion thereof, including a RAGE isoform such as provided herein, and/or another polypeptide isoform. Generally, a homo- or heteromultimer provided herein contains as a multimerization partner at least one RAGE isoform, such as for example any provided herein and set forth in SEQ ID NOS: 5-9, encoding amino acids set forth in SEQ ID NO:10-14. For example, a homomultimeric RAGE isoform polypeptide can result from the multimerization of the same chimeric RAGE isoform fusion. In another example, a heteromultimeric RAGE isoform polypeptide can result from the multimerization of a chimeric RAGE isoform fusion with another chimeric fusion polypeptide. Generally, such other polypeptide fusion contains all or part of the extracellular domain (ECD) of a cell surface receptor (CSR), such as for example, a receptor tyrosine kinase (RTK). Exemplary polypeptides for the generation of heteromultimeric polypeptides with a RAGE isoform provided herein, include any CSR or ligand isoform such as for example any described in U.S. patent application Ser. No. 10/846,113 or 11/129,740, incorporated by reference in their entirety, or set forth in any of the sequences and encoded amino acids set forth in any one of SEQ ID NOS: 28, 30, 32-50, 52, 54, 56, 58, 60, 62, 64, 66, 68, 70, 72, 74, 76, 78, 80, 82, 84, 86, 88, 90, 92, 105, 107, 109, 111, 113, 115, 17, 119, 121, 123, 125, 127, 129, 131, 133, 135, 137, 139, 141, 143, 145, 147, 149, 151, 153, 155, 157, 159, 161, 163, 306-318, and 344-348. Other exemplary polypeptides include any RAGE isoform known in the art, such as for example, any having an encoded amino acid sequence set forth in any one of SEQ ID NOS: 292-305.

Generally, a multimerization domain includes any capable of forming a stable protein-protein interaction. The multimerization domains can interact via an immunoglobulin sequence, leucine zipper, a hydrophobic region, a hydrophilic region, or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. patent application Ser. No. 08/399,106. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or typtophan). Compensatory cavities of identical or similar size to the protuberances are optionally created on the interface of the second polypeptide by replacing large amino acid side chains with smaller ones (e.g., alanine or threonine). In addition, multimerization domains include those comprising a free thiol moiety capable of reacting to form an intermolecular disulfide bond with a multimerization domain of an additional amino acid sequence. For example, a multimerization domain can include a portion of an immunoglobulin molecule, such as from IgG1, IgG2, IgG3, IgG4, IgA, IgD, IgM, and IgE. Generally, such portion is an immunoglobulin constant region (Fc). In another example, a multimerization domain is a polyethylene glycol (PEG) moiety others.

In one example, a RAGE isoform, and/or other polypeptide isoform, is engineered using leucine zippers. The leucine zipper domains of the human transcription factors c-jun and c-fos have been shown to form stable heterodimers (see e.g., Busch and Sassone-Corsi (1990) Trends Genetics, 6: 36-40; Gentz et al., (1989) Science, 243: 1695-1699) with a 1:1 soichiometry. Although jun-jun homodimers also have been shown to form, they are about 1000-fold less stable than jun-fos heterodimers. Thus, typically heterodimers are generated using a jun-fos combination, for example heterodimers of a RAGE isoform dimerized to another polypeptide through leucine zipper interactions. Generally, the leucine zipper domain of either c-jun or c-fos are fused in frame at the C-terminus of the soluble or extracellular domains of polypeptide isoforms, such as RAGE isoforms, by genetically engineering chimeric genes. Exemplary encoded amino acid sequences of a c-jun and c-fos leucine zipper are set forth in SEQ ID NOS: 364 and 365, respectively. In some instances, sequence of a leucine zipper can be modified, such as by the addition of a cysteine residue to allow formation of disulfide bonds, or the addition of a tyrosine residue at the C-terminus to facilitate measurement of peptide concentration. Exemplary sequences of encoded amino acid sequences of a modified c-jun and c-fos leucine zipper are set forth in SEQ ID NOS: 366 and 367, respectively. In addition, the fusions can be direct or can employ a flexible linker domain, such as for example a hinge region of IgG, or polypeptide linkers of small amino acids such as glycine, serine, threonine, or alanine at various lengths and combinations. In some instances, separation of a leucine zipper from the C-terminus of an encoded polypeptide isoform can be effected by fusion with a sequence encoding a protease cleavage site, such as for example, a thrombin cleavage site. Additionally, the chimeric proteins can be tagged, such as for example, by a 6×His tag, to allow rapid purification by metal chelate chromatography and/or by epitopes to which antibodies are available, such as for example a myc tag, to allow for detection on western blots, immunoprecipitation, or activity depletion/blocking bioassays.

In another example, an Fc-domain can be employed as a multimerization domain. For example, the Fc domain of human IgG1 can be used (see e.g., Aruffo et al., (1991) Cell, 67:35-44). In this instance, formation of heterodimers must be biochemically achieved, as chimeric molecules carrying the Fc-domain will be expressed as disulfide-linked homodimers as well. Thus, homodimers can be reduced under conditions that favor the disruption of inter-chain disulfides, but do not effect intra-chain disulfides. Typically, chimeric monomers with different extracellular portions are mixed in equimolar amounts and oxidized to form a mixture of homo- and heterodimers. The components of this mixture are separated by chromatographic techniques. Alternatively, the formation of this type of heterodimers can be biased by genetically engineering and expressing chimeric molecules that contain isoforms, such as a RAGE isoform and another isoform, followed by the Fc-domain of hIgG, followed by either c-jun or the c-fos leucine zippers. Since these leucine zippers form predominantly heterodimers, they can be used to drive the formation of the heterodimers when desired. Chimeric proteins containing Fc regions can be engineered to include a tag with metal chelates or other epitope. The tagged domain can be used for rapid purification by metal-chelate chromatography, and/or by antibodies, to allow for detection of western blots, immunoprecipitation, or activity depletion/blocking in bioassays.

In additional examples, heterodimers can be prepared using immunoglobulin derived domains that drive the formation of dimers. Such domains include, for example, the heavy chains of IgG (Cγ1 and Cγ4), as well as the constant regions of kappa (κ) and lambda (λ) light chains of immunoglobulins. The heterodimerization of Cγ with the light chain occurs between the CH1 domain of Cγ and the constant region of the light chain (C_{L}), and is stabilized by covalent linking of the two domains via a single disulfide bridge. Alternatively, the immunoglobulin domains can include domains that are derived from T cell receptor components which drive dimerization.

Preparation of chimeric fusion proteins containing heterologous polypeptides fused to various portions of antibody-derived polypeptides (including the Fc domain) has been described (see e.g., Ashkenazi et al., (1991) PNAS 88:10535); Byrn et al., (1990) Nature 344:677). An Fc polypeptide can be a native or mutein form, as well as a truncated Fc polypeptides containing the hinge region that promotes dimerization. An exemplary Fc portion is derived from hIgG1. In some examples, the linker length of the hinge region can vary. Examples of amino acid sequences of an Fc include, but are not limited to, those set forth in SEQ ID NO: 361 and 362. An exemplary mutein Fc is set forth in SEQ ID NO:363. Such a mutein Fc is identical to the amino acid sequence set forth in SEQ ID NO:362, except amino acid 32 has been changed from Leu to Ala, amino acid 33 has been changed from Leu to Glu, and amino acid 35 has been changed from Gly to Ala. Such a mutein Fc exhibits reduced affinity for immunoglobulin receptors.

Heteromultimeric chimeric isoform fusions also can be generated utilizing protein-protein interactions between the regulatory (R) subunit of cAMP-dependent protein kinase (PKA) and the anchoring domains (AD) of A kinase anchor proteins (AKAPs, see e.g., Rossi et al., (2006) PNAS 103:6841-6846). Two types of R subunits (RI and RII) are found in PKA, each with an α and β isoform. The R subunits exist as dimers, and for RII, the dimerization domain resides in the 44 amino-terminal residues (see e.g., SEQ ID NO: 357). AKAPs, via the interaction of their AD domain, interact with the R subunit of PKA to regulate its activity. AKAPs bind only to dimeric R subunits. For example, for human RIIα, the AD binds to a hydrophobic surface formed from the 23 amino-terminal residues. An exemplary sequence of AD is AD1 set forth in SEQ ID NO:358, which is a 17 amino acid residue sequence derived from AKAP-IS, a synthetic peptide optimized for RII-selective binding. Thus, a heteromultimeric isoform polypeptide can be generated by linking (directly or indirectly) a nucleic acid encoding a polypeptide isoform, such as a RAGE isoform, with a nucleic acid encoding an R subunit sequence (i.e. SEQ ID NO:357). This results in a homodimeric molecule, due to the spontaneous formation of a dimer effected by the R subunit. In tandem, another chimeric polypeptide isoform can be generated by linking a nucleic acid encoding another polypeptide isoform to a nucleic acid sequence encoding an AD sequence. Upon co-expression of the two components, such as following co-transfection of the chimeric isoform fusion components in host cells, the dimeric R subunit provides a docking site for binding to the AD sequence, resulting in a heteromultimeric molecule. This binding event can be further stabilized by covalent linkages, such as for example, disulfide bonds. In some examples, a flexible linker residue can be fused between the nucleic acid encoding the polypeptide isoform and the multimerization domain. In another example, fusion of a nucleic acid encoding a polypeptide isoform can be to a nucleic acid encoding an R subunit containing a cysteine residue incorporated adjacent to the amino-terminal end of the R subunit to facilitate covalent linkage (see e.g., SEQ ID NO:359). Similarly, fusion of a nucleic acid encoding a partner polypeptide isoform can be to a nucleic acid encoding an AD subunit also containing incorporation of cysteine residues to both the amino- and carboxyl-terminal ends of AD (see e.g., SEQ ID NO:360).

Other multimerization domains are known to those of skill in the art and are any that facilitate the protein-protein interaction of two or more polypeptides that are separately generated and expressed as chimeric fusions. Examples of other multimerization domains that can be used to provide protein-protein interactions between two chimeric polypeptides include, but are not limited to, the barnase-barstar module (see e.g., Deyev et al., (2003) Nat. Biotechnol. 21:1486-1492); selection of particular protein domains (see e.g., Terskikh et al., (1997) PNAS 94: 1663-1668 and Muller et al., (1998) FEBS Lett. 422:259-264); selection of particular peptide motifs (see e.g., de Kruif et al., (1996) J. Biol. Chem. 271:7630-7634 and Muller et al., (1998) FEBS Lett. 432: 45-49); and the use of disulfide bridges for enhanced stability (de Kruif et al., (1996) J. Biol. Chem. 271:7630-7634 and Schmiedl et al., (2000) Protein Eng. 13:725-734).

Chimeric fusion polypeptides can be generated by fusion of nucleic acid encoding the polypeptide isoform to a multimerization domain either directly or indirectly. For example, fusion of a chimeric fusion polypeptide to a multimerization domain can be through direct linkage. Such sequences can be constructed using recombinant DNA techniques. Alternatively, fusion of a chimeric isoform polypeptide to a multimerization domain can be through indirect linkage, such as by covalent linkage using, for example, heterobifunctional crosslinking agents such as is described above.

When preparing chimeric isoform polypeptides, nucleic acids encoding an isoform, or portion thereof, of a cognate ligand or receptor is fused C-terminally to nucleic acid encoding the N-terminus of a multimerization domain, such as for example, an immunogloculin constant domain sequence, however, N-terminal fusions are also possible. Typically, where fusion is to an immunoglobulin constant domain sequence (i.e. Fc), the encoded chimeric polypeptide retains at least a functionally active hinge, CH2 and CH3 domains of the constant region of an immunoglobulin heavy chain. Fusions also can be made to the C-terminus of the Fc portion of a constant domain, or immediately N-terminal to the CH1 of the heavy chain or the corresponding region of the light chain. The resultant DNA fusion construct is expressed in appropriate host cells.

Expression of RAGE isoform heterodimers can be facilitated by co-transfection of host cells with the appropriate isoform components (i.e. nucleic acids encoding a RAGE isoform chimeric polypeptide containing a multimerization domain and/or another isoform polypeptide containing a multimerization domain). Expression thereof of each of the respective chimeric fusion polypeptides, including a RAGE chimeric fusion polypeptide, results in interaction of the multimerization domains to form stable protein-protein interaction between a first polypeptide chimeric fusion and a second polypeptide chimeric fusion. RAGE isoform heterodimers can be purified from cell lines cotransfected with the appropriate isoform components If necessary, heterodimers can be separated from homodimers using methods available to those of skill in the art. For example, limited quantities of RAGE isoform heterodimers can be recovered by passive elution from preparative, nondenaturing polyacrylamide gels. Alternatively, heterodimers can be purified using high pressure cation exchange chromatography, for example, using a Mono S cation exchange column.

Additionally, a chimeric isoform polypeptide can contain a fusion of a nucleic acid encoding a monomer of the chimeric heterodimer with a nucleic acid encoding a tag polypeptide, which provides an epitope to which an anti-tag antibody can selectively bind. Such epitope tagged forms of the chimeric heterodimer facilitate the detection of the heterodimer using a labeled antibody against the tag polypeptide. Also, the presence of the epitope tag enables the chimeric heterodimer to be readily purified by affinity purification using an anti-tag antibody. Examples of tags, include but are not limited to, the flu HA tag polypeptide and its antibody 12CA5, the c-myc tag and the 8F9, 3C7, 6E10, G4, B7, and 9E10 antibodies thereto, and the Herpes Simplex virus glycoprotein D (gD) and its antibody.

Another type of covalent modification of a chimeric heteromultimer includes linking an isoform monomer polypeptide of the heteromultimer to one of a variety of nonproteinaceous polymers, e.g., polyethylene glycol, polypropylene glycol, polyoxyalkylenes, or copolymers of polyethylene glycol and polypropylene glycol. A chimeric heteromultimer also can be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsion, nano-particles and nanocapsules), or in macroemulsions.

c. Methods of Generating and Cloning RAGE Fusions

The methods by which DNA sequences may be obtained and linked to provide the DNA sequence encoding the fusion protein are well known in the field of recombinant DNA technology. DNA for a sequence to be fused to a RAGE isoform, including, but not limited to a sequence of a RAGE isoform, a precursor signal sequence, a fusion tag, another isoform or intron-encoded portion thereof, or any other desired sequence can be generated by various methods including: synthesis using an oligonucleotide synthesizer; isolation from a target DNA such as from an organism, cell, or vector containing the sequence, by appropriate restriction enzyme digestion; or can be obtained from a target source by PCR of genomic DNA with the appropriate primers. In a PCR method, primers directed against a target sequence, such as a RAGE isoform sequence, can be engineered that contain sequences for small epitope tags, such as a myc tag, His tag, or other small epitope tag, and/or any other additional DNA sequence such as a restriction enzyme linker sequence or a protease cleavage site sequence such that the entire PCR sequence is incorporated into a target nucleic acid sequence upon PCR amplification. In an exemplary embodiment, the primer can introduce restriction enzyme sites into a RAGE isoform sequence, or other target sequence, to facilitate the cloning of the sequence into a vector.

In one example, RAGE isoform fusion sequences can be generated by successive rounds of ligating DNA target sequences, amplified by PCR, into a vector at engineered recombination site. For example, a nucleic acid sequence for a RAGE isoform, fusion tag, homologous or heterologous precursor sequence, or other desired nucleic acid sequence can be PCR amplified using primers that hybridize to opposite strands and flank the region of interest in a target DNA. Cells or tissues or other sources known to express a target DNA molecule, or a vector containing a sequence for a target DNA molecule, can be used as a starting product for PCR amplification events. The PCR amplified product can be subcloned into a vector for further recombinant manipulation of a sequence, such as to create a fusion with another nucleic acid sequence already contained within a vector, or for the expression of a target molecule.

PCR primers used in the PCR amplification also can be engineered to facilitate the operative linkage of nucleic acid sequences. For example, non-template complementary 5′ extension can be added to primers to allow for a variety of post-amplification manipulations of the PCR product without significant effect on the amplification itself. For example, these 5′ extensions can include restriction sites, promoter sequences, sequences for epitope tags, etc. In one example, for the purpose of creating a fusion sequence, sequences that can be incorporated into a primer include, for example, a sequence encoding a myc epitope tag or other small epitope tag, such that the amplified PCR product effectively contains a fusion of a nucleic acid sequence of interest with an epitope tag.

In another example, incorporation of restriction enzyme sites into a primer can facilitate subcloning of the amplification product into a vector that contains a compatible restriction site, such as by providing sticky ends for ligation of a nucleic acid sequence. Subcloning of multiple PCR amplified products into a single vector can be used as a strategy to operatively link or fuse different nucleic acid sequences. Examples of restriction enzyme sites that can be incorporated into a primer sequence can include, but are not limited to, an Xho I restriction site, an NheI restriction site, a Not I restriction site, an EcoRI restriction site, or an Xba I restriction site. Other methods for subcloning of PCR products into vectors include blunt end cloning, TA cloning, ligation independent cloning, and in vivo cloning.

The creation of an effective restriction enzyme site into a primer requires the digestion of the PCR fragment with a compatible restriction enzyme to expose sticky ends, or for some restriction enzyme sites, blunt ends, for subsequent subcloning. There are several factors to consider in engineering a restriction enzyme site into a primer so that it retains its compatibility for a restriction enzyme. First, the addition of 2-6 extra bases upstream of an engineered restriction site in a PCR primer can greatly increase the efficiency of digestion of the amplification product. Other methods that can be used to improve digestion of a restriction enzyme site by a restriction enzyme include proteinase K treatment to remove any thermostable polymerase that can block the DNA, end-polishing with Klenow or T4 DNA polymerase, and/or the addition of spermidine. An alternative method for improving digestion efficiency of PCR products also can include concatamerization of the fragments after amplification. This is achieved by first treating the cleaned up PCR product with T4 polynucleotide kinase (if the primers have not already been phosphorylated). The ends may already be blunt if a proofreading thermostable polymerase such as Pfu was used or the amplified PCR product can be treated with T4 DNA polymerase to polish the ends if a non-proofreading enzyme such as Taq is used. The PCR products can be ligated with T4 DNA ligase. This effectively moves the restriction enzyme site away from the end of the fragments and allows for efficient digestion.

Prior to subcloning of a PCR product containing exposed restriction enzyme sites into a vector, such as for creating a fusion with a sequence of interest, it is sometimes necessary to resolve a digested PCR product from those that remain uncut. In such examples, the addition of fluorescent tags at the 5′ end of a primer can be added prior to PCR. This allows for identification of digested products since those that have been digested successfully will have lost the fluorescent label upon digestion.

In some instances, the use of amplified PCR products containing restriction sites for subsequent subcloning into a vector for the generation of a fusion sequence can result in the incorporation of restriction enzyme linker sequences in the fusion protein product. Generally such linker sequences are short and do not impair the function of a polypeptide so long as the sequences are operatively linked.

The nucleic acid molecule encoding an isoform fusion protein can be provided in the form of a vector which comprises the nucleic acid molecule. One example of such a vector is a plasmid. Many expression vectors are available and known to those of skill in the art and can be used for expression of aCSR isoform, including isoform fusions. The choice of expression vector can be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

2. Targeting Agent/Targeting Agent Conjugates

RAGE isoforms also can be provided as conjugates between the isoform and another agent. The conjugate can be used to target to a receptor with which the isoform interacts and/or to another targeted receptor for delivery of isoform. Such conjugates include linkage of a RAGE isoform to a targeted agent and/or targeting agent. Conjugates can be produced by any suitable method including chemical conjugation or by expression of fusion proteins in which, for example, DNA encoding a targeted agent or targeting agent, with or without a linker region, is operatively linked to DNA encoding a RAGE isoform. Protein conjugates also can be produced by chemical coupling, typically through disulfide bonds between cysteine residues present in or added to the components, or through amide bonds or other suitable bonds. Ionic or other linkages also are contemplated.

Conjugates can contain one or more RAGE isoforms linked, either directly or via a linker, to one or more targeted agents: (RAGE isoform)n, (L)q, and (targeted agent)m in which at least one RAGE isoform is linked directly or via one or more linkers (L) to at least one targeted agent. Such conjugates also can be produced with any portion of a RAGE isoform sufficient to bind a target, such as a target cell type for treatment. Any suitable association among the elements of the conjugate and any number of elements where n, and m are integer greater than 1 and q is zero or any integer greater then 1, is contemplated as long as the resulting conjugates interacts with a targeted RAGE or targeted cell type.

Examples of a targeted agent include drugs and other cytotoxic molecules such as toxins that act at or via the cell surface and those that act intracellularly. Examples of such moieties, include radionuclides, radioactive atoms that decay to deliver, e.g., ionizing alpha particles or beta particles, or X-rays or gamma rays, that can be targeted when coupled to a RAGE isoform. Other examples include chemotherapeutics that can be targeted by coupling with an isoform. For example, geldanamycin targets proteosomes. An isoform-geldanamycin molecule can be directed to intracellular proteosomes, degrading the targeted isoform and liberating geldanamycin at the proteosome. Other toxic molecules include toxins, such as ricin, saporin and natural products from conches or other members of phylum mollusca. Another example of a conjugate with a targeted agent is a RAGE isoform coupled, for example as a protein fusion, with an antibody or antibody fragment. For example, an isoform can be coupled to an Fc fragment of an antibody that binds to a specific cell surface marker to induce killer T cell activity in neutrophils, natural killer cells, and macrophages. A variety of toxins are well known to those of skill in the art.

Conjugates can contain one or more RAGE isoforms linked, either directly or via a linker, to one or more targeting agents: (RAGE isoform)n, (L)q, and (targeting agent)m in which at least one RAGE isoform is linked directly or via one or more linkers (L) to at least one targeting agent. Any suitable association among the elements of the conjugate and any number of elements where n, and m are integer greater than 1 and q is zero or any integer greater then 1, is contemplated as long as the resulting conjugates interacts with a target, such as a targeted cell type.

Targeting agents include any molecule that targets a RAGE isoform to a target such as a particular tissue or cell type or organ. Examples of targeting agents include cell surface antigens, cell surface receptors, proteins, lipids and carbohydrate moieties on the cell surface or within the cell membrane, molecules processed on the cell surface, secreted and other extracellular molecules. Molecules useful as targeting agents include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting agents include ligands for receptors, such as proteinaceous and small molecule ligands, and antibodies and binding proteins, such as antigen-binding proteins.

Alternatively, the RAGE isoform, which specifically interacts with a particular receptor (or receptors) is the targeting agent and it is linked to targeted agent, such as a toxin, drug or nucleic acid molecule. The nucleic acid molecule can be transcribed and/or translated in the targeted cell or it can be regulatory nucleic acid molecule.

The RAGE can be linked directly to the targeted (or targeting agent) or via a linker. Linkers include peptide and non-peptide linkers and can be selected for functionality, such as to relieve or decrease steric hindrance caused by proximity of a targeted agent or targeting agent to a RAGE isoform and/or increase or alter other properties of the conjugate, such as the specificity, toxicity, solubility, serum stability and/or intracellular availability and/or to increase the flexibility of the linkage between a RAGE isoform and a targeted agent or targeting agent. Examples of linkers and conjugation methods are known in the art (see, for example, WO 00/04926). RAGE isoforms also can be targeted using liposomes and other such moieties that direct delivery of encapsulated or entrapped molecules.

3. Peptidomimetic Isoforms

Also provided are “peptidomimetic” isoforms in which one or more bonds in the peptide backbone (or other bond(s)) is (are) replaced by a bioisotere or other bond such that the resulting polypeptide peptidomimetic has improved properties, such as resistance to proteases, compared to the unmodified form.

H. Assays to Assess or Monitor RAGE Isoform Activities

Generally, the RAGE isoforms provided herein exhibit an alteration in structure and also one or more activities compared to a wildtype or predominant form of a receptor. All such isoforms are candidate therapeutics. If needed, identified isoforms can be screened using in vitro and in vivo assays to monitor or identify an activity of a RAGE isoforms and to select RAGE isoforms that exhibit such an activity or alteration in activity and/or that exhibit ligand binding or that modulate RAGE activity.

Any suitable assay can be employed, including assays exemplified herein. Numerous assays for biological activities of RAGE are known to one of skill in the art. The assays permit comparison of an activity of a RAGE isoform to an activity of a wildtype or predominant form of a RAGE receptor to identify isoforms that lack an activity. In addition, assays permit identification of isoforms that modulate the activity of a RAGE receptor. Assays for RAGE and RAGE isoforms include, but are not limited to, immunostaining and localization, ligand binding and competition assays, heparin binding, gene expression assays, ERK phosphorylation, cell proliferation assays, cord-like formation assays, cell migration assays, and neurite outgrowth assays.

Alternatively or in addition, RAGE isoforms modulate the activity of a RAGE and/or bind to or interact with RAGE ligands. Identified isoforms can be screened for such activities. Assays to screen isoforms to identify activities and functional interactions with RAGE and/or RAGE ligands are known to those of skill in the art. One of skill in the art can test a particular isoform for interaction with RAGE or a RAGE ligand and/or test to assess any change in activity compared to a RAGE. Some are exemplified herein.

1. Ligand Binding Assays and RAGE Binding Assays

RAGE binding can be assessed directly by assessing binding a RAGE or by competitive assays with an AGE or other known ligand for binding to cells known to express a RAGE.

Ligand binding can be measured directly or indirectly for one or more than one ligand. For example, the ability of a RAGE isoform to bind to AGEs can be measured using affinity column chromatography. RAGE isoforms are expressed in cells and then cell extract, semi-purified or substantially purified RAGE isoform generated from such cells is applied to an AGE column. RAGE isoforms bound to AGE can then be eluted and quantitated by immunoblotting using anti-RAGE antibodies. In another assay, immunoprecipitation is used to assess ligand binding. Cell lysates expressing a RAGE isoform are incubated with a ligand, for example, S100P. Antibodies against the ligand S100P are used to immunoprecipitate the complex. The amount of RAGE isoform in the complex is quantified and/or detected using western blotting of the immunoprecipitates with anti-RAGE antibodies. Ligand binding assays also can include binding to ligands in the presence of other molecules. For example, ligand binding can be assessed in the presence of LF-L.

2. Complexation

RAGE isoforms can be assayed for their ability to complex with other proteins. In one example, a RAGE isoform can be assessed for complexation with LF-L (lactoferrin-like AGE binding protein) using a ligand blotting assay (see e.g., Schmidt et al. 1994 J. Biol. Chem. 269: 9882-88). LF-L radiolabeled with ¹²⁵I (¹²⁵I-LF-L) is incubated with RAGE protein (isoform and/or wildtype form) immobilized on a solid support After washing, the amount of ¹²⁵I-LF-L associated with a RAGE isoform can be quantified.

Complexation also can be assessed in a competitive assay. RAGE is adsorbed onto polypropylene tubes such that it remains tightly bound to the tubes (see Schmidt et al. 1994 J. Biol. Chem. 269: 9882-88). ¹²⁵II-LF-L is added to the tubes alone or after preincubation with a RAGE isoform. After an incubation period, the tubes are washed and the amount of ¹²⁵I-LF-L binding is assessed by measuring the radioactivity associated with each tube. A comparison of the samples that were preincubated with a RAGE isoform versus no preincubation indicates whether the RAGE isoform competes effectively for binding to LF-L.

3. Gene Expression Assays

RAGE isoform modulation of gene expression can be assessed for example in cell-based assays. Cells are transformed with a RAGE cDNA or control (such as a wildtype/predominant form of RAGE and/or vector alone). After an incubation period, cells are incubated with a RAGE ligand (e.g. AGEs, S100/calgranulin) and then washed. RNA is isolated from the cells and subjected to RT-PCR assays. Using RT-PCR, and primers for genes of interest, gene expression can be compared between cells containing different RAGE isoforms, with cells expressing a wildtype/predominant form of RAGE, with and without ligand and in comparison to vector alone controls. Examples of genes whose expression can be assessed includes, but is not limited to, VEGF-A, COX-2, IL-1β, COX-1, IL-6, and NF-κB. In addition, gene expression assays can be performed in a variety of cell types to assess cell-type specific affects on signal transduction, including gene expression.

Effects on gene expression also can be monitored by measuring protein expression from such genes, such as by immunoblotting with appropriate antibodies and/or by measuring enzyme activity of expression proteins, where appropriate. For example, RAGE isoform modulation of NF-κB can be assessed using a gel-shift assay. Cells transformed with a RAGE isoform or control are incubated in the presence or absence of ligand. Cell nuclear extracts are then incubated with a radiolabeled oligonucleotide that contains one or more binding sites for NF-κB. After incubation, the extracts are subjected to non-denaturing gel electrophoresis. Visualization of the migration of the radiolabel in each of the samples is assessed and compared as a measurement of NF-κB DNA binding in the samples (see for example, Bierhaus et al. 2001 Diabetes 50:2792-2808).

Reporter gene assays also can be used to measure RAGE isoform modulation of gene expression. Cells are transformed with a promoter of interest operably linked to a reporter gene, for example an NF-κB-responsive promoter operable linked to a luciferase gene (see for example, Huttunen et al. 1999 J. Biol. Chem. 274:19919-24). The cells also are transfected with a RAGE isoform or control. The transformed cells are incubated in the presence and absence of ligand. Luciferase activity is then measured in extracts from each of the cell samples and compared. Similar assays can be performed to assess modulatory affects on any gene of interest including assessing effects on endogenous RAGE expression using a RAGE promoter.

4. Cell Proliferation Assays

Modulation of cell proliferation by RAGE isoforms can be assessed in cells transformed with a RAGE isoform. Cells, seeded at a predetermined density, such as ECV304 cells, are transformed with a RAGE cDNA or control (such as a wildtype/predominant form of RAGE and/or vector alone). After an incubation and attachment period, ligand is added and the cells are incubated again. Cell proliferation can then be assessed, for example using a 3-(4,5-dimethylthiazol-2-yl)-2,5 diphenyl-2H-tetrazolium bromide (MTT) method. (see Yonekura et al. 2003 Biochem J. 370:1097-1109).

5. ERK Phosphorylation Assays

RAGE isoforms can be assessed for their ability to stimulate ERK phosphorylation. Endothelial cells (human microvascular EC cells) expressing RAGE or a RAGE isoform are incubated in serum-free media and then AGEs are added for an incubation period. After washing, cells are solubilized and extracts subject to SDS-PAGE. Proteins are transferred to a membrane and the amount of phosphorylated ERK is assessed by immunoreactivity with an ant-phosphoERK antibody.

6. Cell Migration Assay

RAGE isoform effects on cell migration can be assessed. Cells, such as ECV304 cells, are stably transformed with a RAGE isoform cDNA or control (e.g. a wildtype/predominant form of RAGE and/or vector alone). The stably transformed cells are seeded onto plates and grown to confluence. Cells are wounded by denuding a strip of the monolayer of cells. After washing in serum free media, the cells are incubated with media containing serum and type I collagen. Cell cultures are photographed over time to monitor the rate of wound closure (i.e. cell migration into the wounded strip area).

7. Neurite Outgrowth

RAGE-mediated affects on neurite outgrowth can be assessed for a RAGE isoform by stably transforming a neuroblastoma cell line with a RAGE cDNA or control. The cells are serum starved and grown overnight on amphoterin coated glass slides. Filamentous actin is stained, for example using TRITC-phalloidin and the percentage of cells bearing neuritis is assessed and compared between samples. Cells also can be stained with an antibody against RAGE (or against a tag if tagged-RAGE is expressed, e.g. a myc tag) to assess the proportion of cells expressing a RAGE isoform that formed neurite outgrowths (see for example, Huttunen et al. 1999 J. Biol. Chem. 274:19919-24).

8. Animal Models

Assessment of a RAGE isoform on a disease or condition can be assessed in an animal model. A variety of animal models are available to diseases and conditions in which RAGE plays a role.

(a) Diabetic Vasculopathy

Diabetic vasculopathy can be assessed in a rat model. Rats are rendered diabetic by dosing with streptozocin. After 9-11 weeks of the induced diabetic condition, a RAGE isoform or a control is administered. After dosing with the RAGE isoform, tissue-blood isotope ration (TBIR) is assessed. Diabetic rats display increased vascular permeability, especially in intestine, skin and kidney compared with non-diabetic controls. The rats dosed with the RAGE isoform are compared with controls to assess the ability of the RAGE isoform. Dosage dependent effects can be assessed as well as comparisons made between isoforms and with controls including a wildtype/predominant form of RAGE and a empty vector control.

(b) Diabetic Atherosclerosis

Mice such as C57BL/6 and Balb/c strains, treated with streptozocin develop symptoms of early and non-complex atherosclerosis characteristic of human diabetes. ApoE null mice also can be tested; these mice develop spontaneous atherosclerosis symptoms on normal low-fat rodent chow. Induction of ApoE null mice with streptozocin increases the severity of the symptoms of atherosclerosis compared to untreated ApoE null mice. A RAGE isoform is administered to the mice and after a period of dosing phenotypes are assessed. Morphometric analysis can be performed on serial sections of the aortic sinus and severity and numbers of lesions (including fibrous caps and extensive monocyte and smooth muscle infiltration) is assessed. Comparisons of mice administered a RAGE isoform or control are compared to assess the ability of the RAGE isoform to arrest or reduce lesion accumulation and suppress diabetic atherosclerosis.

(c) Diabetic Inflammatory Bone Loss

Diabetic mice can be induced to display increased bone lass in gingival tissues, similar to gingivitis-periodontis seen in human diabetic subjects. C57BL/6J mice are rendered diabetic by administration of streptozotocin. One month after treatment, the mice are inoculated with the human periodontal pathogen Porphyromonas gingivalis by local oral-anal gavage and swabbing. The extent of bone loss is assessed by comparing serial sections of mandibular alveolar bone. Induction of inflammatory response is monitored through assessment of tumor necrosis factor (TNF), interleukin-6 (IL-6), matrix metalloproteinase 3 and 9 antigens (MMP3 and MMP9) using western blotting and/or enzyme assays. Diabetic mice have increased inflammatory responses and bone loss compared to non-diabetic mice. The affect of a RAGE isoform on the suppression of bone loss and/or inflammatory response is assessed by administration of a RAGE isoform directly after pathogen infection. Comparison of mice treated with a RAGE isoform to controls (vehicle alone and or mice treated with a wildtype/predominant form of RAGE) indicates the affect of the RAGE isoform on suppression of bone loss and/or proinflammatory responses.

(d) Autoimmune Diabetes

Mouse models of autoimmune diabetes can be constructed (see for example, Chen et al. 2004 J. Immunology 173 1399-1405). Splenocytes from diabetic mice are transferred into NOD/scid mice. After about 1-½ month, most of the mice receiving the transfer are diabetic. To assess the affect on a RAGE isoform for its ability to suppress diabetes, mice receiving the splenocytes also are treated with a RAGE isoform (e.g. 50 μg/day) or a control (e.g. mouse albumin). Onset of diabetes is compared between the RAGE isoform-treated and control mice.

Suppression of recurrent autoimmune diabetes also can be assessed by constructing a mouse diabetic model using islet grafts. Diabetic immune-competent NOD mice are grafted with islets (e.g. 500 islets) underneath the kidney capsule. Animals with the graft usually have disease recurrence within 30 days. To assess the ability of a RAGE isoform to suppress disease recurrence, a RAGE isoform is administered following islet transplant and disease symptoms, including blood glucose level are compared with controls.

I. Preparation, Formulation and Administration of RAGE Isoforms and RAGE Isoform Compositions

RAGE isoforms and RAGE isoform compositions can be formulated for administration by any route known to those of skill in the art including intramuscular, intravenous, intradermal, intraperitoneal injection, subcutaneous, epidural, nasal oral, rectal, topical, inhalational, buccal (e.g., sublingual), and transdermal administration or any route. RAGE isoforms can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case depends on a variety of factors, such as the nature of the disease, the progress of the disease, the severity of the disease the particular composition which is used.

Various delivery systems are known and can be used to administer RAGE isoforms, such as but not limited to, encapsulation in liposomes, microparticles, microcapsules, recombinant cells capable of expressing the compound, receptor mediated endocytosis, and delivery of nucleic acid molecules encoding RAGE isoforms such as retrovirus delivery systems.

Pharmaceutical compositions containing RAGE isoforms can be prepared. Generally, pharmaceutically acceptable compositions are prepared in view of approvals for a regulatory agency or other prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to a subject or patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non-toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly (alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of RAGE isoforms can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release formulations and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles also can be employed with RAGE isoform administration. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 .ANG., containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

Administration methods can be employed to decrease the exposure of RAGE isoforms to degradative processes, such as proteolytic degradation and immunological intervention via antigenic and immunogenic responses. Examples of such methods include local administration at the site of treatment. Pegylation of therapeutics has been reported to increase resistance to proteolysis; increase plasma half-life, and decrease antigenicity and immunogenicity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003)Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects). administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

A RAGE isoform is included in the pharmaceutically acceptable carrier in an amount sufficient to exert a therapeutically useful effect in the absence of undesirable side effects on the subject or patient treated. Therapeutically effective concentration can be determined empirically by testing the compounds in known in vitro and in vivo systems, such as the assays provided herein.

The concentration a RAGE isoform in the composition depends on absorption, inactivation and excretion rates of the complex, the physicochemical characteristics of the complex, the dosage schedule, and amount administered as well as other factors known to those of skill in the art. The amount of a RAGE isoform to be administered for the treatment of a disease or condition, for example cancer, autoimmune disease and infection can be determined by standard clinical techniques. In addition, in vitro assays and animal models can be employed to help identify optimal dosage ranges. The precise dosage, which can be determined empirically, can depend on the route of administration and the seriousness of the disease. Suitable dosage ranges for administration can range from about 0.01 pg/kg body weight to 1 mg/kg body weight and more typically 0.05 mg/kg to 200 mg/kg RAGE isoform: patient (subject) weight.

A RAGE isoform can be administered at once, or can be divided into a number of smaller doses to be administered at intervals of time. RAGE isoforms can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. The mode of administration of the composition containing the polypeptides as well as compositions containing nucleic acids for gene therapy, includes, but is not limited to intralesional, intraperitoneal, intramuscular and intravenous administration. Also included are infusion, intrathecal, subcutaneous, liposome-mediated, depot-mediated administration. Also included, are nasal, ocular, oral, topical, local and otic delivery. Dosages can be empirically determined and depend upon the indication, mode of administration and the subject. Exemplary dosages include from 0.1, 1, 10, 100, 200 and more mg/day/kg weight of the subject.

J. In Vivo Expression of RAGE Isoforms and Gene Therapy

Rage isoforms can be delivered to cells and tissues by expression of nucleic acid molecules. RAGE isoforms can be administered as nucleic acid molecules encoding a RAGE isoform, including ex vivo techniques and direct in vivo expression.

1. Delivery of Nucleic Acids

Nucleic acids can be delivered to cells and tissues by any method known to those of skill in the art.

a. Vectors—Episomal and Integrating

Methods for administering RAGE isoforms by expression of encoding nucleic acid molecules include administration of recombinant vectors. The vector can be designed to remain episomal, such as by inclusion of an origin of replication or can be designed to integrate into a chromosome in the cell. RAGE isoforms also can be used in ex vivo gene expression therapy using non-viral vectors. For example, cells can be engineered to express a RAGE isoform, such as by integrating a RAGE isoform encoding-nucleic acid into a genomic location, either operatively linked to regulatory sequences or such that it is placed operatively linked to regulatory sequences in a genomic location. Such cells then can be administered locally or systemically to a subject, such as a patient or subject in need of treatment.

Viral vectors, include, for example adenoviruses, herpes viruses, retroviruses and others designed for gene therapy can be employed, The vectors can remain episomal or can integrate into chromosomes of the treated subject. A RAGE isoform can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient or subject in need of treatment, and transduced with a RAGE isoform-expressing adenovirus vector. After a suitable culturing period, the transduced cells are administered to a subject, locally and/or systemically. Alternatively, RAGE isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell.

b. Artificial Chromosomes and Other Non-Viral Vector Delivery Methods

The nucleic acid molecules can be introduced into artificial chromosomes and other non-viral vectors. Artificial chromosomes (see, e.g., U.S. Pat. No. 6,077,697 and PCT International PCT application No. WO 02/097059) can be engineered to encode and express the isoform.

c. Liposomes and Other Encapsulated Forms and Administration of Cells Containing the Nucleic Acids

The nucleic acids can be encapsulated in a vehicle, such as a liposome, or introduced into a cells, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

2. In Vitro and Ex Vivo Delivery

For ex vivo and in vivo methods, nucleic acid molecules encoding the RAGE isoform is introduced into cells that are from a suitable donor or the subject to the treated. Cells into which a nucleic acid can be introduced for purposes of therapy include, for example, any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For ex vivo treatment, cells from a donor compatible with the subject to be treated or the subject to be treated cells are removed, the nucleic acid is introduced into these isolated cells and the modified cells are administered to the subject. Treatment includes direct administration, such as or, for example, encapsulated within porous membranes, which are implanted into the patient or subject (see, e.g. U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol) electroporation, microinjection, cell fusion, DEAE-dextran, and calcium phosphate precipitation methods. Methods of DNA delivery can be used to express RAGE isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of a RAGE isoform can be linked to expression of additional molecules. For example, expression of a RAGE isoform can be linked with expression of a cytotoxic product such as in an engineered virus or expressed in a cytotoxic virus. Such viruses can be targeted to a particular cell type that is a target for a therapeutic effect. The expressed a RAGE isoform can be used to enhance the cytotoxicity of the virus.

In vivo expression of a RAGE isoform can include operatively linking a RAGE isoform encoding nucleic acid molecule to specific regulatory sequences such as a cell-specific or tissue-specific promoter. RAGE isoforms also can be expressed from vectors that specifically infect and/or replicate in target cell types and/or tissues. Inducible promoters can be use to selectively regulate RAGE isoform expression.

3. Systemic, Local and Topical Delivery

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating can be targeted cells for in vivo expression of RAGE isoforms. Cells used for in vivo expression of an isoform also include cells autologous to the patient or subject. Such cells can be removed from a patient or subject, nucleic acids for expression of a RAGE isoform introduced, and then administered to a patient or subject such as by injection or engraftment.

K. RAGE and Angiogenesis

RAGE is involved in angiogenesis (see FIG. 1 and definitions and discussion above). For example AGE-RAGE interaction elicits angiogenesis through transcriptional activation of the VEGF gene via NF-κB and AP-1 factor. Modulation of RAGE activation can increase or decrease or alter angiogenic processes. Angiogenesis is a process by which new blood vessels are formed. It occurs for example, in a healthy body for healing wounds and for restoring blood flow to tissues after injury or insult. In females, angiogenesis also occurs during the monthly reproductive cycle to rebuild the uterus lining, to mature the egg during ovulation and during pregnancy to build the placenta. Angiogenesis is controlled through a series of “on” and “off” switches. The main “on” switches are known as angiogenesis-stimulating growth factors. The main “off switches” are known as angiogenesis inhibitors. When angiogenic growth factors are produced in excess of angiogenesis inhibitors, the balance is tipped in favor of blood vessel growth. When inhibitors are present in excess of stimulators, angiogenesis is stopped. A healthy body maintains a balance of angiogenesis modulators. Angiogenic growth factors are known. These include, for example, angiogenin, angiopoietin-1, Del-1, fibroblast growth factors: acidic (aFGF) and basic (bFGF), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

1. Angiogenesis and Disease

Cellular receptors for angiogenic factors (positive and negative) can act as points of intervention in multiple disease processes, for example, in diseases and conditions where the balance of angiogenic growth factors has been altered and/or the amount or timing of angiogenesis is altered. For example, in some situations ‘too much’ angiogenesis can be detrimental, such as angiogenesis that supplies blood to tumor foci, in inflammatory responses and other aberrant angiogenic-related conditions. The growth of tumors, or sites of proliferation in chronic inflammation, generally requires the recruitment of neighboring blood vessels and vascular endothelial cells to support their metabolic requirements. This is because the diffusion is limited for oxygen in tissues. Exemplary conditions that require angiogenesis include, but are not limited to solid tumors and hematologic malignancies such as lymphomas, acute leukemia, and multiple myeloma, where increased numbers of blood vessels are observed in the pathologic bone marrow.

A critical element in the growth of primary tumors and formation of metastatic sites is the angiogenic switch: the ability of the tumor or inflammatory site to promote the formation of new capillaries from preexisting host vessels. The angiogenic switch, as used in this context, refers to disease-associated angiogenesis required for the progression of cancer and inflammatory diseases, such as rheumatoid arthritis. It is a switch that activates a cascade of physiological activities that finally result in the extension of new blood vessels to support the growth of diseased tissue. Stimuli for neo-angiogenesis include hypoxia, inflammation, and genetic lesions in oncogenes or tumor suppressors that alter disease cell gene expression.

Angiogenesis also play a role in inflammatory diseases. These diseases have a proliferative component, similar to a tumor focus. In rheumatoid arthritis, one component of this is characterized by aberrant proliferation of synovial fibroblasts, resulting in pannus formation. The pannus is composed of synovial fibroblasts which have some phenotypic characteristics with transformed cells. As a pannus grows within the joint it expresses many proangiogenic signals, and experiences many of the same neo-angiogenic requirements as a tumor. The need for additional blood supply, neoangiogenesis, is critical. Similarly, many chronic inflammatory conditions also have a proliferative component in which some of the cells composing it may have characteristics usually attributed to transformed cells.

Another example of a condition involving excess angiogenesis is diabetic retinopathy (Lip et al. Br J Ophthalmology 88: 1543, 2004)). Diabetic retinopathy has angiogenic, inflammatory and proliferative components; overexpression of VEGF, and angiopoietin-2 are common. This overexpression is likely required for disease-associated remodeling and branching of blood vessels, which then supports the proliferative component of the disease.

2. The Angiogenic Process

Angiogenesis includes several steps, including the recruitment of circulating endothelial cell precursors (CEPs), stimulation of new endothelial cell (EC) growth by growth factors, the degradation of the ECM by proteases, proliferation of ECs and migration into the target, which could be a tumor site or another proliferative site caused by inflammation. This results in the eventual formation of new capillary tubes. Such blood vessels are not necessarily normal in structure. They may have chaotic architecture and blood flow. Due to an imbalance of angiogenic regulators such as vascular endothelial growth factor, (VEGF) and angiopoietins, the new vessels supplying tumorous or inflammatory sites are tortuous and dilated with an uneven diameter, excessive branching, and shunting. Blood flow is variable, with areas of hypoxia and acidosis leading to the selection of variants that are resistant to hypoxia-induced apoptosis (often due to the loss of p53 expression); and enhanced production of proangiogenic signals. Disease-associated vessel walls have numerous openings, widened interendothelial junctions, and discontinuous or absent basement membrane; this contributes to the high vascular permeability of these vessels and, together with lack of functional lymphatics/drainage, causes interstitial hypertension. Disease-associated blood vessels may lack perivascular cells such as pericytes and smooth muscle cells that normally regulate vasoactive control in response to tissue metabolic needs. Unlike normal blood vessels, the vascular lining of tumor vessels is not a homogenous layer of ECs but often consists of a mosaic of ECs and tumor cells; the concept of cancer cell-derived vascular channels, which may be lined by ECM secreted by the tumor cells, is referred to as vascular mimicry.

A similar situation occurs where blood vessels rapidly invade sites of acute inflammation. The ECs of angiogenic blood vessels are unlike quiescent ECs found in adult vessels, where only 0.01% of ECs are dividing. During tumor angiogenesis, ECs are highly proliferative and express a number of plasma membrane proteins that are characteristic of activated endothelium, including growth factor receptors and adhesion molecules such as integrins. Tumors utilize a number of mechanisms to promote their vascularization, and in each case they subvert normal angiogenic processes to suit this purpose. For this reason, increased production of angiogenic factors, both proliferative with respect to endothelium; and structural (allowing for increased branching of the neovasculature) are likely to occur in disease foci, as in cancer or chronic inflammatory disease.

3. Cell Surface Receptors in Angiogenesis

Cell surface receptors including RTKs, and their ligands play a role in the regulation of angiogenesis (see for example, FIG. 1). Angiogenic endothelium expresses a number of receptors not found on resting endothelium. These include receptor tyrosine kinases (RTK) and integrins that bind to the extracellular matrix and mediate endothelial cells adhesion, migration, and invasion.

Endothelial cells (ECs) also express RTK (i.e., the FGF and PDGF receptors) that are found on many other cell types. Functions mediated by activated RTK include proliferation, migration, and enhanced survival of endothelial cells, as well as regulation of the recruitment of perivascular cells and bloodborne circulating endothelial precursors and hematopoietic stem cells to the tumor. One example of a CSR involved in angiogenesis is VEGFR. VEGFR1 receptors and VEGF-A ligand are involved in cell proliferation, migration and differentiation in angiogenesis. VEGF-A is a heparin-binding glycoprotein with at least four isoforms that regulate blood vessel formation by binding to RTKs, VEGFR1 and VEGFR2. These VEGF receptors are expressed on all ECs in addition to a subset of hematopoietic cells. VEGFR2 regulates EC proliferation, migration, and survival, while VEGFR1 may act as an antagonist of R1 in ECs but also can plays a role in angioblast differentiation during embryogenesis.

Additional signaling pathways also are involved in angiogenesis. The angiopoietin, Ang1, produced by stromal cells, binds to the EC RTK Tie-2 and promotes the interaction of ECs with the ECM and perivascular cells, such as pericytes and smooth muscle cells, to form tight, non-leaky vessels. PDGF and basic fibroblast growth factor (bFGF) help to recruit these perivascular cells. Ang1 is required for maintaining the quiescence and stability of mature blood vessels and prevents the vascular permeability normally induced by VEGF and inflammatory cytokines.

Proangiogenic cytokines, chemokines, and growth factors secreted by stromal cells or inflammatory cells make important contributions to neovascularization, including bFGF, transforming growth factor-alpha, TNF-alpha, and IL-8. In contrast to normal endothelium, angiogenic endothelium overexpresses specific members of the integrin family of ECM-binding proteins that mediate EC adhesion, migration, and survival. Integrins mediate spreading and migration of ECs and are required for angiogenesis induced by VEGF and bFGF, which in turn can upregulate EC integrin expression. EC adhesion molecules can be upregulated (i.e., by VEGF, TNF-alpha). VEGF promotes the mobilization and recruitment of circulating endothelial cell precursors (CEPs) and hematopoietic stem cells (HSCs) to tumors where they colocalize and appear to cooperate in neovessel formation. CEPs express VEGFR2, while HSCs express VEGFR1, a receptor, or VEGF and PIGF. Both CEPs and HSCs are derived from a common precursor, the hemangioblast. CEPs are thought to differentiate into ECs, whereas the role of HSC-derived cells (such as tumor-associated macrophages) may be to secrete angiogenic factors required for sprouting and stabilization of ECs (VEGF, bFGF, angiopoietins) and to activate MMPs, resulting in ECM remodeling and growth factor release. In mouse tumor models and in human cancers, increased numbers of CEPs and subsets of VEGFR1 or VEGFR-expressing HSCs can be detected in the circulation, which may correlate with increased levels of serum VEGF.

4. Cell Surface Receptors in Tumors

Tumor vessels appear to be more dependent on VEGFR signaling for growth and survival than normal ECs. Tumors secrete trophic angiogenic molecules, such as VEGF family of endothelial growth factors, that induce the proliferation and migration of host ECs into the tumor. Sprouting in normal and pathogenic angiogenesis is regulated by three families of transmembrane RTKs expressed on ECs and their ligands—VEGFs, angiopoietins, and ephrins, which are produced by tumor cells, inflammatory cells, or stromal cells in the microenvironment of the disease site. Tumor or inflammatory disease-associated angiogenesis is a complex process involving many different cell types that proliferate, migrate, invade, and differentiate in response to signals from microenvironment. Endothelial cells (ECs) sprout from host vessels in response to VEGF, bFGF, Ang2, and other proangiogenic stimuli. Sprouting is stimulated by VEGF/VEGFR2, Ang2/Tie-2, and integrin/extracellular matrix (ECM) interactions. Bone marrow-derived circulating endothelial precursors (CEPs) migrate to the tumor in response to VEGF and differentiate into ECs, while hematopoietic stem cells differentiate into leukocytes, including tumor/disease site-associated macrophages that secrete angiogenic growth factors and produce MMPs that remodel the ECM and release bound growth factors.

When tumor cells arise in or metastasize to an avascular area, they grow to a size limited by hypoxia and nutrient deprivation. This condition, also likely to occur in other localized proliferative diseases, leads to the selection of cells that produce angiogenic factors. Hypoxia, a key regulator of tumor angiogenesis, causes the transcriptional induction of the gene(s) encoding VEGF by a process that involves stabilization of the transcription factor hypoxia-inducible factor (HIF)1. Under normoxic conditions, EC HIF-1 levels are maintained at a low level by proteasome-mediated destruction regulated by a ubiquitin E3-ligase encoded by the VHL tumor-suppressor locus. However, under hypoxic conditions, the HIF-1 protein is not hydroxylated and association with VHL does not occur; therefore HIF-1 levels increase, and target genes including VEGF, nitric oxide synthetase (NOS), and Ang2 are induced. Loss of the VHL genes, as occurs in familial and sporadic renal cell carcinomas, also results in HIF-1 stabilization and induction of VEGF. Most tumors have hypoxic regions due to poor blood flow, and tumor cells in these areas stain positive for HIF-1 expression. These are conditions that lead to the de novo formation of blood vessels from differentiating endothelial cells, as occurs during embryonic development) and angiogenesis under normal (wound healing, corpus luteum formation) and pathologic processes (tumor angiogenesis, inflammatory conditions such as rheumatoid arthritis).

For diseased cell-derived VEGF, such as may be produced by a growing tumor focus or by pannus formation in rheumatoid arthritis, to initiate sprouting from host vessels, the stability conferred by the Ang1/Tie2 pathway must be perturbed; this occurs by the secretion of Ang2 by ECs that are undergoing active remodeling. Ang2 binds to Tie2 and is a competitive inhibitor of Ang1 action: under the influence of Ang2, preexisting blood vessels become more responsive to remodeling signals, with less adherence of ECs to stroma and associated perivascular cells and more responsiveness to VEGF. Therefore, Ang2 is required at early stages of neoangiogenesis for destabilizing the vasculature by making host ECs more sensitive to angiogenic signals. Since tumor ECs are blocked by Ang2, there is no stabilization by the Ang1/Tie2 interaction, and tumor blood vessels are leaky, hemorrhagic, and have poor association of ECs with underlying stroma. Sprouting tumor ECs express high levels of the transmembrane protein Ephrin-B2 and its receptor, the RTK EPH whose signaling works with the angiopoietins during vessel remodeling. During embryogenesis, EPH receptors are expressed on the endothelium of primordial venous vessels while the transmembrane ligand ephrin-B2 is expressed by cells of primordial arteries; the reciprocal expression may regulate differentiation and patterning of the vasculature.

Development of tumor lymphatics also is associated with expression of cell surface receptors, including VEGFR3 and its ligands VEGF-C and VEGF-D. The role of these vessels in tumor cell metastasis to regional lymph nodes remains to be determined, since, as discussed above, interstitial pressures within tumors are high and most lymphatic vessels may exit in a collapsed and nonfunctional state. However, VEGF-C levels in primary human tumors, including lung, prostate, and colorectal cancers, correlate significantly with metastasis to regional lymph nodes, and therefore it is possible that expression of VEGF-C,D/R3 may contribute to disease spreading by maintaining an exit for tumor cells from the primary site to lymph nodes and beyond.

5. RAGE and RAGE Ligands in Angiogenesis

Advanced glycation end products (AGEs) are the result of a nonenzymatic reaction of reducing sugars with primary amino groups of proteins (Maillard reaction). They accumulate in various tissues in the course of aging. Because AGEs induce protein cross-links and oxidative stress (radicals) within cells and tissues, they have been implicated in the development of many degenerative diseases. Binding of AGEs to their cognate receptors (RAGE) induces the release of profibrotic cytokines, such as TGF-beta or proinflammatory cytokines, such as TNF-alpha or IL-6 (Simm et al., Ann. NY Acad. Sci. 1019: 228, 2004). AGEs internalized by heart vasculature, eye vasculature and other sites to create brittle and leaky blood vessels (see FIG. 1).

One of the targets that is adversely affected by AGEs is the pericyte, which provides support for stable and developing vasculature (reviewed by Stitt, Br J. Ophthalmol. 85: 746, 2001). In microvasculature, pericytes regulate growth and also serve to protect ECs. Pericyte protective functions include preserving the prostracyclin-producing ability of ECs and protecting ECs against lipid-peroxide induced injury. Hence, pericytes play a role in the maintenance of microvasculature homeostasis. AGEs including glycer-AGE and glycol-AGEs can induce apoptotic death in pericytes (Okamoto et al. (2002) FASEB J. 16(14):1928-30). The induction of apoptosis is mediated through RAGE. The apoptosis of pericytes can relive restriction on pericyte growth, contributing to stimulation of angiogenesis. Additionally, AGE stimulates VEGF production in pericytes and ECs. The upregulation of VEGF by AGE involves transcriptional activation of NF-κB and AP-1 transcription factors, both sensitive to the redox state of the cell. Increased VEGF in vascular wall cells also participates in stimulation of angiogenesis.

6. RAGE Isoforms and Angiogenesis

Modulation of angiogenesis can be used to treat diseases and conditions in which angiogenesis plays a role. For example, angiogenesis inhibitors can function by targeting the critical molecular pathways involved in EC proliferation, migration, and/or survival, many of which are unique to the activated endothelium in tumors. Inhibition of growth factor and adhesion-dependent signaling pathways can induce EC apoptosis with concomitant inhibition of tumor growth. ECs comprising the tumor vasculature are genetically stable and do not share genetic changes with tumor cells; the EC apoptosis pathways are therefore intact. Each EC of a tumor vessel helps provide nourishment to many tumor cells, and although tumor angiogenesis can be driven by a number of exogenous proangiogenic stimuli, experimental data indicate that blockade of a single growth factor (e.g., VEGF) can inhibit tumor-induced vascular growth. Because tumor blood vessels are distinct from normal ones, they may be selectively destroyed without affecting normal vessels. Additionally, reduction of AGEs and/or reduction of the effects of AGEs can inhibit or reduce angiogenesis. Agents which reduce the circulating levels of AGE molecules subjects can have a therapeutic effect. For example, reduction of AGEs can be used to treat diabetic subjects and patients who have angiogenic and vascular conditions.

Because cell surface receptors such as RAGE are involved in the regulation of angiogenesis, they can be therapeutic targets for treatment of diseases and conditions involving angiogenesis. Provided herein are RAGE isoforms that can modulate one or more steps in the angiogenic process. Exemplary steps in the angiogenesis pathway that are targets for RAGE isoforms are shown in FIG. 1. RAGE isoforms can be administered singly, in parallel or in other combinations. These isoforms can reduce or inhibit the level of circulating AGEs and/or reduce the effects of circulating AGEs in angiogenesis. RAGE isoforms can “scavenge” the circulating AGEs, thus preventing them from stimulating RAGE. RAGE isoforms also can act as negatively acting ligand that interacts with and/or inactivates the RAGE receptor, preventing circulating AGEs from stimulating the receptor and thereby inducing angiogenesis.

L. Exemplary Treatments with RAGE Isoforms

Provided herein are methods of treatment with RAGE isoforms for diseases and conditions. RAGE isoforms can be used in the treatment of a variety of diseases and conditions, including those described herein. Treatment can be effected by administering by suitable route formulations of the polypeptides, which can be provided in compositions as polypeptides and can be linked to targeting agents, for targeted delivery or encapsulated in delivery vehicles, such as liposomes. Alternatively, nucleic acids encoding the polypeptides can be administered as naked nucleic acids or in vectors, particularly gene therapy vectors. Such gene therapy can be effected ex vivo by removing cells from a subject, introducing the vector or nucleic acid into the cells and then reintroducing the modified cells. Gene therapy also can be effect in vivo by directly administering the nucleic acid or vector.

Treatments using the RAGE isoforms provided herein, include, but are not limited to treatment of diabetes-related diseases and conditions including periodontal, autoimmune, vascular, and tubulointerstitial diseases. Treatments using the RAGE isoforms also include treatment of ocular disease including macular degeneration, cardiovascular disease, neurodegenerative disease including Alzheimer's disease, inflammatory diseases and conditions including rhematoid arthritis, and diseases and conditions associated with cell proliferation including cancers. Exemplary treatments and preclinical studies are described for treatments and therapies with RAGE isoforms. Such descriptions are meant to be exemplary only and are not limited to a particular RAGE isoform. One of skill in the art can assess based on the type of disease to be treated, the severity and course of the disease, whether the molecule is administered for preventive or therapeutic purposes, previous therapy, the patient's or subject's clinical history and response to therapy, and the discretion of the attending physician appropriate dosage of a molecule to administer.

1. Age-Related Macular Degeneration

RAGE isoforms including, but not limited to, RAGE isoforms described herein such as polypeptides that contain sequences of amino acids set forth in any of SEQ ID NOS: 10-14, can be used in treatment of ocular diseases and conditions, including age-related macular degeneration. Age-related macular degeneration is associated with vision loss resulting from accumulated macular drusen, extracellular deposits in Brusch's membrane, and retinal pigment epithelium (RPE) dysfunction due to degenerative cellular and molecular changes in RPE and photoreceptors overlying the macular drusen. The cellular and molecular changes occurring in the RPE, in part due to oxidative stress in the aging eye, include altered expression of genes for cytokines, matrix organization, cell adhesion, and apoptosis resulting in the possible induction of a focal inflammatory response at the RPE-Bruch's membrane border. For example, oxidative stress induces the accumulation of RAGE ligands in the RPE and photoreceptor layers in early age-related macular degeneration. The accumulated RAGE ligands stimulate RAGE-expressing RPE cells to induce a variety of inflammatory events including NFκB nuclear localization, apoptosis, and most importantly the upregulation of the RAGE receptor itself initiating a positive feedback loop sustained by continued ligand availability. The chronic activation induced by the ligand/RAGE-mediated signaling contributes to disease progression in age-related macular degeneration. Treatment of early stage age-related macular generation with RAGE isoforms, including one or more of the isoforms set forth as SEQ ID NOS: 10-14 can ameliorate one or more symptoms of the disease.

2. Diabetes Related Diseases

RAGE isoforms including, but not limited to RAGE isoforms described herein such as polypeptides that contain sequences of amino acids set forth in any of SEQ ID NOS: 10-14, can be used to treat diabetes-related disease conditions such as vascular disease, periodontal disease, and autoimmune disease. Diabetes can occur by two main forms: type 1 diabetes is characterized by a progressive destruction of pancreatic β-islet cells which results in insulin deficiency; type 2 diabetes is characterized by an increased resistance and/or deficient secretion of insulin leading to hyperglycemia. Complications which result from hyperglycemia, such as myocardial infarction, stroke, and amputation of digits or limbs, can result in morbidity and mortality. Hyperglycemia results in sustained accumulation of RAGE ligands and signaling of RAGE by its ligands contributes to enhanced expression of the RAGE receptor in the diabetic tissue and chronic ligand-mediated RAGE signaling.

a. Vascular Disease

RAGE isoforms can be used to treat diabetes-related vascular disease, including both macrovascular and microvascular disease. Hyperglycemia occurring in type 2 diabetes results in chronic vascular injury characterized by a variety of macrovascular perturbations including the development of atherosclerotic plaques, enhanced proliferation of vascular smooth muscle, production of extracellular matrix, and vascular inflammation. Vascular inflammation can be caused and exacerbated by engagement of RAGE by its ligands leading to chronic vascular inflammation, accelerated atherosclerosis, and exaggerated restenosis after revascularization procedures. RAGE isoforms can be employed to block the ligation of RAGE by its ligands to suppress the vascular complications of diabetes. For example, in animal models of diabetes-associated hyperpermeability, treatment of animals with soluble RAGE isoform can lead to near normalization of tissue permeability. In another example of diabetes-related vascular disease, animal models of hyperlipidemia, such as ApoE −/− mice or LDL receptor −/− mice, that have been induced to develop diabetes, display increased accumulation of RAGE ligands and enhanced expression of RAGE. Treatment of diabetic mice with a soluble RAGE isoform can diminish diabetes-related atherogenesis as evidenced by reduced atherosclerotic lesion-area size and decreased levels of tissue factor, VCAM-1, and NFκB compared with vehicle-treated mice. Treatment with RAGE isoforms to block diabetic atherosclerosis can be given any time during disease progression including after establishment of atherosclerotic plaques.

Diabetes-related vascular disease also can manifest in the microvasculature affecting the eyes, kidney, and peripheral nerves. Importantly, renal disease accounts for the largest percentage of mortality of any diabetes-specific complication. RAGE isoforms can be used to treat diabetes-related vascular disease, including kidney disease. For example, in a mouse model of diabetes, insulin-resistant db/db mouse, RAGE is upregulated in the glomerulus of the kidney particularly in the podocyte cells and likewise, RAGE-ligand expressing mononuclear phagocytes also are accumulated in the glomerulus. Treatment of db/db mice with a soluble RAGE isoform blocks VEGF expression, a factor known to mediate hyperpermeability and recruitment of mononuclear phagocytes into the glomerulus. Further treatment with RAGE isoforms also decrease glomerular and mesangial expansion and decrease the albumin excretion rate.

RAGE isoforms also can be used to treat diabetes-related vascular disease associated with wound healing. Chronic wound healing is often associated with diabetes and can lead to complications such as infection and amputation. Using the db/db mouse model of type 2 diabetes, a wound healing model can be established by performing full-thickness excisional wounds to generate chronic ulcers. In such a model, the levels of RAGE and its ligands are enhanced. Treatment of mice with a soluble RAGE isoform can increase wound closure by suppressing levels of cytokines including IL-6, TNF-α, and MMP-2,3, and 9. This reduction in cytokine levels contributes to reduced chronic inflammation and ultimately enhances the generation of a thick, well-vascularized granulation tissue and increased levels of VEGF and PDGF-B.

b. Periodontal Disease

RAGE isoforms, such as polypeptides that contain sequences of amino acids set forth in any of SEQ ID Nos. 10-14, can be used to treat diabetes-related periodontal disease. Diabetes is a risk factor for the development of periodontal disease due to multiple factors including, for example, impaired host defenses upon invasion of bacterial pathogens, and exaggerated inflammatory responses once infection is established. An inappropriate immune response can lead to alveolar bone loss characteristic of periodontal disease by multiple mechanisms including, for example, impaired recruitment and function of neutrophils after infection by pathogenic bacteria, diminished generation of collagen and exaggerated collagenolytic activity, genetic predisposition, and mechanisms that lead to an enhanced inflammatory response such as, for example, sustained signaling by RAGE. RAGE and its ligands are accumulated in multiple cell types in the diabetic gingiva in patients and subjects with gingivitis-periodontitis including the endothelium and infiltrating mononuclear phagocytes. A diabetic mouse model using streptozotocin to induce diabetes, followed by inoculation of mice with the human periodonatal pathogen Porphyromonas gingivalis, can be used as a model of periodonatal disease. Mice treated with a RAGE isoform, such as by once daily intraperitoneal injections immediately following inoculation with P. gingivalis for 2 months, can be observed for periodontal disease by assessing the degree of alveolar bone loss. Reduction of cytokines and matrix metalloproteinases, such as IL-6, TNF-α, MMP-2,3,9, which are implicated in the destruction on non-mineralized connective tissue and bone, also can be observed following treatment with a RAGE isoform compared to a vehicle control.]

c. Endometriosis

Rage isoforms provided herein can be employed for treatment of endometriosis which involves angiogenesis and neovascularization.

3. Autoimmune Disease

Type I diabetes is an autoimmune disease characterized by destruction of β-islet cells, the cells that produce insulin. Type I diabetes develops when the immune system recognizes proteins on the surface of β-cells and is characterized by an inflammatory process known as insulitis where immune cells migrate into and form clusters around the pancreatic islets. Proteins, such as for example RAGE and other inflammatory mediators, contribute to the development of autoimmunity characteristic of diabetes. Mouse models of autoimmune diabetes can be developed by transfer of T lymphocytes from diabetic mice to näive mice on a NOD/SCID genetic background (i.e. devoid of T lymphocytes) or by transfer of syngenic islet cells onto a diabetic, immune-competent NOD host. Treatment of autoimmune diabetic mice with RAGE isoforms including, but not limited RAGE isoforms described herein such as polypeptides that contain sequences of amino acids set forth in any of SEQ ID NOS: 10-14, can reduce the development of insulitis by regulating the differentiation of T cells in response to antigen stimulation. Specifically treatment with RAGE isoforms can decrease, for example, the expression of inflammatory cytokines thought to be directly involved in β-cell destruction, such as TNF-α and IL-1β, and can increase other immunoregulatory cytokines, such as IL-10 and TGF-β.

Other autoimmune diseases amenable to treatment with RAGE isoforms include multiple sclerosis. Multiple sclerosis is a neuroinflammatory disorder of the central nervous system (CNS) in which T cells that are reactive with major components of myelin sheaths have a central role. Experimental autoimmune encephalomyelitis (EAE) is a related animal model of multiple sclerosis. Blockade of RAGE by treatment with RAGE isoforms including, but not limited to RAGE isoforms described herein such as SEQ ID NOS: 10-14, can suppress EAE when disease is induced by myelin basic protein (MBP) peptide or encephalitogenic T cells, or when EAE occurs spontaneously in T-cell receptor (TCR)-transgenic mice devoid of endogenous TCR-alpha and TCR-beta chains. Treatment with RAGE isoforms also can decrease infiltration of the CNS by immune and inflammatory cells.

4. Neurodegenerative Disease

RAGE isoforms including, but not limited to, RAGE isoforms described herein such as polypeptides that include the sequence of amino acids set forth in any of SEQ ID NOS: 10-14, can be used in treatment of amyloid diseases, including Alzheimer's disease and related conditions. Alzheimer's disease (AD) is characterized by excessive inflammation and the accumulation of inflammatory proteins in AD brains leading to neurodegeneration and dementia associated with AD. The inflammation is caused by innate immune responses from microglia and astrocytes, resident macrophages of the central nervous system, to aggregated β-amyloid (Aβ) forming senile plaques. The mechanism for Aβ-induced inflammation is due to RAGE signaling as Aβ and other RAGE ligands are accumulated in the AD brain. Treatment with RAGE isoforms can reduce the inflammation associated with AD by acting as antagonists of RAGE/RAGE ligand signaling.

Other neurodegenerative diseases, such as Creutzfeldt-Jakob disease and Huntington's disease, can be treated with RAGE isoforms. RAGE and its ligands are accumulated in prion protein plaques in Creutzfeldt-Jakob disease and in the caudate nucleus in Huntington's disease. Treatment of neurodegenerative diseases with RAGE isoforms can limit inflammation and disease associated with sustained RAGE signaling.

5. Cardiovascular Disease

RAGE isoforms including, but not limited to, RAGE isoforms described herein such as polypeptides that contain sequences of amino acids set forth in any of SEQ ID NOS: 10-14, can be used in treatment of cardiovascular disease. RAGE and its ligands accumulate in ageing tissues including in the ageing human heart leading to sustained and chronic RAGE-mediated signaling. For example, RAGE signaling can mediate regulation of cell-matrix interactions through the activation of matrix metalloproteinases that has been observed, for example, in cardiac fibroblasts associated with cardiac fibrosis. Conversely, decreased levels of a soluble RAGE isoform in the plasma of patients or subjects with coronary artery disease, but not in control subjects, correlates with prognosis of atherosclerosis and vascular inflammation associated with coronary artery disease. Treatment of patients or subjects with cardiovascular disease and related conditions with RAGE isoforms may exert antiatherogenic effects by preventing ligand-mediated RAGE-dependent cellular activation.

6. Kidney Disease

RAGE isoforms including, but not limited to, RAGE isoforms described herein such as polypeptides that contain sequences of amino acids set forth in any of SEQ ID NOS: 10-14, can be used in treatment of chronic kidney disease. Kidney disease is characterized by chronic inflammation and elevated blood levels of proinflammatory cytokines such as TNF-α, IL-1β, and AGE, a ligand for RAGE. RAGE also is accumulated on peripheral blood monocytes from patients and subjects with chronic kidney disease, increasing as renal function deteriorates. RAGE/RAGE ligand signaling is associated with the chronic monocyte-mediated systemic inflammation associated with chronic kidney disease. Treatment with RAGE isoforms can diminish binding of RAGE ligands to cell surface RAGE and attenuate RAGE-mediated signaling such as the production of proinflammatory cytokines like TNF-α.

7. Arthritis

RAGE isoforms including, but not limited to, RAGE isoforms described herein such as polypeptides that contain sequences of amino acids set forth in any of SEQ ID NOS: 10-14, can be used in treatment of arthritis and related conditions. RAGE ligands, such as AGE, are accumulated in the synovial tissues of patients and subjects with rheumatoid and osteoarthritic arthritis. Further, RAGE also is found in the synovial tissue and on a variety of cell types, including macrophages and T cells, of patients and subjects with arthritis. Treatment of subjects, including human patients, with RAGE isoforms can diminish the accelerated inflammation associated with arthritis contributed to by RAGE ligand accumulation and sustained and chronic RAGE signaling.

8. Cancer

RAGE isoforms including, but not limited to, RAGE isoforms described herein, such as polypeptides that contain sequences of amino acids set forth in any of SEQ ID NOS: 10-14, can be used in treatment of cell proliferation diseases including cancers. RAGE signaling contributes to cancer progression by affecting cellular processes such as cell adhesion, cell motility, and the production of matrix proteinases associated with tumor proliferation and invasion. Examples of cancers to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Cancers treatable with RAGE isoforms are generally cancers expressing RAGE receptor. Such cancers can be identified by any means known in the art for detecting RAGE expression, for example by RT-PCR or by immunohistochemistry. Treatment of cancer with RAGE isoforms can suppress tumor growth and metastases. For example, an animal model of tumor cell formation can be produced by injecting C6 glioma cells into immunocompromised athymic nude mice. Administration of RAGE isoforms for example, once daily, to the immunocompromised mice can decrease tumor volume and decrease cellular proliferation at the tumor site. In another model termed the Lewis lung carcinoma model, whereby distant metastases flourish upon removal of the primary tumor, administration of RAGE isoforms just before and just after resection of primary tumors resulting from inoculation with wild-type Lewis lung carcinoma cells results in a decrease in the number of lung surface metastases.

9. Combination Therapies

RAGE isoforms, including those provided herein, such as but not limited to the RAGE isoforms (and encoding nucleic acids) set forth in SEQ ID NOS: 5-9 or 10-14 can be used in combination with each other, with other cell surface receptor isoforms, such as a herstatin or any described, for example, in U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application No WO 00/44403, WO 1/61356, WO 2005/016966, including but not limited, those set forth in SEQ ID Nos.27-92, 104-163, 222-291 and 306-318); and/or with other existing drugs and therapeutics to treat diseases and conditions, particularly those involving aberrant angiogenesis and/or neovascularization, including, but not limited to, cancers and other proliferative disorders, inflammatory diseases, autoimmune disorders, as set forth herein and known to those of skill in the art.

For example, a RAGE isoform can be administered with an agent for treatment of diabetes. Such agents include agents for the treatment of any or all conditions such as diabetic periodontal disease, diabetic vascular disease, tubulointerstitial disease and diabetic neuropathy. In another example, a RAGE isoform is administered with an agent that treats cancers including squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. A RAGE isoform can be administered in combination with an agent that inhibits AGE formation and/or AGE accumulation. For example, a RAGE isoform is administered with a thiazolidine derivative, aminoguanidine or an AGE cross-link breaker such as Alagebrium (3-phenacyl-4,5-dimethylthiazolium chloride, ALT-711). A RAGE isoform can be administered in combination with two or more agents for treatment of a disease or a condition. For example, for treatment of diabetic neuropathy, a RAGE isoform is administered with two or more of a glycation inhibitor, an inhibitor of rennin angiotensis system, antioxidants, a protein kinase C inhibitor and an inhibitor of secretion and action of prosclerotic cytokines such as TGF-β.

Adjuvants and other immune modulators can be used in combination with RAGE isoforms in treating cancers, for example to increase immune response to tumor cells. Combination therapy can increase the effectiveness of treatments and in some cases, create synergistic effects such the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

Combinations of RAGE isoforms with intron fusion proteins and other agents, including cell surface receptor (CSR) polypeptide isoforms for treating cancers and other disorders involving aberrant angiogenesis (see, e.g. FIG. 1 outlining targets in the angiogenesis and neovascularization pathway for such polypeptides and those described herein and in the above-noted copending and published applications U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application No WO 00/44403, WO 1/61356, WO 2005/016966 are provided. The cell surface receptors include receptor tyrosine kinases, such as members of the VEGFR, FGFR, PDGFR (including Rα, Rβ, CSF1R, Kit), Met (including c-Met, c-RON), Tie-2 and EPHA2 families. These also include ERBB2, ERBB3, ERBB4, DDR1, DDR2, EPHA, EPHB, FGFR2, FGFR3, FGFR4, MET, PDGFR, TEK, TIE, KIT, ERBB2, VEGFR1, VEGFR2, VEGFR3, FLT1, FLT3, TNFR1, TNFR2, RON, and CSF1R. Exemplary of such isoforms are the herstatins (see, SEQ ID Nos. 252-265), polypeptides that include the intron portion of a herstatin (see, SEQ ID Nos. 266-291, which set forth the polypeptides and encoding sequences of nucleotides), as well as isoforms set forth in any of SEQ ID Nos. 27-92, 104-163 and 222-251. The combinations of isoforms and/or drug agent and RAGE receptor selected is function of the disease to be treated and is based upon consideration of the target tissues and cells and receptors expressed thereon.

The combinations can target two or more cell surface receptors or steps in the angiogenic and/or endothelial cell maintenance pathways or can target two or more cell surface receptors or steps in a disease process, such as any which one or both of these pathways are implicated, such as inflammatory diseases, tumors and all other noted herein and known to those of skill in the art. The two or more agents can be administered as a single composition or can be administered as two or more compositions (where there are more than two agents) simultaneously, intermittently or sequentially. They be packaged as a kit that contains two or more compositions separately or as a combined composition and optionally with instructions for administration and/or devices for administration, such as syringes.

10. Evaluation of RAGE Isoform Activities

If needed animal models can be used to evaluate RAGE isoforms that are candidate therapeutics. Parameters that can be assessed include, but are not limited to efficacy and concentration-response, safety, pharmacokinetics, interspecies scaling and tissue distribution. Model animal studies include assays such as described herein as well as those known to one of skill in the art. Animal models can be used to obtain date that then can be extrapolated to human dosages for design of clinical trials and treatments with RAGE isoforms, for example, efficacy and concentration-response can be extrapolated from animal model results.

The following examples are included for illustrative purposes only and are not intended to limit the scope of the invention.

### M. EXAMPLES

### Example 1

### Method for Cloning RAGE Isoforms

A. Preparation of Messenger RNA

mRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

mRNA was denatured at 70° C. in the presence of 40% DMSO for 10 min and quenched on ice. First-strand cDNA was synthesized with either 200 ng oligo(dT) or 20 ng random hexamers in a 20-μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of Stratascript reverse transcriptase (Stratagene, La Jolla, Calif.). After incubation at 37° C. for 1 h, the cDNA from both reactions were pooled and treated with 10 units of RNase H (Promega, Madison, Wis.).

C. PCR Amplification

Gene-specific PCR primers were selected using the Oligo 6.6 software (Molecular Biology Insights, Inc., Cascade, Colo.) and synthesized by Qiagen-Operon (Richmond, Calif.). The forward primers flank the start codon. The reverse primers flank the stop codon or were chosen from regions at least 1.5 kb downstream from the start codon (see Table 6). Each PCR reaction contained 10 ng of reverse-transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 35 cycles and 94.5° C. for 45 s, 58° C. for 50 s, and 72° C. for 5 min. The reaction was terminated with an elongation step of 72° C. for 10 min.

TABLE 5
GENEs FOR CLONING RAGE ISOFORMS

| | Catalytic | SEQ ID | | | SEQ ID |
|---|---|---|---|---|---|
| nt ACC. # | Domain | NO: | ORF | pit ACC. # | NO: |
| NM_001136 | | 1 | | NP_001127 | 2 |

TABLE 6

PRIMERS FOR PCR CLONING.

SEQ
ID

| | | |
|---|---|---|
| NO | Primer | Sequence |

| | | |
|---|---|---|
| 15 | RAGE_Fu | CAG GAC CCT GGA AGG AAG CA |
| 16 | RAGE_F1 | AGG ATG GCA GCC GGA ACA G |
| 17 | RAGE_flR1 | CCC CTC AAG GCC CTC CAG TA |
| 18 | RAGE_Intron3R1 | GGA AGT CAG AGG CCC TCA TGG |
| 19 | RAGE_Intron4R1 | GGG AAA GAG TGG TGA CCT CAG A |
| 20 | RAGE_Intron5R1 | CTT GGG GGG CAC CTT AGG ACT C |
| 21 | RAGE_Intron6R1 | ACT CCC TCT TTC CCT AAG GGT CA |
| 22 | RAGE_Intron7R1 | GTT ATG GTT CAC CCT ACC TCC CA |
| 23 | RAGE_Intron8R1 | ATTT AGC TCA GAG GGA AGA AGG GA |

D. Cloning and Sequencing of PCR Products

PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into Escherichia coli. Recombinant plasmids were selected on LB agar plates containing 100 μg/ml carbenicillin. For each transfection, 192 colonies were randomly picked and their cDNA insert sizes were determined by PCR with M13 forward and reverse vector primers. Representative clones from PCR products with distinguishable molecular masses as visualized by fluorescence imaging (Alpha Innotech, San Leandro, Calif.) were then sequenced from both directions with vector primers (M13 forward and reverse). All clones were sequenced entirely using custom primers for directed sequencing completion across gapped regions.

E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding putative RAGE isoforms are studied further (see below, Table 6).

F. Targeted Cloning

Computational analysis of public EST databases identified potential splice variants with intron retention or insertion. Cloning of potential splice variants identified by EST database analysis were to be performed by RT-PCR using primers flanking the putative open reading frame as described above.

G. Exemplary RAGE Isoforms

Exemplary RAGE isoforms, isolated using the methods described herein are shown below in Table 7. Nucleic acid molecules encoding RAGE isoforms are provided and the sequences thereof are set forth in SEQ ID NOS: 5-9. The sequences of polypeptides of RAGE isoforms are set forth in SEQ ID NOS: 10-14.

TABLE 7
RAGE Isoforms

| ID | Isoform Type | Length | SEQ ID NO |
|---|---|---|---|
| SR021A05 | RAGE | 146 | 10 |
| SR021C02 | RAGE | 266 | 13 |
| SR021C06 | RAGE | 387 | 12 |
| SR021C08 | RAGE | 173 | 14 |
| SR021F06 | RAGE | 172 | 11 |

### Example 2

### RAGE Isoform Expression and Activity Assays

A. Analysis of mRNA Expression

Expression of the cloned RAGE isoforms is determined by RT-PCR (or quantitative PCR) in various tissues using the variant-specific primers (such as set forth in Example 1, Table 5).

Expression of the cloned RAGE isoforms is determined by RT-PCR (or quantitative PCR) in various tissues including: brain, heart, kidney, placenta, prostate, spleen, spinal cord, trachea, testis, uterus, fetal brain, fetal liver, adrenal gland, liver, lung, small intestine, salivary gland, skeletal muscle, thymus, thyroid and a variety of tumor tissues including: breast, colon, kidney, lung, ovary, stomach, uterus, MDA435 and HEPG2. PCR primers (such as set forth in Example 1, Table 5) are selected within the exclusive regions of retained introns or alternative exons, such that only the soluble receptor-specific signals are amplified. Each PCR reaction is performed with 2 cycle numbers (e.g. 32 versus 38 cycles) for the purpose of getting semi-quantitative results. Expression of each cloned CSR isoform is compared to the expression of the corresponding wildtype membrane receptor.

B. Expression

Sequence-verified RAGE-IFP encoding cDNA molecules were each subcloned into a replication-deficient recombinant adenoviral vector under control of the CMV promoter, following the manufacturer's instruction (Invitrogen, Cat# K4930-00). The recombinant adenoviruses were produced using 293A cells (Invitrogen). Supernatants from the infected 293 cells were analysed by immunoblotting using an anti-Myc antibody. The results show that the RAGE-IFPs were efficiently expressed and secreted in 293 cells.

C. Secretion

RAGE isoforms are analyzed in cultured human cells to assess for secreted isoforms. Splice variant cDNAs encoding candidate RAGE isoforms are subcloned into a mammalian expression vector, such as the pcDNA3 vector (Invitrogen, Carlsbad, Calif.) with a myc tag fused at the C-terminus of the proteins to facilitate their detection.

Human embryonic kidney 293T cells are seeded at 2×10⁶ cells/well in a 6-well plate and maintained in Dulbecco's modified Eagle's medium and 10% fetal bovine serum (Invitrogen). Cells are transfected using LipofectAMINE 2000 (Invitrogen) following the manufacturer's instructions. On the day of transfection, 5 μg plasmid DNA is mixed with 15 μl of LipofectAMINE 2000 in 0.5 ml of the serum-free DMEM. The mixture is incubated for 20 minutes at room temperature before it is added to the cells. Cells are incubated at 37° C. in a CO₂ incubator for 48 hours. To study the transgene expression of the secreted RAGE isoforms, the supernatants are collected and the cells lysed in PBS buffer containing 0.2% of Triton X-100. Both the cell lysates and the supernatants are assayed for the transgene expression. Purified His6-tagged proteins are eluted and separated on SDS-polyacrylamide gels for immunoblotting using anti-Myc antibodies (both from Invitrogen). Antibodies are diluted 1:5000. Expression of the secreted RAGE isoforms is detected in cell lysates and conditioned media by Western blot using an anti-Myc antibody.

D. Receptor Binding

Co-immunoprecipitation assays were performed to show binding of RAGE isoforms and secreted RAGE isoforms to their respective membrane anchored full-length receptors (see, for example, Jin et al. J Biol Chem 2004, 279:1408 and Jin et al. J Biol Chem 2004, 279:14179). Human embryo kidney 293T cells are transiently transfected with the recombinant pcDNA 3.1 (MycHis) plasmid expressing soluble RAGE (as described above). Forty-eight hours after transfection, conditioned medium is collected and binding of RAGE ligand is assessed. Conditioned medium (100 μl) from transfected 293T cells is incubated with RAGE ligand (100 ng) in the presence or absence of 2 μg of soluble RAGE-Fc (R&D Systems) for one hour. Protein complexes are immunoprecipitated with 0.2 μg/reaction of anti-RAGE ligand antibodies (R&D Systems) and separated on a denaturing protein gel probed with anti-Myc antibody. The Western blot shows protein binding between sRAGE-Myc and RAGE ligand.

E. Proliferation Assays

Modulation of cell proliferation by RAGE isoforms can be assessed in cells transformed with a RAGE isoform. Cells, seeded at a predetermined density, such as ECV304 cells, are transformed with a RAGE cDNA or control (such as a wildtype/predominant form of RAGE and/or vector alone). After an incubation and attachment period, ligand is added and the cells are incubated again. Cell proliferation can then be assessed, for example using a 3-(4,5-dimethylthiazol-2-yl)-2,5 diphenyl-2H-tetrazolium bromide (MTT) method. (see Yonekura et al. 2003 Biochem J. 370:1097-1109).

F. Complexation

RAGE isoforms can be assayed for their ability to complex with other proteins. In one example, a RAGE isoform can be assessed for complexation with LF-L (lactoferrin-like AGE binding protein) using a ligand blotting assay (see e.g., Schmidt et al. 1994 J. Biol. Chem. 269: 9882-88). LF-L radiolabeled with ¹²⁵I (¹²⁵I-LF-L) is incubated with RAGE protein (isoform and/or wildtype form) immobilized on a solid support After washing, the amount of ¹²⁵I-LF-L associated with a RAGE isoform can be quantified.

Complexation also can be assessed in a competitive assay. RAGE is adsorbed onto polypropylene tubes such that it remains tightly bound to the tubes (see Schmidt et al. 1994 J. Biol. Chem. 269: 9882-88). ¹²⁵I-LF-L is added to the tubes alone or after preincubation with a RAGE isoform. After an incubation period, the tubes are washed and the amount of ¹²⁵I-LF-L binding is assessed by measuring the radioactivity associated with each tube. A comparison of the samples that were preincubated with a RAGE isoform versus no preincubation indicates whether the RAGE isoform competes effectively for binding to LF-L.

G. ERK Phosphorylation Assays

RAGE isoforms can be assessed for their ability to stimulate ERK phosphorylation. Endothelial cells (human microvascular EC cells) expressing RAGE or a RAGE isoform are incubated in serum-free media and then AGEs are added for an incubation period. After washing, cells are solubilized and extracts subject to SDS-PAGE. Proteins are transferred to a membrane and the amount of phosphorylated ERK is assessed by immunoreactivity with an ant-phosphoERK antibody.

H. Cell Migration Assay

RAGE isoform effects on cell migration can be assessed. Cells, such as ECV304 cells, are stably transformed with a RAGE isoform cDNA or control (e.g. a wildtype/predominant form of RAGE and/or vector alone). The stably transformed cells are seeded onto plates and grown to confluence. Cells are wounded by denuding a strip of the monolayer of cells. After washing in serum free media, the cells are incubated with media containing serum and type I collagen. Cell cultures are photographed over time to monitor the rate of wound closure (i.e. cell migration into the wounded strip area).

I. Neurite Outgrowth

RAGE-mediated affects on neurite outgrowth can be assessed for a RAGE isoform by stably transforming a neuroblastoma cell line with a RAGE cDNA or control. The cells are serum starved and grown overnight on amphoterin coated glass slides. Filamentous actin is stained, for example using TRITC-phalloidin and the percentage of cells bearing neuritis is assessed and compared between samples. Cells also can be stained with an antibody against RAGE (or against a tag if tagged-RAGE is expressed, e.g. a myc tag) to assess the proportion of cells expressing a RAGE isoform that formed neurite outgrowths (see for example, Huttunen et al. 1999 J. Biol. Chem. 274:19919-24).

### Example 3

### Preparation and Expression of RAGE Intron Fusion Protein Construct in Human Cells

A. Generation of tPA cDNA

In order to obtain human tissue plasminogen activator (tPA) cDNA, PCR primers specific for the 5′ portion of the human tissue plasminogen activator (tPA) including the tPA signal/pro sequence (based on the human tPA cDNA sequence as set forth in SEQ ID NO: 327) were selected based on the published information (Kohne et al. (1999) J Cellular Biochem 75:446-461) and synthesized by Qiagen-Operon (Richmond, Calif.). The sequences of the primers are set forth in SEQ ID NO: 349 and SEQ ID NO: 350 (see Table 9.) Each PCR reaction contained 10 ng of reverse transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 35 cycles at 94.5° C. for 45 s, 58° C. for 50 s, and 72° C. for 5 min. The reaction was terminated with an elongation step of 72° C. for 10 min. PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (SEQ ID NO:351, Qiagen), and transformed into Escherichia coli for purification of the pDrive-tPA vector.

B. PCR Amplification and Expression Cloning of the tPA Signal/Pro Sequence

In order to clone the portion of the nucleic acid that includes the nucleotides encoding the tPA signal/pro sequence (see Table 8) as set forth in SEQ ID NO: 328, PCR was performed using the primers as forth in SEQ ID NO. 352 and SEQ ID NO. 353 (see Table 9). The primers were generated to contain restriction enzyme cleavage sites for NheI and XhoI, as well as a myc-tag, to facilitate cloning of the amplified product into the pCI expression plasmid (SEQ ID NO: 354, Promega). The PCR reaction was performed as above with 10 ng pDrive-tPA. The PCR conditions included 35 cycles at 94.5° C. for 45 s, 58° C. for 50 s, and 72° C. for 5 min. The reaction was terminated with an elongation step of 72° C. for 10 min. The tPA encoded cDNA was digested with NheI and XhoI to generate the tPA signal/pro sequence fragment and subcloned into the pCI expression plasmid (Promega) at the NheI and XhoI sites to form the pCI-tPA:myc vector.

TABLE 8
LIST OF GENES FOR CLONING tPA-intron
fusion protein CONSTRUCTs

| | | SEQ ID | | | SEQ ID |
|---|---|---|---|---|---|
| nt ACC. # | Description | NO: | ORF | prt ACC. # | NO: |
| NM_000930 | tPA | 327 | | NP_000921 | 326 |
| | tPA pre/pro | 329 | | | 328 |
| | sequence | | | | |

C. Cloning of Intron Fusion Proteins into the pCI-tPA Vector

Intron fusion proteins were PCR amplified from their pDrive sequencing vector, respectively, and subsequently cloned into the pCI-tPA:myc vector. For the PCR amplification, the forward primers contain an XhoI site, and the reverse primers contain a NotI site. The RAGE intron fusion protein without a signal sequence (see e.g., 13) was PCR amplified using primers set forth in SEQ ID NOS:355 and 356. Each PCR reaction contained 10 ng of reverse transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 25 cycles and 94.5° C. for 45 s, 58° C. for 50 s, and 72° C. for 5 min. The reaction was terminated with an elongation step of 72° C. for 10 min. PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), subcloned into the pCI-tPA:myc vector at the XhoI and NotI sites downstream of the tPA/pro sequence to generate tPA:myc-intron fusion protein constructs as set forth in SEQ ID NOs. 340 (nucleotide) and 341 (amino acid). An exemplary tPA-intron fusion protein of a RAGE isoform is set forth in Table 10.

TABLE 9

PRIMERS FOR PCR CLONING.

SEQ
ID

| | | | |
|---|---|---|---|
| NO | Primer | ID | Sequence |

| | | | |
|---|---|---|---|
| 349 | tPA_F | | CTCTGCGAGGAAAGGGAAGGA |
| 350 | tPA_R | | CGTGCCCCTGTAGCTGATGCC |
| 352 | tPApre/pro_F1 | | ATTAGCTAGCCACCATGGATGCAA |
| | | | TGAAGAGAGGGATTACTCGAGCAG |
| | | | ATCCTCTTGTGAGATGAGTTTTTG |
| | | | TTCTG |
| 353 | tPApre/pro_R1 | | GCTCCTCTTCGAATCG |
| 355 | RAGEIFP_F | SR021_C02 | AATTCTCGAGCAAAACATCACAGC |
| | | | CCGGA |
| 356 | RAGEIFP_R | SR021_C02 | AATTGAATTCCTAAGGGTCAGACT |
| | | | TCCAGA |

TABLE 10
tPA-intron fusion protein Fusions

| | | SEQ ID NO | SEQ ID NO |
|---|---|---|---|
| ID | Isoform Type | (nucleotide) | (amino acid) |
| SR021C02 | tPA-myc-RAGE | 340 | 341 |

D. Protein Expression and Secretion

Medium from cultured human cells was assessed for secretion of each of the tPA-intron fusion proteins. To express the tPA-intron fusion proteins in human cells, human embryonic kidney 293T cells were seeded at 2×10⁶ cells/well in a 6-well plate and maintained in Dulbecco's modified Eagle's medium (DMEM) and 10% fetal bovine serum (Invitrogen). Cells were transfected using LipofectAMINE 2000 (Invitrogen) following the manufacturer's instructions. On the day of transfection, 5 μg plasmid DNA was mixed with 15 μl of LipofectAMINE 2000 in 0.5 ml of serum-free DMEM. The mixture was incubated for 20 minutes at room temperature before it was added to the cells. Cells were incubated at 37° C. in a CO₂ incubator for 48 hours. To study the protein secretion of intron fusion proteins, the conditioned media was collected 48 hours after transfection and expression levels were analyzed by Western blotting. Conditioned media was analyzed by separation on SDS-polyacrylamide gels followed by immunoblotting using an anti-Myc antibody (Invitrogen). Antibodies were diluted 1:5000. To study the cellular protein expression of the intron fusion proteins, after cell culture media was removed, the transfected cells were harvested and lysed in a cell lysis buffer (PBS/0.25% Triton X-100). Lysates were clarified by centrifugation to remove insoluble cell debris. Typically, 10 micrograms of proteins from each samples were separated on SDS-PAGE gels after protein concentrations were determined. Cell lysates were analyzed by Western blotting using an anti-Myc antibody (Invitrogen). Expression and secretion of intron fusion proteins containing a tpA pre/prosequence were compared to intron fusion proteins containing the original or endogenous signal peptide. Comparisons of expression and secretion of an exemplary RAGE isoform intron fusion protein is depicted in Table 11.

TABLE 11
Summary of intron fusion protein
Protein Expression and Secretion

| intron | | Protein | Protein | | |
|---|---|---|---|---|---|
| fusion | | Expression | Secretion | Protein | Protein |
| protein | | w/Original | w/Original | Expression | Secretion |
| ID | Gene | sp | sp | w/tPA sp | w/tPA sp |
| SR021C02 | RAGE | ++ | + | +++ | +++ |

Since modifications will be apparent to those of skill in this art, it is intended that this invention be limited only by the scope of the appended claims

1. An isolated Receptor for Advanced Glycation Endproducts (RAGE) isoform polypeptide, wherein:
the RAGE isoform is an intron fusion protein, wherein the intron portion is encoded by a sequence of nucleotides that includes all or a portion of an intron selected from among introns 2, 3, 5 and 8 of a cognate RAGE gene.

2. The isolated RAGE isoform polypeptide of claim 1, wherein the sequence of the cognate RAGE gene is set forth in SEQ ID NO:325, or is an allelic or species variant thereof.

3. The RAGE isoform polypeptide of claim 1, wherein the isoform comprises a sequence of amino acids set forth in any one of SEQ ID NOS: 10-12, or is an allelic or species variant thereof.

4. An isolated Receptor for Advanced Glycation Endproducts (RAGE) isoform polypeptide, comprising:
a deletion and/or insertion of one or more amino acids of the first C-type Ig-like domain of RAGE;
a deletion and/or insertion of one or more amino acids of the second C-type Ig-like domain of RAGE;
a deletion and/or insertion of one or more amino acids of the transmembrane domain of RAGE, wherein:
the membrane localization of the RAGE isoform is reduced or abolished compared to RAGE; and
the RAGE isoform is an intron fusion protein.

5. The RAGE isoform polypeptide of claim 4, wherein the isoform has a sequence of amino acids set forth in any one of SEQ ID NOS: 10, 11, 13, or 14, or is an allelic or species variant thereof.

6. An isolated Receptor for Advanced Glycation Endproducts (RAGE) isoform polypeptide that comprises a sequence of amino acids selected from among:
a) a sequence that comprises at least 70% of the amino acid sequence set forth in SEQ ID NO: 10 and that has at least 70% sequence identity with a sequence of amino acids set forth in SEQ ID NO: 10;
b) a sequence that comprises at least 75% of the amino acid sequence set forth in SEQ ID NO: 11 and that has at least 75% sequence identity with a sequence of amino acids set forth in SEQ ID NO: 11;
c) a sequence that comprises at least 86% of the amino acid sequence set forth in SEQ ID NO: 12 and that has at least 86% sequence identity with a sequence of amino acids set forth in SEQ ID NO: 12;
d) a sequence that comprises at least 90% of the amino acid sequence set forth in SEQ ID NO:13 and that has at least 90% sequence identity with a sequence of amino acids set forth in SEQ ID NO: 13; and
e) a sequence that comprises at least 93% of the amino acid sequence set forth in SEQ ID NO: 14 and that has at least 93% sequence identity with a sequence of amino acids set forth in SEQ ID NO: 14, wherein:
sequence identity is compared along the full length of each SEQ ID to the full length sequence of the RAGE isoform.

7. The RAGE isoform polypeptide of claim 6, wherein sequence identity is compared with a mature isoform that lacks a signal sequence.

8. The RAGE isoform polypeptide of claim 6, wherein sequence identity is compared with a precursor form that includes a signal sequence.

9. The RAGE isoform polypeptide of claim 6, that is encoded by a nucleic acid molecule that comprises at least one codon from an intron, wherein the intron is from a gene encoding RAGE.

10. A RAGE isoform polypeptide of any of claim 1, claim 4 or claim 6, but lacking the signal peptide.

11. The RAGE isoform polypeptide of any of claims 1, claim 4 or claim 6, wherein the isoform comprises a signal peptide.

12. The RAGE isoform polypeptide of claim 3 or claim 5, wherein the allelic variant comprises variations that correspond to one or more of the allelic variations denoted in SEQ ID NO: 4.

13. The RAGE isoform polypeptide of claim 1, claim 4 or claim 6, wherein the RAGE isoform contains the same number of amino acids as any of SEQ ID NOS: 10-14, or the same number but lacking the signal sequence in each.

14. A RAGE isoform polypeptide of claim 1, claim 4, or claim 6 that is encoded by a sequence of nucleotides set forth in SEQ ID NOS: 5-9 or an allelic or species variant thereof.

15. The RAGE isoform polypeptide of claim 14, wherein the allelic variant comprises variations that correspond to one or more nucleotides of the allelic variations denoted in SEQ ID NO: 3.

16. The RAGE isoform polypeptide of any of claims 1, 4 and 6, wherein the isoform modulates a function or activity of a RAGE receptor.

17. The RAGE isoform polypeptide of claim 16, wherein the activity of a RAGE modulated by the polypeptide is selected from among one or more of: ligand binding, competition with RAGE for ligand binding, ligand endocytosis, regulation of gene expression, signal transduction, interaction with a signal transduction molecule, membrane association and membrane localization.

18. A RAGE isoform polypeptide that has at least 70%, 75%, 80%, 85%, 90%, 95%, 96%, 97%, 98%, 99% or more sequence identity to a polypeptide of claim 1 or claim 4.

19. A pharmaceutical composition, comprising a RAGE isoform of any of claims 1, 4, and 6.

20. The composition of claim 19, comprising an amount of the isoform effective for modulating an activity of a cell surface receptor.

21. The composition of claim 19, wherein the isoform modulates a function or activity of a RAGE.

22. The composition of claim 19, wherein the activity modulated by the polypeptide is one or more of: ligand binding, competition with RAGE for ligand binding, ligand endocytosis, regulation of gene expression, signal transduction, interaction with a signal transduction molecule, membrane association and membrane localization.

23. The composition of claim 20, wherein modulation is an inhibition of activity.

24. The composition of claim 19, wherein the isoform of the composition complexes with a RAGE.

25. A nucleic acid molecule encoding a RAGE isoform of any of claims 1, 4, and 6.

26. A nucleic acid molecule of claim 25, comprising an intron and an exon, wherein:
the intron contains a stop codon;
the nucleic acid molecule encodes an open reading frame that spans an exon intron junction; and
the open reading frame terminates at the stop codon in the intron.

27. The nucleic acid molecule of claim 26, wherein the intron encodes one or more amino acids of the encoded RAGE isoform.

28. The nucleic acid molecule of claim 26, wherein the stop codon is the first codon in the intron.

29. An isolated nucleic acid molecule of claim 25, comprising a sequence of nucleotides set forth in any one of SEQ ID NOS: 5-9 or an allelic or species variant thereof.

30. A vector, comprising the nucleic acid molecule of claim 25.

31. The vector of claim 30 that is a mammalian vector.

32. The vector of claim 31 that is a viral vector.

33. The vector of claim 30 that is episomal or that integrates into the chromosome of a cell into which it is introduced.

34. A cell, comprising the vector of claim 30

35. A pharmaceutical composition, comprising a vector of claim 30.

36. A method of treating a disease or condition comprising, administering a pharmaceutical composition of claim 19 to a subject.

37. The method of claim 36, wherein the disease or condition is selected from among diabetes, diabetes-related conditions, cancers, inflammatory diseases, angiogenesis-related conditions, cell proliferation-related conditions, immune disorders, kidney disease, ocular disease, endometriosis, periodontal disease and neurodegenerative diseases.

38. The method of claim 37, wherein the disease or condition is selected from among rheumatoid arthritis, osteoarthritic arthritis, multiple sclerosis, Alzheimer's disease, Creutzfeldt-Jakob disease, Huntington's disease, and posterior intraocular inflammation, uveitic disorders, ocular surface inflammatory disorders, macular degeneration, neovascular disease, proliferative vitreoretinopathy, atherosclerosis, type I diabetes, multiple sclerosis and chronic kidney disease.

39. The method of claim 37, wherein the diabetes-associated condition is selected from periodontal disease, autoimmune disease, vascular disease, tubulointerstitial disease, atherosclerosis and vascular disease associated with wound healing.

40. The method of claim 37, wherein the cancer is selected from the group consisting of carcinoma, lymphoma, blastoma, sarcoma, leukemia, lymphoid malignancies, squamous cell cancer, lung cancer including small-cell lung cancer, non-small cell lung cancer, adenocarcinoma of the lung and squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial/uterine carcinoma, salivary gland carcinoma, renal cancer, prostate cancer, vulval cancer, thyroid cancer, hepatic carcinoma, anal carcinoma, penile carcinoma, and head and neck cancer.

41. The method of claim 36, wherein the pharmaceutical composition contains a polypeptide that inhibits angiogenesis, cell proliferation, cell migration, tumor cell growth or tumor cell metastasis.

42. The method of claim 36, wherein the disease is an angiogenesis-related disease.

43. The method of claim 36, wherein the disease is selected from among inflammatory and immune disorders.

44. The method of claim 43, wherein the disease is selected from among
diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease, Alzheimers disease and other neurodegenerative diseases.

45. A method of inhibiting tumor invasion or metastasis of a tumor, comprising administering a composition of claim 19 to a subject.

46. A conjugate, comprising a RAGE isoform or an active fragment thereof.

47. The conjugate of claim 46, comprising a RAGE isoform or fragment thereof linked to a multimerization domain.

48. The conjugate of claim 47, wherein the multimerization domain is selected from among an Fc region, a leucine zipper, an amino acid sequence comprising a protuberance complementary to an amino acid sequence comprising a hole, a hydrophobic domain, a hydrophilic domain, an amino acid sequence comprising a free thiol moiety which reacts to form an intermolecular disulfide bond with a multimerization domain of an additional amino acid sequence, and a protein interaction domain selected from among an R subunit of a PKA and an anchoring domain (AD).

49. A conjugate, comprising a RAGE isoform of any of claims 1, 4, and 6.

50. A multimeric compound, comprising one or more conjugates of claim 47 and one or more other cell surface receptor isoforms or active fragments thereof linked to a multimerization domain, whereby the resulting compound modulates the activity of a RAGE and/or a CSR.

51. The multimeric compound of claim 50 that is a homodimer or a heterodimer or a trimer.

52. The multimeric compound of claim 50, comprising a RAGE isoform or a domain thereof or a ligand binding portion thereof and a cell surface receptor isoform or a domain thereof or a ligand binding portion or ligand isoform.

53. A chimeric polypeptide, comprising all or at least one domain of a RAGE isoform or an active fragment thereof and all of or at least one domain of a different RAGE isoform or of another cell surface receptor isoform or of a ligand isoform or portion of an isoform that possesses an activity.

54. The chimeric polypeptide of claim 53, wherein the cell surface receptor isoform is an intron fusion protein.

55. The chimeric polypeptide of claim 54, that comprises all of or at least one domain of a RAGE isoform and an intron-encoded portion of a cell surface receptor isoform.

56. A pharmaceutical composition, comprising a polypeptide or conjugate or multimeric compound of any of claims 46, 50 and 53.

57. A method of treating a disease or condition comprising, administering a pharmaceutical composition of claim 56, wherein the disease or condition is mediated by or involves a CSR in its etiology.

58. A combination comprising:
one or more RAGE isoform(s) and one or more other cell surface receptor (CSR) isoforms and/or a therapeutic drug.

59. The combination of claim 58, wherein the isoforms and/or drugs are in separate compositions or in a single composition.

60. A method of treatment, comprising administering the components of the combination of claim 58, wherein each component is administered separately, simultaneously, intermittently, in a single composition or combinations thereof.

61. The method of claim 36, wherein:
the composition comprises a nucleic acid molecule or a vector; and the method comprises:
introducing the composition into a cell(s) that have been removed from a host animal; and
introducing the cells into the same animal or into an animal compatible with the animal from whom the cells were removed or an animal that has been treated to be compatible.

62. The method of any of claims 61, wherein the animal is a human.

63. The conjugate of claim 46, wherein:
the conjugate comprises a RAGE isoform or domain thereof or functional portion thereof, and a second portion from a different RAGE isoform or from another cell surface receptor (CSR);
one of the portions is all or part of an extracellular domain of an isoform; and
the portions are linked directly or via a linker.

64. The conjugate of claim 63, wherein the CSR is a receptor tyrosine kinase.

65. The conjugate of claim 63, wherein one portion is from a herstatin polypeptide.

66. A polypeptide, comprising a domain of RAGE or a RAGE isoform or active fragment thereof linked directly or indirectly to serum albumin or other mucin.

67. The polypeptide of claim 66, wherein the RAGE isoform is an intron fusion protein.

68. The combination of claim 58, chimera of claim 53, or multimer of claim 50, wherein the CSR isoform is an isoform of a ErbB, a VEGFR, a FGFR, a TNFR, a PDGFR, a MET, a Tie-2 or an EPHA2.

69. The combination, conjugate, chimera, or multimeric compound of any of claims 46, 50, 53, or 58 wherein the RAGE or RAGE isoform and/or the CSR isoform or other isoform is an extracellular domain or a portion thereof that possess ligand binding activity or dimerization activity or other activity of a RAGE or CSR.

70. The combination, chimera, or multimer of claim 68, wherein the RAGE or RAGE isoform and/or the CSR isoform or other isoform is an extracellular domain or a portion thereof that possesses ligand binding activity or dimerization activity or other activity of a RAGE or CSR.

71. A pharmaceutical composition, comprising a nucleic acid molecule of claim 25.

72. A cell, comprising a nucleic acid molecule of claim 29.

73. A pharmaceutical composition, comprising a cell of claim 72.

74. A method of treating a disease or condition comprising, administering a pharmaceutical composition of claim 35 to a subject.

75. A method of treating a disease or condition comprising, administering a pharmaceutical composition of claim 71 to a subject.

76. A method of treating a disease or condition comprising, administering a pharmaceutical composition of claim 73 to a subject.

77. A method of treating a disease or condition comprising, administering a cell of claim 72 to a subject.

78. A method of inhibiting tumor invasion or metastasis of a tumor, comprising administering a cell of claim 72 to a subject.

79. A method of inhibiting tumor invasion or metastasis of a tumor, comprising administering a composition of claim 35 to a subject.

80. A method of inhibiting tumor invasion or metastasis of a tumor, comprising administering a composition of claim 71 to a subject.
